SEQUENCE LISTING (9 sequences)

SEQ ID NO: 1 - protein, 2005 aa, Homo sapiens
MAQSVLVPPGPDSFRFFTRESLAAIEQRIAEEKAKRPKQERKDEDDENGPKPNSDLEAGKSLPFIYGDIPPEMVSVPLEDLDPYYINKKTFIVLNKGKAISRFSATPALYILTPFNPIRKLAIKILVHSLFNMLIMCTILTNCVFMTMSNPPDWTKNVEYTFTGIYTFESLIKILARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCLQWPPDNSSFEINITSFFNNSLDGNGTTFNRTVSIFNWDEYIEDKSHFYFLEGQNDALLCGNSSDAGQCPEGYICVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMLEQLKKQQEEAQAAAAAASAESRDFSGAGGIGVFSESSSVASKLSSKSEKELKNRRKKKKQKEQSGEEEKNDRVRKSESEDSIRRKGFRFSLEGSRLTYEKRFSSPHQSLLSIRGSLFSPRRNSRASLFSFRGRAKDIGSENDFADDEHSTFEDNDSRRDSLFVPHRHGERRHSNVSQASRASRVLPILPMNGKMHSAVDCNGVVSLVGGPSTLTSAGQLLPEGTTTETEIRKRRSSSYHVSMDLLEDPTSRQRAMSIASILTNTMEELEESRQKCPPCWYKFANMCLIWDCCKPWLKVKHLVNLVVMDPFVDLAITICIVLNTLFMAMEHYPMTEQFSSVLSVGNLVFTGIFTAEMFLKIIAMDPYYYFQEGWNIFDGFIVSLSLMELGLANVEGLSVLRSFRLLRVFKLAKSWPTLNMLIKIIGNSVGALGNLTLVLAIIVFIFAVVGMQLFGKSYKECVCKISNDCELPRWHMHDFFHSFLIVFRVLCGEWIETMWDCMEVAGQTMCLTVFMMVMVIGNLVVLNLFLALLLSSFSSDNLAATDDDNEMNNLQIAVGRMQKGIDFVKRKIREFIQKAFVRKQKALDEIKPLEDLNNKKDSCISNHTTIEIGKDLNYLKDGNGTTSGIGSSVEKYVVDESDYMSFINNPSLTVTVPIAVGESDFENLNTEEFSSESDMEESKEKLNATSSSEGSTVDIGAPAEGEQPEVEPEESLEPEACFTEDCVRKFKCCQISIEEGKGKLWWNLRKTCYKIVEHNWFETFIVFMILLSSGALAFEDIYIEQRKTIKTMLEYADKVFTYIFILEMLLKWVAYGFQVYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINYTTGEMFDVSVVNNYSECKALIESNQTARWKNVKVNFDNVGLGYLSLLQVATFKGWMDIMYAAVDSRNVELQPKYEDNLYMYLYFVIFIIFGSFFTLNLFIGVIIDNFNQQKKKFGGQDIFMTEEQKKYYNAMKKLGSKKPQKPIPRPANKFQGMVFDFVTKQVFDISIMILICLNMVTMMVETDDQSQEMTNILYWINLVFIVLFTGECVLKLISLRYYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSGPPDCDPDKDHPGSSVKGDCGNPSVGIFFFVSYIIISFLVVVNMYIAVILENFSVATEESAEPLSEDDFEMFYEVWEKFDPDATQFIEFAKLSDFADALDPPLLIAKPNKVQLIAMDLPMVSGDRIHCLDILFAFTKRVLGESGEMDALRIQMEERFMASNPSKVSYEPITTTLKRKQEEVSAIIIQRAYRRYLLKQKVKKVSSIYKKDKGKECDGTPIKEDTLIDKLNENSTPEKTDMTPSTTSPPSYDSVTKPEKEKFEKDKSEKEDKGKDIRESKK

SEQ ID NO: 2 - DNA, 8630 bp, Homo sapiens
  source 1..8630: mol_type = genomic DNA
aagcatgatggaattttagctgcagtcttcttggtgccagcttatcaatcccaaactctgggtgtaaaagattctacagggcactttcttatgcaaggagctaaacagtgattaaaggagcaggatgaaaagatggcacagtcagtgctggtaccgccaggacctgacagcttccgcttctttaccagggaatcccttgctgctattgaacaacgcattgcagaagagaaagctaagagacccaaacaggaacgcaaggatgaggatgatgaaaatggcccaaagccaaacagtgacttggaagcaggaaaatctcttccatttatttatggagacattcctccagagatggtgtcagtgcccctggaggatctggacccctactatatcaataagaaaacgtttatagtattgaataaagggaaagcaatctctcgattcagtgccacccctgccctttacattttaactcccttcaaccctattagaaaattagctattaagattttggtacattctttattcaatatgctcattatgtgcacgattcttaccaactgtgtatttatgaccatgagtaaccctccagactggacaaagaatgtggagtatacctttacaggaatttatacttttgaatcacttattaaaatacttgcaaggggcttttgtttagaagatttcacatttttacgggatccatggaattggttggatttcacagtcattacttttgcatatgtgacagagtttgtggacctgggcaatgtctcagcgttgagaacattcagagttctccgagcattgaaaacaatttcagtcattccaggcctgaagaccattgtgggggccctgatccagtcagtgaagaagctttctgatgtcatgatcttgactgtgttctgtctaagcgtgtttgcgctaataggattgcagttgttcatgggcaacctacgaaataaatgtttgcaatggcctccagataattcttcctttgaaataaatatcacttccttctttaacaattcattggatgggaatggtactactttcaataggacagtgagcatatttaactgggatgaatatattgaggataaaagtcacttttattttttagaggggcaaaatgatgctctgctttgtggcaacagctcagatgcaggccagtgtcctgaaggatacatctgtgtgaaggctggtagaaaccccaactatggctacacgagctttgacacctttagttgggcctttttgtccttatttcgtctcatgactcaagacttctgggaaaacctttatcaactgacactacgtgctgctgggaaaacgtacatgatattttttgtgctggtcattttcttgggctcattctatctaataaatttgatcttggctgtggtggccatggcctatgaggaacagaatcaggccacattggaagaggctgaacagaaggaagctgaatttcagcagatgctcgaacagttgaaaaagcaacaagaagaagctcaggcggcagctgcagccgcatctgctgaatcaagagacttcagtggtgctggtgggataggagttttttcagagagttcttcagtagcatctaagttgagctccaaaagtgaaaaagagctgaaaaacagaagaaagaaaaagaaacagaaagaacagtctggagaagaagagaaaaatgacagagtccgaaaatcggaatctgaagacagcataagaagaaaaggtttccgtttttccttggaaggaagtaggctgacatatgaaaagagattttcttctccacaccagtccttactgagcatccgtggctcccttttctctccaagacgcaacagtagggcgagccttttcagcttcagaggtcgagcaaaggacattggctctgagaatgactttgctgatgatgagcacagcacctttgaggacaatgacagccgaagagactctctgttcgtgccgcacagacatggagaacggcgccacagcaatgtcagccaggccagccgtgcctccagggtgctccccatcctgcccatgaatgggaagatgcatagcgctgtggactgcaatggtgtggtctccctggtcgggggcccttctaccctcacatctgctgggcagctcctaccagagggcacaactactgaaacagaaataagaaagagacggtccagttcttatcatgtttccatggatttattggaagatcctacatcaaggcaaagagcaatgagtatagccagtattttgaccaacaccatggaagaacttgaagaatccagacagaaatgcccaccatgctggtataaatttgctaatatgtgtttgatttgggactgttgtaaaccatggttaaaggtgaaacaccttgtcaacctggttgtaatggacccatttgttgacctggccatcaccatctgcattgtcttaaatacactcttcatggctatggagcactatcccatgacggagcagttcagcagtgtactgtctgttggaaacctggtcttcacagggatcttcacagcagaaatgtttctcaagataattgccatggatccatattattactttcaagaaggctggaatatttttgatggttttattgtgagccttagtttaatggaacttggtttggcaaatgtggaaggattgtcagttctccgatcattccggctgctccgagttttcaagttggcaaaatcttggccaactctaaatatgctaattaagatcattggcaattctgtgggggctctaggaaacctcaccttggtattggccatcatcgtcttcatttttgctgtggtcggcatgcagctctttggtaagagctacaaagaatgtgtctgcaagatttccaatgattgtgaactcccacgctggcacatgcatgactttttccactccttcctgatcgtgttccgcgtgctgtgtggagagtggatagagaccatgtgggactgtatggaggtcgctggccaaaccatgtgccttactgtcttcatgatggtcatggtgattggaaatctagtggttctgaacctcttcttggccttgcttttgagttccttcagttctgacaatcttgctgccactgatgatgataacgaaatgaataatctccagattgctgtgggaaggatgcagaaaggaatcgattttgttaaaagaaaaatacgtgaatttattcagaaagcctttgttaggaagcagaaagctttagatgaaattaaaccgcttgaagatctaaataataaaaaagacagctgtatttccaaccataccaccatagaaataggcaaagacctcaattatctcaaagacggaaatggaactactagtggcataggcagcagtgtagaaaaatatgtcgtggatgaaagtgattacatgtcatttataaacaaccctagcctcactgtgacagtaccaattgctgttggagaatctgactttgaaaatttaaatactgaagaattcagcagcgagtcagatatggaggaaagcaaagagaagctaaatgcaactagttcatctgaaggcagcacggttgatattggagctcccgccgagggagaacagcctgaggttgaacctgaggaatcccttgaacctgaagcctgttttacagaagactgtgtacggaagttcaagtgttgtcagataagcatagaagaaggcaaagggaaactctggtggaatttgaggaaaacatgctataagatagtggagcacaattggttcgaaaccttcattgtcttcatgattctgctgagcagtggggctctggcctttgaagatatatacattgagcagcgaaaaaccattaagaccatgttagaatatgctgacaaggttttcacttacatattcattctggaaatgctgctaaagtgggttgcatatggttttcaagtgtattttaccaatgcctggtgctggctagacttcctgattgttgatgtctcactggttagcttaactgcaaatgccttgggttactcagaacttggtgccatcaaatccctcagaacactaagagctctgaggccactgagagctttgtcccggtttgaaggaatgagggttgttgtaaatgctcttttaggagccattccatctatcatgaatgtacttctggtttgtctgatcttttggctaatattcagtatcatgggagtgaatctctttgctggcaagttttaccattgtattaattacaccactggagagatgtttgatgtaagcgtggtcaacaactacagtgagtgcaaagctctcattgagagcaatcaaactgccaggtggaaaaatgtgaaagtaaactttgataacgtaggacttggatatctgtctctacttcaagtagccacgtttaagggatggatggatattatgtatgcagctgttgattcacgaaatgtagaattacaacccaagtatgaagacaacctgtacatgtatctttattttgtcatctttattatttttggttcattctttaccttgaatcttttcattggtgtcatcatagataacttcaaccaacagaaaaagaagtttggaggtcaagacatttttatgacagaagaacagaagaaatactacaatgcaatgaaaaaactgggttcaaagaaaccacaaaaacccatacctcgacctgctaacaaattccaaggaatggtctttgattttgtaaccaaacaagtctttgatatcagcatcatgatcctcatctgccttaacatggtcaccatgatggtggaaaccgatgaccagagtcaagaaatgacaaacattctgtactggattaatctggtgtttattgttctgttcactggagaatgtgtgctgaaactgatctctcttcgttactactatttcactattggatggaatatttttgattttgtggtggtcattctctccattgtaggaatgtttctggctgaactgatagaaaagtattttgtgtcccctaccctgttccgagtgatccgtcttgccaggattggccgaatcctacgtctgatcaaaggagcaaaggggatccgcacgctgctctttgctttgatgatgtcccttcctgcgttgtttaacatcggcctccttcttttcctggtcatgttcatctacgccatctttgggatgtccaattttgcctatgttaagagggaagttgggatcgatgacatgttcaactttgagacctttggcaacagcatgatctgcctgttccaaattacaacctctgctggctgggatggattgctagcacctattcttaatagtggacctccagactgtgaccctgacaaagatcaccctggaagctcagttaaaggagactgtgggaacccatctgttgggattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcgacccagtttatagagtttgccaaactttctgattttgcagatgccctggatcctcctcttctcatagcaaaacccaacaaagtccagctcattgccatggatctgcccatggtgagtggtgaccggatccactgtcttgacatcttatttgcttttacaaagcgtgttttgggtgagagtggagagatggatgcccttcgaatacagatggaagagcgattcatggcatcaaacccctccaaagtctcttatgagcccattacgaccacgttgaaacgcaaacaagaggaggtgtctgctattattatccagagggcttacagacgctacctcttgaagcaaaaagttaaaaaggtatcaagtatatacaagaaagacaaaggcaaagaatgtgatggaacacccatcaaagaagatactctcattgataaactgaatgagaattcaactccagagaaaaccgatatgacgccttccaccacgtctccaccctcgtatgatagtgtgaccaaaccagaaaaagaaaaatttgaaaaagacaaatcagaaaaggaagacaaagggaaagatatcagggaaagtaaaaagtaaaaagaaaccaagaattttccattttgtgatcaattgtttacagcccgtgatggtgatgtgtttgtgtcaacaggactcccacaggaggtctatgccaaactgactgtttttacaaatgtatacttaaggtcagtgcctataacaagacagagacctctggtcagcaaactggaactcagtaaactggagaaatagtatcgatgggaggtttctattttcacaaccagctgacactgctgaagagcagaggcgtaatggctactcagacgataggaaccaatttaaaggggggagggaagttaaatttttatgtaaattcaacatgtgacacttgataatagtaattgtcaccagtgtttatgttttaactgccacacctgccatatttttacaaaacgtgtgctgtgaatttatcacttttctttttaattcacaggttgtttactattatatgtgactatttttgtaaatgggtttgtgtttggggagagggattaaagggagggaattctacatttctctattgtattgtataactggatatattttaaatggaggcatgctgcaattctcattcacacataaaaaaatcacatcacaaaagggaagagtttacttcttgtttcaggatgtttttagatttttgaggtgcttaaatagctattcgtatttttaaggtgtctcatccagaaaaaatttaatgtgcctgtaaatgttccatagaatcacaagcattaaagagttgttttatttttacataacccattaaatgtacatgtatatatgtatatatgtatatgtgcgtgtatatacatatatatgtatacacacatgcacacacagagatatacacataccattacattgtcattcacagtcccagcagcatgactatcacatttttgataagtgtcctttggcataaaataaaaatatcctatcagtcctttctaagaagcctgaattgaccaaaaaacatccccaccaccactttataaagttgattctgctttatcctgcagtattgtttagccatcttctgctcttggtaaggttgacatagtatatgtcaatttaaaaaataaaagtctgctttgtaaatagtaattttacccagtggtgcatgtttgagcaaacaaaaatgatgatttaagcacactacttattgcatcaaatatgtaccacagtaagtatagtttgcaagctttcaacaggtaatatgatgtaattggttccattatagtttgaagctgtcactgctgcatgtttatcttgcctatgctgctgtatcttattccttccactgttcagaagtctaatatgggaagccatatatcagtggtaaagtgaagcaaattgttctaccaagacctcattcttcatgtcattaagcaataggttgcagcaaacaaggaagagcttcttgctttttattcttccaaccttaattgaacactcaatgatgaaaagcccgactgtacaaacatgttgcaagctgcttaaatctgtttaaaatatatggttagagttttctaagaaaatataaatactgtaaaaagttcattttattttatttttcagccttttgtacgtaaaatgagaaattaaaagtatcttcaggtggatgtcacagtcactattgttagtttctgttcctagcacttttaaattgaagcacttcacaaaataagaagcaaggactaggatgcagtgtaggtttctgcttttttattagtactgtaaacttgcacacatttcaatgtgaaacaaatctcaaactgagttcaatgtttatttgctttcaatagtaatgccttatcattgaaagaggcttaaagaaaaaaaaaatcagctgatactcttggcattgcttgaatccaatgtttccacctagtctttttattcagtaatcatcagtcttttccaatgtttgtttacacagatagatcttattgacccatatggcactagaactgtatcagatataatatgggatcccagctttttttcctctcccacaaaaccaggtagtgaagttatattaccagttacagcaaaatactttgtgtttcacaagcaacaataaatgtagattctttatactgaagctattgacttgtagtgtgttggtgaaatgcatgcaggaaaatgctgttaccataaagaacggtaaaccacattacaatcaagccaaaagaataaaggtttcgcttttgtttttgtatttaattgttgtctttgtttctatctttgaaatgccatttaaaggtagatttctatcatgtaaaaataatctatctgaaaaacaaatgtaaagaacacacattaattactataattcatctttcaattttttcatggaatggaagttaattaagaagagtgtattggataactactttaatattggccaaaaagctagatatggcatcaggtagactagtggaaagttacaaaaattaataaaaaattgactaaca

SEQ ID NO: 3 - DNA, 20 bp, synthetic construct
  source 1..20: mol_type = other DNA
  modified_base 1: mod_base = OTHER; note = 2'-O-methoxyethyl modified 5-methyl cytosine with phosphorothioate internucleoside linkage
  modified_base 2: mod_base = OTHER; note = 2'-O-methoxyethyl modified 5-methyl cytosine
  modified_base 3: mod_base = OTHER; note = 2'-O-methoxyethyl modified nucleotide
  modified_base 4: mod_base = OTHER; note = 2'-O-methoxyethyl modified 5-methyl cytosine
  modified_base 5: mod_base = OTHER; note = 2'-O-methoxyethyl modified nucleotide
  modified_base 6: mod_base = OTHER; note = 2'-O-methoxyethyl modified nucleotide
  modified_base 7: mod_base = OTHER; note = 5-methyl cytosine with phosphorothioate internucleoside linkage
  modified_base 8: mod_base = OTHER; note = Nucleotide with phosphorothioate internucleoside linkage
  modified_base 9: mod_base = OTHER; note = Nucleotide with phosphorothioate internucleoside linkage
  modified_base 10: mod_base = OTHER; note = Nucleotide with phosphorothioate internucleoside linkage
  modified_base 11: mod_base = OTHER; note = Nucleotide with phosphorothioate internucleoside linkage
  modified_base 12: mod_base = OTHER; note = Nucleotide with phosphorothioate internucleoside linkage
  modified_base 13: mod_base = OTHER; note = Nucleotide with phosphorothioate internucleoside linkage
  modified_base 14: mod_base = OTHER; note = Nucleotide with phosphorothioate internucleoside linkage
  modified_base 15: mod_base = OTHER; note = Nucleotide with phosphorothioate internucleoside linkage
  modified_base 16: mod_base = OTHER; note = 5-methyl cytosine with phosphorothioate internucleoside linkage
  modified_base 17: mod_base = OTHER; note = 2'-O-methoxyethyl modified nucleotide
  modified_base 18: mod_base = OTHER; note = 2'-O-methoxyethyl modified nucleotide with phosphorothioate internucleoside linkage
  modified_base 19: mod_base = OTHER; note = 2'-O-methoxyethyl modified 5-methyl cytosine with phosphorothioate internucleoside linkage
  modified_base 20: mod_base = OTHER; note = 2'-O-methoxyethyl modified nucleotide
ccacgacatatttttctaca

SEQ ID NO: 4 - DNA, 8776 bp, Homo sapiens
  source 1..8776: mol_type = genomic DNA
aacagacattgggtaccatcgaatgactgtcagaacagaaagctaaggcaaaggagggaggatgctgtggtcatcctttcttgtttttttcttctttaatgaggatagagcacatgtgagattttactttctactccagtaaaaattctgaagaattgcattggagactgttatattcaacacatacgtggattctgtgttatgatttacatttttctttatttcagcactttcttatgcaaggagctaaacagtgattaaaggagcaggatgaaaagatggcacagtcagtgctggtaccgccaggacctgacagcttccgcttctttaccagggaatcccttgctgctattgaacaacgcattgcagaagagaaagctaagagacccaaacaggaacgcaaggatgaggatgatgaaaatggcccaaagccaaacagtgacttggaagcaggaaaatctcttccatttatttatggagacattcctccagagatggtgtcagtgcccctggaggatctggacccctactatatcaataagaaaacgtttatagtattgaataaagggaaagcaatctctcgattcagtgccacccctgccctttacattttaactcccttcaaccctattagaaaattagctattaagattttggtacattctttattcaatatgctcattatgtgcacgattcttaccaactgtgtatttatgaccatgagtaaccctccagactggacaaagaatgtggagtatacctttacaggaatttatacttttgaatcacttattaaaatacttgcaaggggcttttgtttagaagatttcacatttttacgggatccatggaattggttggatttcacagtcattacttttgcatatgtgacagagtttgtggacctgggcaatgtctcagcgttgagaacattcagagttctccgagcattgaaaacaatttcagtcattccaggcctgaagaccattgtgggggccctgatccagtcagtgaagaagctttctgatgtcatgatcttgactgtgttctgtctaagcgtgtttgcgctaataggattgcagttgttcatgggcaacctacgaaataaatgtttgcaatggcctccagataattcttcctttgaaataaatatcacttccttctttaacaattcattggatgggaatggtactactttcaataggacagtgagcatatttaactgggatgaatatattgaggataaaagtcacttttattttttagaggggcaaaatgatgctctgctttgtggcaacagctcagatgcaggccagtgtcctgaaggatacatctgtgtgaaggctggtagaaaccccaactatggctacacgagctttgacacctttagttgggcctttttgtccttatttcgtctcatgactcaagacttctgggaaaacctttatcaactgacactacgtgctgctgggaaaacgtacatgatattttttgtgctggtcattttcttgggctcattctatctaataaatttgatcttggctgtggtggccatggcctatgaggaacagaatcaggccacattggaagaggctgaacagaaggaagctgaatttcagcagatgctcgaacagttgaaaaagcaacaagaagaagctcaggcggcagctgcagccgcatctgctgaatcaagagacttcagtggtgctggtgggataggagttttttcagagagttcttcagtagcatctaagttgagctccaaaagtgaaaaagagctgaaaaacagaagaaagaaaaagaaacagaaagaacagtctggagaagaagagaaaaatgacagagtccgaaaatcggaatctgaagacagcataagaagaaaaggtttccgtttttccttggaaggaagtaggctgacatatgaaaagagattttcttctccacaccagtccttactgagcatccgtggctcccttttctctccaagacgcaacagtagggcgagccttttcagcttcagaggtcgagcaaaggacattggctctgagaatgactttgctgatgatgagcacagcacctttgaggacaatgacagccgaagagactctctgttcgtgccgcacagacatggagaacggcgccacagcaatgtcagccaggccagccgtgcctccagggtgctccccatcctgcccatgaatgggaagatgcatagcgctgtggactgcaatggtgtggtctccctggtcgggggcccttctaccctcacatctgctgggcagctcctaccagagggcacaactactgaaacagaaataagaaagagacggtccagttcttatcatgtttccatggatttattggaagatcctacatcaaggcaaagagcaatgagtatagccagtattttgaccaacaccatggaagaacttgaagaatccagacagaaatgcccaccatgctggtataaatttgctaatatgtgtttgatttgggactgttgtaaaccatggttaaaggtgaaacaccttgtcaacctggttgtaatggacccatttgttgacctggccatcaccatctgcattgtcttaaatacactcttcatggctatggagcactatcccatgacggagcagttcagcagtgtactgtctgttggaaacctggtcttcacagggatcttcacagcagaaatgtttctcaagataattgccatggatccatattattactttcaagaaggctggaatatttttgatggttttattgtgagccttagtttaatggaacttggtttggcaaatgtggaaggattgtcagttctccgatcattccggctgctccgagttttcaagttggcaaaatcttggccaactctaaatatgctaattaagatcattggcaattctgtgggggctctaggaaacctcaccttggtattggccatcatcgtcttcatttttgctgtggtcggcatgcagctctttggtaagagctacaaagaatgtgtctgcaagatttccaatgattgtgaactcccacgctggcacatgcatgactttttccactccttcctgatcgtgttccgcgtgctgtgtggagagtggatagagaccatgtgggactgtatggaggtcgctggccaaaccatgtgccttactgtcttcatgatggtcatggtgattggaaatctagtggttctgaacctcttcttggccttgcttttgagttccttcagttctgacaatcttgctgccactgatgatgataacgaaatgaataatctccagattgctgtgggaaggatgcagaaaggaatcgattttgttaaaagaaaaatacgtgaatttattcagaaagcctttgttaggaagcagaaagctttagatgaaattaaaccgcttgaagatctaaataataaaaaagacagctgtatttccaaccataccaccatagaaataggcaaagacctcaattatctcaaagacggaaatggaactactagtggcataggcagcagtgtagaaaaatatgtcgtggatgaaagtgattacatgtcatttataaacaaccctagcctcactgtgacagtaccaattgctgttggagaatctgactttgaaaatttaaatactgaagaattcagcagcgagtcagatatggaggaaagcaaagagaagctaaatgcaactagttcatctgaaggcagcacggttgatattggagctcccgccgagggagaacagcctgaggttgaacctgaggaatcccttgaacctgaagcctgttttacagaagactgtgtacggaagttcaagtgttgtcagataagcatagaagaaggcaaagggaaactctggtggaatttgaggaaaacatgctataagatagtggagcacaattggttcgaaaccttcattgtcttcatgattctgctgagcagtggggctctggcctttgaagatatatacattgagcagcgaaaaaccattaagaccatgttagaatatgctgacaaggttttcacttacatattcattctggaaatgctgctaaagtgggttgcatatggttttcaagtgtattttaccaatgcctggtgctggctagacttcctgattgttgatgtctcactggttagcttaactgcaaatgccttgggttactcagaacttggtgccatcaaatccctcagaacactaagagctctgaggccactgagagctttgtcccggtttgaaggaatgagggttgttgtaaatgctcttttaggagccattccatctatcatgaatgtacttctggtttgtctgatcttttggctaatattcagtatcatgggagtgaatctctttgctggcaagttttaccattgtattaattacaccactggagagatgtttgatgtaagcgtggtcaacaactacagtgagtgcaaagctctcattgagagcaatcaaactgccaggtggaaaaatgtgaaagtaaactttgataacgtaggacttggatatctgtctctacttcaagtagccacgtttaagggatggatggatattatgtatgcagctgttgattcacgaaatgtagaattacaacccaagtatgaagacaacctgtacatgtatctttattttgtcatctttattatttttggttcattctttaccttgaatcttttcattggtgtcatcatagataacttcaaccaacagaaaaagaagtttggaggtcaagacatttttatgacagaagaacagaagaaatactacaatgcaatgaaaaaactgggttcaaagaaaccacaaaaacccatacctcgacctgctaacaaattccaaggaatggtctttgattttgtaaccaaacaagtctttgatatcagcatcatgatcctcatctgccttaacatggtcaccatgatggtggaaaccgatgaccagagtcaagaaatgacaaacattctgtactggattaatctggtgtttattgttctgttcactggagaatgtgtgctgaaactgatctctcttcgttactactatttcactattggatggaatatttttgattttgtggtggtcattctctccattgtaggaatgtttctggctgaactgatagaaaagtattttgtgtcccctaccctgttccgagtgatccgtcttgccaggattggccgaatcctacgtctgatcaaaggagcaaaggggatccgcacgctgctctttgctttgatgatgtcccttcctgcgttgtttaacatcggcctccttcttttcctggtcatgttcatctacgccatctttgggatgtccaattttgcctatgttaagagggaagttgggatcgatgacatgttcaactttgagacctttggcaacagcatgatctgcctgttccaaattacaacctctgctggctgggatggattgctagcacctattcttaatagtggacctccagactgtgaccctgacaaagatcaccctggaagctcagttaaaggagactgtgggaacccatctgttgggattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcgacccagtttatagagtttgccaaactttctgattttgcagatgccctggatcctcctcttctcatagcaaaacccaacaaagtccagctcattgccatggatctgcccatggtgagtggtgaccggatccactgtcttgacatcttatttgcttttacaaagcgtgttttgggtgagagtggagagatggatgcccttcgaatacagatggaagagcgattcatggcatcaaacccctccaaagtctcttatgagcccattacgaccacgttgaaacgcaaacaagaggaggtgtctgctattattatccagagggcttacagacgctacctcttgaagcaaaaagttaaaaaggtatcaagtatatacaagaaagacaaaggcaaagaatgtgatggaacacccatcaaagaagatactctcattgataaactgaatgagaattcaactccagagaaaaccgatatgacgccttccaccacgtctccaccctcgtatgatagtgtgaccaaaccagaaaaagaaaaatttgaaaaagacaaatcagaaaaggaagacaaagggaaagatatcagggaaagtaaaaagtaaaaagaaaccaagaattttccattttgtgatcaattgtttacagcccgtgatggtgatgtgtttgtgtcaacaggactcccacaggaggtctatgccaaactgactgtttttacaaatgtatacttaaggtcagtgcctataacaagacagagacctctggtcagcaaactggaactcagtaaactggagaaatagtatcgatgggaggtttctattttcacaaccagctgacactgctgaagagcagaggcgtaatggctactcagacgataggaaccaatttaaaggggggagggaagttaaatttttatgtaaattcaacatgtgacacttgataatagtaattgtcaccagtgtttatgttttaactgccacacctgccatatttttacaaaacgtgtgctgtgaatttatcacttttctttttaattcacaggttgtttactattatatgtgactatttttgtaaatgggtttgtgtttggggagagggattaaagggagggaattctacatttctctattgtattgtataactggatatattttaaatggaggcatgctgcaattctcattcacacataaaaaaatcacatcacaaaagggaagagtttacttcttgtttcaggatgtttttagatttttgaggtgcttaaatagctattcgtatttttaaggtgtctcatccagaaaaaatttaatgtgcctgtaaatgttccatagaatcacaagcattaaagagttgttttatttttacataacccattaaatgtacatgtatatatgtatatatgtatatgtgcgtgtatatacatatatatgtatacacacatgcacacacagagatatacacataccattacattgtcattcacagtcccagcagcatgactatcacatttttgataagtgtcctttggcataaaataaaaatatcctatcagtcctttctaagaagcctgaattgaccaaaaaacatccccaccaccactttataaagttgattctgctttatcctgcagtattgtttagccatcttctgctcttggtaaggttgacatagtatatgtcaatttaaaaaataaaagtctgctttgtaaatagtaattttacccagtggtgcatgtttgagcaaacaaaaatgatgatttaagcacactacttattgcatcaaatatgtaccacagtaagtatagtttgcaagctttcaacaggtaatatgatgtaattggttccattatagtttgaagctgtcactgctgcatgtttatcttgcctatgctgctgtatcttattccttccactgttcagaagtctaatatgggaagccatatatcagtggtaaagtgaagcaaattgttctaccaagacctcattcttcatgtcattaagcaataggttgcagcaaacaaggaagagcttcttgctttttattcttccaaccttaattgaacactcaatgatgaaaagcccgactgtacaaacatgttgcaagctgcttaaatctgtttaaaatatatggttagagttttctaagaaaatataaatactgtaaaaagttcattttattttatttttcagccttttgtacgtaaaatgagaaattaaaagtatcttcaggtggatgtcacagtcactattgttagtttctgttcctagcacttttaaattgaagcacttcacaaaataagaagcaaggactaggatgcagtgtaggtttctgcttttttattagtactgtaaacttgcacacatttcaatgtgaaacaaatctcaaactgagttcaatgtttatttgctttcaatagtaatgccttatcattgaaagaggcttaaagaaaaaaaaaatcagctgatactcttggcattgcttgaatccaatgtttccacctagtctttttattcagtaatcatcagtcttttccaatgtttgtttacacagatagatcttattgacccatatggcactagaactgtatcagatataatatgggatcccagctttttttcctctcccacaaaaccaggtagtgaagttatattaccagttacagcaaaatactttgtgtttcacaagcaacaataaatgtagattctttatactgaagctattgacttgtagtgtgttggtgaaatgcatgcaggaaaatgctgttaccataaagaacggtaaaccacattacaatcaagccaaaagaataaaggtttcgcttttgtttttgtatttaattgttgtctttgtttctatctttgaaatgccatttaaaggtagatttctatcatgtaaaaataatctatctgaaaaacaaatgtaaagaacacacattaattactataattcatctttcaattttttcatggaatggaagttaattaagaagagtgtattggataactactttaatattggccaaaaagctagatatggcatcaggtagactagtggaaagttacaaaaattaataaaaaattgactaaca

SEQ ID NO: 5 - DNA, 8895 bp, Homo sapiens
  source 1..8895: mol_type = genomic DNA
aacagacattgggtaccatcgaatgactgtcagaacagaaagctaaggcaaaggagggaggatgctgtggtcatcctttcttgtttttttcttctttaatgaggatagagcacatgtgagattttactttctactccagtaaaaattctgaagaattgcattggagactgttatattcaacacatacgtggattctgtgttatgatttacatttttctttatttcaggggtttttctccctttgcttgacacttctctgtcctgacaccttgagaagaaggatgtgtttgcttacccttccgccatgattgtaaatttcctgaggccttcccagccatgcagcactcactttcttatgcaaggagctaaacagtgattaaaggagcaggatgaaaagatggcacagtcagtgctggtaccgccaggacctgacagcttccgcttctttaccagggaatcccttgctgctattgaacaacgcattgcagaagagaaagctaagagacccaaacaggaacgcaaggatgaggatgatgaaaatggcccaaagccaaacagtgacttggaagcaggaaaatctcttccatttatttatggagacattcctccagagatggtgtcagtgcccctggaggatctggacccctactatatcaataagaaaacgtttatagtattgaataaagggaaagcaatctctcgattcagtgccacccctgccctttacattttaactcccttcaaccctattagaaaattagctattaagattttggtacattctttattcaatatgctcattatgtgcacgattcttaccaactgtgtatttatgaccatgagtaaccctccagactggacaaagaatgtggagtatacctttacaggaatttatacttttgaatcacttattaaaatacttgcaaggggcttttgtttagaagatttcacatttttacgggatccatggaattggttggatttcacagtcattacttttgcgtatgtaacagaatttgtaaacctaggcaatgtttcagctcttcgaactttcagagtcttgagagctttgaaaactatttctgtaattccaggcctgaagaccattgtgggggccctgatccagtcagtgaagaagctttctgatgtcatgatcttgactgtgttctgtctaagcgtgtttgcgctaataggattgcagttgttcatgggcaacctacgaaataaatgtttgcaatggcctccagataattcttcctttgaaataaatatcacttccttctttaacaattcattggatgggaatggtactactttcaataggacagtgagcatatttaactgggatgaatatattgaggataaaagtcacttttattttttagaggggcaaaatgatgctctgctttgtggcaacagctcagatgcaggccagtgtcctgaaggatacatctgtgtgaaggctggtagaaaccccaactatggctacacgagctttgacacctttagttgggcctttttgtccttatttcgtctcatgactcaagacttctgggaaaacctttatcaactgacactacgtgctgctgggaaaacgtacatgatattttttgtgctggtcattttcttgggctcattctatctaataaatttgatcttggctgtggtggccatggcctatgaggaacagaatcaggccacattggaagaggctgaacagaaggaagctgaatttcagcagatgctcgaacagttgaaaaagcaacaagaagaagctcaggcggcagctgcagccgcatctgctgaatcaagagacttcagtggtgctggtgggataggagttttttcagagagttcttcagtagcatctaagttgagctccaaaagtgaaaaagagctgaaaaacagaagaaagaaaaagaaacagaaagaacagtctggagaagaagagaaaaatgacagagtccgaaaatcggaatctgaagacagcataagaagaaaaggtttccgtttttccttggaaggaagtaggctgacatatgaaaagagattttcttctccacaccagtccttactgagcatccgtggctcccttttctctccaagacgcaacagtagggcgagccttttcagcttcagaggtcgagcaaaggacattggctctgagaatgactttgctgatgatgagcacagcacctttgaggacaatgacagccgaagagactctctgttcgtgccgcacagacatggagaacggcgccacagcaatgtcagccaggccagccgtgcctccagggtgctccccatcctgcccatgaatgggaagatgcatagcgctgtggactgcaatggtgtggtctccctggtcgggggcccttctaccctcacatctgctgggcagctcctaccagagggcacaactactgaaacagaaataagaaagagacggtccagttcttatcatgtttccatggatttattggaagatcctacatcaaggcaaagagcaatgagtatagccagtattttgaccaacaccatggaagaacttgaagaatccagacagaaatgcccaccatgctggtataaatttgctaatatgtgtttgatttgggactgttgtaaaccatggttaaaggtgaaacaccttgtcaacctggttgtaatggacccatttgttgacctggccatcaccatctgcattgtcttaaatacactcttcatggctatggagcactatcccatgacggagcagttcagcagtgtactgtctgttggaaacctggtcttcacagggatcttcacagcagaaatgtttctcaagataattgccatggatccatattattactttcaagaaggctggaatatttttgatggttttattgtgagccttagtttaatggaacttggtttggcaaatgtggaaggattgtcagttctccgatcattccggctgctccgagttttcaagttggcaaaatcttggccaactctaaatatgctaattaagatcattggcaattctgtgggggctctaggaaacctcaccttggtattggccatcatcgtcttcatttttgctgtggtcggcatgcagctctttggtaagagctacaaagaatgtgtctgcaagatttccaatgattgtgaactcccacgctggcacatgcatgactttttccactccttcctgatcgtgttccgcgtgctgtgtggagagtggatagagaccatgtgggactgtatggaggtcgctggccaaaccatgtgccttactgtcttcatgatggtcatggtgattggaaatctagtggttctgaacctcttcttggccttgcttttgagttccttcagttctgacaatcttgctgccactgatgatgataacgaaatgaataatctccagattgctgtgggaaggatgcagaaaggaatcgattttgttaaaagaaaaatacgtgaatttattcagaaagcctttgttaggaagcagaaagctttagatgaaattaaaccgcttgaagatctaaataataaaaaagacagctgtatttccaaccataccaccatagaaataggcaaagacctcaattatctcaaagacggaaatggaactactagtggcataggcagcagtgtagaaaaatatgtcgtggatgaaagtgattacatgtcatttataaacaaccctagcctcactgtgacagtaccaattgctgttggagaatctgactttgaaaatttaaatactgaagaattcagcagcgagtcagatatggaggaaagcaaagagaagctaaatgcaactagttcatctgaaggcagcacggttgatattggagctcccgccgagggagaacagcctgaggttgaacctgaggaatcccttgaacctgaagcctgttttacagaagactgtgtacggaagttcaagtgttgtcagataagcatagaagaaggcaaagggaaactctggtggaatttgaggaaaacatgctataagatagtggagcacaattggttcgaaaccttcattgtcttcatgattctgctgagcagtggggctctggcctttgaagatatatacattgagcagcgaaaaaccattaagaccatgttagaatatgctgacaaggttttcacttacatattcattctggaaatgctgctaaagtgggttgcatatggttttcaagtgtattttaccaatgcctggtgctggctagacttcctgattgttgatgtctcactggttagcttaactgcaaatgccttgggttactcagaacttggtgccatcaaatccctcagaacactaagagctctgaggccactgagagctttgtcccggtttgaaggaatgagggttgttgtaaatgctcttttaggagccattccatctatcatgaatgtacttctggtttgtctgatcttttggctaatattcagtatcatgggagtgaatctctttgctggcaagttttaccattgtattaattacaccactggagagatgtttgatgtaagcgtggtcaacaactacagtgagtgcaaagctctcattgagagcaatcaaactgccaggtggaaaaatgtgaaagtaaactttgataacgtaggacttggatatctgtctctacttcaagtagccacgtttaagggatggatggatattatgtatgcagctgttgattcacgaaatgtagaattacaacccaagtatgaagacaacctgtacatgtatctttattttgtcatctttattatttttggttcattctttaccttgaatcttttcattggtgtcatcatagataacttcaaccaacagaaaaagaagtttggaggtcaagacatttttatgacagaagaacagaagaaatactacaatgcaatgaaaaaactgggttcaaagaaaccacaaaaacccatacctcgacctgctaacaaattccaaggaatggtctttgattttgtaaccaaacaagtctttgatatcagcatcatgatcctcatctgccttaacatggtcaccatgatggtggaaaccgatgaccagagtcaagaaatgacaaacattctgtactggattaatctggtgtttattgttctgttcactggagaatgtgtgctgaaactgatctctcttcgttactactatttcactattggatggaatatttttgattttgtggtggtcattctctccattgtaggaatgtttctggctgaactgatagaaaagtattttgtgtcccctaccctgttccgagtgatccgtcttgccaggattggccgaatcctacgtctgatcaaaggagcaaaggggatccgcacgctgctctttgctttgatgatgtcccttcctgcgttgtttaacatcggcctccttcttttcctggtcatgttcatctacgccatctttgggatgtccaattttgcctatgttaagagggaagttgggatcgatgacatgttcaactttgagacctttggcaacagcatgatctgcctgttccaaattacaacctctgctggctgggatggattgctagcacctattcttaatagtggacctccagactgtgaccctgacaaagatcaccctggaagctcagttaaaggagactgtgggaacccatctgttgggattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcgacccagtttatagagtttgccaaactttctgattttgcagatgccctggatcctcctcttctcatagcaaaacccaacaaagtccagctcattgccatggatctgcccatggtgagtggtgaccggatccactgtcttgacatcttatttgcttttacaaagcgtgttttgggtgagagtggagagatggatgcccttcgaatacagatggaagagcgattcatggcatcaaacccctccaaagtctcttatgagcccattacgaccacgttgaaacgcaaacaagaggaggtgtctgctattattatccagagggcttacagacgctacctcttgaagcaaaaagttaaaaaggtatcaagtatatacaagaaagacaaaggcaaagaatgtgatggaacacccatcaaagaagatactctcattgataaactgaatgagaattcaactccagagaaaaccgatatgacgccttccaccacgtctccaccctcgtatgatagtgtgaccaaaccagaaaaagaaaaatttgaaaaagacaaatcagaaaaggaagacaaagggaaagatatcagggaaagtaaaaagtaaaaagaaaccaagaattttccattttgtgatcaattgtttacagcccgtgatggtgatgtgtttgtgtcaacaggactcccacaggaggtctatgccaaactgactgtttttacaaatgtatacttaaggtcagtgcctataacaagacagagacctctggtcagcaaactggaactcagtaaactggagaaatagtatcgatgggaggtttctattttcacaaccagctgacactgctgaagagcagaggcgtaatggctactcagacgataggaaccaatttaaaggggggagggaagttaaatttttatgtaaattcaacatgtgacacttgataatagtaattgtcaccagtgtttatgttttaactgccacacctgccatatttttacaaaacgtgtgctgtgaatttatcacttttctttttaattcacaggttgtttactattatatgtgactatttttgtaaatgggtttgtgtttggggagagggattaaagggagggaattctacatttctctattgtattgtataactggatatattttaaatggaggcatgctgcaattctcattcacacataaaaaaatcacatcacaaaagggaagagtttacttcttgtttcaggatgtttttagatttttgaggtgcttaaatagctattcgtatttttaaggtgtctcatccagaaaaaatttaatgtgcctgtaaatgttccatagaatcacaagcattaaagagttgttttatttttacataacccattaaatgtacatgtatatatgtatatatgtatatgtgcgtgtatatacatatatatgtatacacacatgcacacacagagatatacacataccattacattgtcattcacagtcccagcagcatgactatcacatttttgataagtgtcctttggcataaaataaaaatatcctatcagtcctttctaagaagcctgaattgaccaaaaaacatccccaccaccactttataaagttgattctgctttatcctgcagtattgtttagccatcttctgctcttggtaaggttgacatagtatatgtcaatttaaaaaataaaagtctgctttgtaaatagtaattttacccagtggtgcatgtttgagcaaacaaaaatgatgatttaagcacactacttattgcatcaaatatgtaccacagtaagtatagtttgcaagctttcaacaggtaatatgatgtaattggttccattatagtttgaagctgtcactgctgcatgtttatcttgcctatgctgctgtatcttattccttccactgttcagaagtctaatatgggaagccatatatcagtggtaaagtgaagcaaattgttctaccaagacctcattcttcatgtcattaagcaataggttgcagcaaacaaggaagagcttcttgctttttattcttccaaccttaattgaacactcaatgatgaaaagcccgactgtacaaacatgttgcaagctgcttaaatctgtttaaaatatatggttagagttttctaagaaaatataaatactgtaaaaagttcattttattttatttttcagccttttgtacgtaaaatgagaaattaaaagtatcttcaggtggatgtcacagtcactattgttagtttctgttcctagcacttttaaattgaagcacttcacaaaataagaagcaaggactaggatgcagtgtaggtttctgcttttttattagtactgtaaacttgcacacatttcaatgtgaaacaaatctcaaactgagttcaatgtttatttgctttcaatagtaatgccttatcattgaaagaggcttaaagaaaaaaaaaatcagctgatactcttggcattgcttgaatccaatgtttccacctagtctttttattcagtaatcatcagtcttttccaatgtttgtttacacagatagatcttattgacccatatggcactagaactgtatcagatataatatgggatcccagctttttttcctctcccacaaaaccaggtagtgaagttatattaccagttacagcaaaatactttgtgtttcacaagcaacaataaatgtagattctttatactgaagctattgacttgtagtgtgttggtgaaatgcatgcaggaaaatgctgttaccataaagaacggtaaaccacattacaatcaagccaaaagaataaaggtttcgcttttgtttttgtatttaattgttgtctttgtttctatctttgaaatgccatttaaaggtagatttctatcatgtaaaaataatctatctgaaaaacaaatgtaaagaacacacattaattactataattcatctttcaattttttcatggaatggaagttaattaagaagagtgtattggataactactttaatattggccaaaaagctagatatggcatcaggtagactagtggaaagttacaaaaattaataaaaaattgactaaca

SEQ ID NO: 6 - DNA, 8776 bp, Homo sapiens
  source 1..8776: mol_type = genomic DNA
aacagacattgggtaccatcgaatgactgtcagaacagaaagctaaggcaaaggagggaggatgctgtggtcatcctttcttgtttttttcttctttaatgaggatagagcacatgtgagattttactttctactccagtaaaaattctgaagaattgcattggagactgttatattcaacacatacgtggattctgtgttatgatttacatttttctttatttcagcactttcttatgcaaggagctaaacagtgattaaaggagcaggatgaaaagatggcacagtcagtgctggtaccgccaggacctgacagcttccgcttctttaccagggaatcccttgctgctattgaacaacgcattgcagaagagaaagctaagagacccaaacaggaacgcaaggatgaggatgatgaaaatggcccaaagccaaacagtgacttggaagcaggaaaatctcttccatttatttatggagacattcctccagagatggtgtcagtgcccctggaggatctggacccctactatatcaataagaaaacgtttatagtattgaataaagggaaagcaatctctcgattcagtgccacccctgccctttacattttaactcccttcaaccctattagaaaattagctattaagattttggtacattctttattcaatatgctcattatgtgcacgattcttaccaactgtgtatttatgaccatgagtaaccctccagactggacaaagaatgtggagtatacctttacaggaatttatacttttgaatcacttattaaaatacttgcaaggggcttttgtttagaagatttcacatttttacgggatccatggaattggttggatttcacagtcattacttttgcgtatgtaacagaatttgtaaacctaggcaatgtttcagctcttcgaactttcagagtcttgagagctttgaaaactatttctgtaattccaggcctgaagaccattgtgggggccctgatccagtcagtgaagaagctttctgatgtcatgatcttgactgtgttctgtctaagcgtgtttgcgctaataggattgcagttgttcatgggcaacctacgaaataaatgtttgcaatggcctccagataattcttcctttgaaataaatatcacttccttctttaacaattcattggatgggaatggtactactttcaataggacagtgagcatatttaactgggatgaatatattgaggataaaagtcacttttattttttagaggggcaaaatgatgctctgctttgtggcaacagctcagatgcaggccagtgtcctgaaggatacatctgtgtgaaggctggtagaaaccccaactatggctacacgagctttgacacctttagttgggcctttttgtccttatttcgtctcatgactcaagacttctgggaaaacctttatcaactgacactacgtgctgctgggaaaacgtacatgatattttttgtgctggtcattttcttgggctcattctatctaataaatttgatcttggctgtggtggccatggcctatgaggaacagaatcaggccacattggaagaggctgaacagaaggaagctgaatttcagcagatgctcgaacagttgaaaaagcaacaagaagaagctcaggcggcagctgcagccgcatctgctgaatcaagagacttcagtggtgctggtgggataggagttttttcagagagttcttcagtagcatctaagttgagctccaaaagtgaaaaagagctgaaaaacagaagaaagaaaaagaaacagaaagaacagtctggagaagaagagaaaaatgacagagtccgaaaatcggaatctgaagacagcataagaagaaaaggtttccgtttttccttggaaggaagtaggctgacatatgaaaagagattttcttctccacaccagtccttactgagcatccgtggctcccttttctctccaagacgcaacagtagggcgagccttttcagcttcagaggtcgagcaaaggacattggctctgagaatgactttgctgatgatgagcacagcacctttgaggacaatgacagccgaagagactctctgttcgtgccgcacagacatggagaacggcgccacagcaatgtcagccaggccagccgtgcctccagggtgctccccatcctgcccatgaatgggaagatgcatagcgctgtggactgcaatggtgtggtctccctggtcgggggcccttctaccctcacatctgctgggcagctcctaccagagggcacaactactgaaacagaaataagaaagagacggtccagttcttatcatgtttccatggatttattggaagatcctacatcaaggcaaagagcaatgagtatagccagtattttgaccaacaccatggaagaacttgaagaatccagacagaaatgcccaccatgctggtataaatttgctaatatgtgtttgatttgggactgttgtaaaccatggttaaaggtgaaacaccttgtcaacctggttgtaatggacccatttgttgacctggccatcaccatctgcattgtcttaaatacactcttcatggctatggagcactatcccatgacggagcagttcagcagtgtactgtctgttggaaacctggtcttcacagggatcttcacagcagaaatgtttctcaagataattgccatggatccatattattactttcaagaaggctggaatatttttgatggttttattgtgagccttagtttaatggaacttggtttggcaaatgtggaaggattgtcagttctccgatcattccggctgctccgagttttcaagttggcaaaatcttggccaactctaaatatgctaattaagatcattggcaattctgtgggggctctaggaaacctcaccttggtattggccatcatcgtcttcatttttgctgtggtcggcatgcagctctttggtaagagctacaaagaatgtgtctgcaagatttccaatgattgtgaactcccacgctggcacatgcatgactttttccactccttcctgatcgtgttccgcgtgctgtgtggagagtggatagagaccatgtgggactgtatggaggtcgctggccaaaccatgtgccttactgtcttcatgatggtcatggtgattggaaatctagtggttctgaacctcttcttggccttgcttttgagttccttcagttctgacaatcttgctgccactgatgatgataacgaaatgaataatctccagattgctgtgggaaggatgcagaaaggaatcgattttgttaaaagaaaaatacgtgaatttattcagaaagcctttgttaggaagcagaaagctttagatgaaattaaaccgcttgaagatctaaataataaaaaagacagctgtatttccaaccataccaccatagaaataggcaaagacctcaattatctcaaagacggaaatggaactactagtggcataggcagcagtgtagaaaaatatgtcgtggatgaaagtgattacatgtcatttataaacaaccctagcctcactgtgacagtaccaattgctgttggagaatctgactttgaaaatttaaatactgaagaattcagcagcgagtcagatatggaggaaagcaaagagaagctaaatgcaactagttcatctgaaggcagcacggttgatattggagctcccgccgagggagaacagcctgaggttgaacctgaggaatcccttgaacctgaagcctgttttacagaagactgtgtacggaagttcaagtgttgtcagataagcatagaagaaggcaaagggaaactctggtggaatttgaggaaaacatgctataagatagtggagcacaattggttcgaaaccttcattgtcttcatgattctgctgagcagtggggctctggcctttgaagatatatacattgagcagcgaaaaaccattaagaccatgttagaatatgctgacaaggttttcacttacatattcattctggaaatgctgctaaagtgggttgcatatggttttcaagtgtattttaccaatgcctggtgctggctagacttcctgattgttgatgtctcactggttagcttaactgcaaatgccttgggttactcagaacttggtgccatcaaatccctcagaacactaagagctctgaggccactgagagctttgtcccggtttgaaggaatgagggttgttgtaaatgctcttttaggagccattccatctatcatgaatgtacttctggtttgtctgatcttttggctaatattcagtatcatgggagtgaatctctttgctggcaagttttaccattgtattaattacaccactggagagatgtttgatgtaagcgtggtcaacaactacagtgagtgcaaagctctcattgagagcaatcaaactgccaggtggaaaaatgtgaaagtaaactttgataacgtaggacttggatatctgtctctacttcaagtagccacgtttaagggatggatggatattatgtatgcagctgttgattcacgaaatgtagaattacaacccaagtatgaagacaacctgtacatgtatctttattttgtcatctttattatttttggttcattctttaccttgaatcttttcattggtgtcatcatagataacttcaaccaacagaaaaagaagtttggaggtcaagacatttttatgacagaagaacagaagaaatactacaatgcaatgaaaaaactgggttcaaagaaaccacaaaaacccatacctcgacctgctaacaaattccaaggaatggtctttgattttgtaaccaaacaagtctttgatatcagcatcatgatcctcatctgccttaacatggtcaccatgatggtggaaaccgatgaccagagtcaagaaatgacaaacattctgtactggattaatctggtgtttattgttctgttcactggagaatgtgtgctgaaactgatctctcttcgttactactatttcactattggatggaatatttttgattttgtggtggtcattctctccattgtaggaatgtttctggctgaactgatagaaaagtattttgtgtcccctaccctgttccgagtgatccgtcttgccaggattggccgaatcctacgtctgatcaaaggagcaaaggggatccgcacgctgctctttgctttgatgatgtcccttcctgcgttgtttaacatcggcctccttcttttcctggtcatgttcatctacgccatctttgggatgtccaattttgcctatgttaagagggaagttgggatcgatgacatgttcaactttgagacctttggcaacagcatgatctgcctgttccaaattacaacctctgctggctgggatggattgctagcacctattcttaatagtggacctccagactgtgaccctgacaaagatcaccctggaagctcagttaaaggagactgtgggaacccatctgttgggattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcgacccagtttatagagtttgccaaactttctgattttgcagatgccctggatcctcctcttctcatagcaaaacccaacaaagtccagctcattgccatggatctgcccatggtgagtggtgaccggatccactgtcttgacatcttatttgcttttacaaagcgtgttttgggtgagagtggagagatggatgcccttcgaatacagatggaagagcgattcatggcatcaaacccctccaaagtctcttatgagcccattacgaccacgttgaaacgcaaacaagaggaggtgtctgctattattatccagagggcttacagacgctacctcttgaagcaaaaagttaaaaaggtatcaagtatatacaagaaagacaaaggcaaagaatgtgatggaacacccatcaaagaagatactctcattgataaactgaatgagaattcaactccagagaaaaccgatatgacgccttccaccacgtctccaccctcgtatgatagtgtgaccaaaccagaaaaagaaaaatttgaaaaagacaaatcagaaaaggaagacaaagggaaagatatcagggaaagtaaaaagtaaaaagaaaccaagaattttccattttgtgatcaattgtttacagcccgtgatggtgatgtgtttgtgtcaacaggactcccacaggaggtctatgccaaactgactgtttttacaaatgtatacttaaggtcagtgcctataacaagacagagacctctggtcagcaaactggaactcagtaaactggagaaatagtatcgatgggaggtttctattttcacaaccagctgacactgctgaagagcagaggcgtaatggctactcagacgataggaaccaatttaaaggggggagggaagttaaatttttatgtaaattcaacatgtgacacttgataatagtaattgtcaccagtgtttatgttttaactgccacacctgccatatttttacaaaacgtgtgctgtgaatttatcacttttctttttaattcacaggttgtttactattatatgtgactatttttgtaaatgggtttgtgtttggggagagggattaaagggagggaattctacatttctctattgtattgtataactggatatattttaaatggaggcatgctgcaattctcattcacacataaaaaaatcacatcacaaaagggaagagtttacttcttgtttcaggatgtttttagatttttgaggtgcttaaatagctattcgtatttttaaggtgtctcatccagaaaaaatttaatgtgcctgtaaatgttccatagaatcacaagcattaaagagttgttttatttttacataacccattaaatgtacatgtatatatgtatatatgtatatgtgcgtgtatatacatatatatgtatacacacatgcacacacagagatatacacataccattacattgtcattcacagtcccagcagcatgactatcacatttttgataagtgtcctttggcataaaataaaaatatcctatcagtcctttctaagaagcctgaattgaccaaaaaacatccccaccaccactttataaagttgattctgctttatcctgcagtattgtttagccatcttctgctcttggtaaggttgacatagtatatgtcaatttaaaaaataaaagtctgctttgtaaatagtaattttacccagtggtgcatgtttgagcaaacaaaaatgatgatttaagcacactacttattgcatcaaatatgtaccacagtaagtatagtttgcaagctttcaacaggtaatatgatgtaattggttccattatagtttgaagctgtcactgctgcatgtttatcttgcctatgctgctgtatcttattccttccactgttcagaagtctaatatgggaagccatatatcagtggtaaagtgaagcaaattgttctaccaagacctcattcttcatgtcattaagcaataggttgcagcaaacaaggaagagcttcttgctttttattcttccaaccttaattgaacactcaatgatgaaaagcccgactgtacaaacatgttgcaagctgcttaaatctgtttaaaatatatggttagagttttctaagaaaatataaatactgtaaaaagttcattttattttatttttcagccttttgtacgtaaaatgagaaattaaaagtatcttcaggtggatgtcacagtcactattgttagtttctgttcctagcacttttaaattgaagcacttcacaaaataagaagcaaggactaggatgcagtgtaggtttctgcttttttattagtactgtaaacttgcacacatttcaatgtgaaacaaatctcaaactgagttcaatgtttatttgctttcaatagtaatgccttatcattgaaagaggcttaaagaaaaaaaaaatcagctgatactcttggcattgcttgaatccaatgtttccacctagtctttttattcagtaatcatcagtcttttccaatgtttgtttacacagatagatcttattgacccatatggcactagaactgtatcagatataatatgggatcccagctttttttcctctcccacaaaaccaggtagtgaagttatattaccagttacagcaaaatactttgtgtttcacaagcaacaataaatgtagattctttatactgaagctattgacttgtagtgtgttggtgaaatgcatgcaggaaaatgctgttaccataaagaacggtaaaccacattacaatcaagccaaaagaataaaggtttcgcttttgtttttgtatttaattgttgtctttgtttctatctttgaaatgccatttaaaggtagatttctatcatgtaaaaataatctatctgaaaaacaaatgtaaagaacacacattaattactataattcatctttcaattttttcatggaatggaagttaattaagaagagtgtattggataactactttaatattggccaaaaagctagatatggcatcaggtagactagtggaaagttacaaaaattaataaaaaattgactaaca

SEQ ID NO: 7 - DNA, 8604 bp, Homo sapiens
  source 1..8604: mol_type = genomic DNA
atagcagtaacacaattcacctctagtgtgaacatatcaggatggcatagaccagcactttcttatgcaaggagctaaacagtgattaaaggagcaggatgaaaagatggcacagtcagtgctggtaccgccaggacctgacagcttccgcttctttaccagggaatcccttgctgctattgaacaacgcattgcagaagagaaagctaagagacccaaacaggaacgcaaggatgaggatgatgaaaatggcccaaagccaaacagtgacttggaagcaggaaaatctcttccatttatttatggagacattcctccagagatggtgtcagtgcccctggaggatctggacccctactatatcaataagaaaacgtttatagtattgaataaagggaaagcaatctctcgattcagtgccacccctgccctttacattttaactcccttcaaccctattagaaaattagctattaagattttggtacattctttattcaatatgctcattatgtgcacgattcttaccaactgtgtatttatgaccatgagtaaccctccagactggacaaagaatgtggagtatacctttacaggaatttatacttttgaatcacttattaaaatacttgcaaggggcttttgtttagaagatttcacatttttacgggatccatggaattggttggatttcacagtcattacttttgcatatgtgacagagtttgtggacctgggcaatgtctcagcgttgagaacattcagagttctccgagcattgaaaacaatttcagtcattccaggcctgaagaccattgtgggggccctgatccagtcagtgaagaagctttctgatgtcatgatcttgactgtgttctgtctaagcgtgtttgcgctaataggattgcagttgttcatgggcaacctacgaaataaatgtttgcaatggcctccagataattcttcctttgaaataaatatcacttccttctttaacaattcattggatgggaatggtactactttcaataggacagtgagcatatttaactgggatgaatatattgaggataaaagtcacttttattttttagaggggcaaaatgatgctctgctttgtggcaacagctcagatgcaggccagtgtcctgaaggatacatctgtgtgaaggctggtagaaaccccaactatggctacacgagctttgacacctttagttgggcctttttgtccttatttcgtctcatgactcaagacttctgggaaaacctttatcaactgacactacgtgctgctgggaaaacgtacatgatattttttgtgctggtcattttcttgggctcattctatctaataaatttgatcttggctgtggtggccatggcctatgaggaacagaatcaggccacattggaagaggctgaacagaaggaagctgaatttcagcagatgctcgaacagttgaaaaagcaacaagaagaagctcaggcggcagctgcagccgcatctgctgaatcaagagacttcagtggtgctggtgggataggagttttttcagagagttcttcagtagcatctaagttgagctccaaaagtgaaaaagagctgaaaaacagaagaaagaaaaagaaacagaaagaacagtctggagaagaagagaaaaatgacagagtccgaaaatcggaatctgaagacagcataagaagaaaaggtttccgtttttccttggaaggaagtaggctgacatatgaaaagagattttcttctccacaccagtccttactgagcatccgtggctcccttttctctccaagacgcaacagtagggcgagccttttcagcttcagaggtcgagcaaaggacattggctctgagaatgactttgctgatgatgagcacagcacctttgaggacaatgacagccgaagagactctctgttcgtgccgcacagacatggagaacggcgccacagcaatgtcagccaggccagccgtgcctccagggtgctccccatcctgcccatgaatgggaagatgcatagcgctgtggactgcaatggtgtggtctccctggtcgggggcccttctaccctcacatctgctgggcagctcctaccagagggcacaactactgaaacagaaataagaaagagacggtccagttcttatcatgtttccatggatttattggaagatcctacatcaaggcaaagagcaatgagtatagccagtattttgaccaacaccatggaagaacttgaagaatccagacagaaatgcccaccatgctggtataaatttgctaatatgtgtttgatttgggactgttgtaaaccatggttaaaggtgaaacaccttgtcaacctggttgtaatggacccatttgttgacctggccatcaccatctgcattgtcttaaatacactcttcatggctatggagcactatcccatgacggagcagttcagcagtgtactgtctgttggaaacctggtcttcacagggatcttcacagcagaaatgtttctcaagataattgccatggatccatattattactttcaagaaggctggaatatttttgatggttttattgtgagccttagtttaatggaacttggtttggcaaatgtggaaggattgtcagttctccgatcattccggctgctccgagttttcaagttggcaaaatcttggccaactctaaatatgctaattaagatcattggcaattctgtgggggctctaggaaacctcaccttggtattggccatcatcgtcttcatttttgctgtggtcggcatgcagctctttggtaagagctacaaagaatgtgtctgcaagatttccaatgattgtgaactcccacgctggcacatgcatgactttttccactccttcctgatcgtgttccgcgtgctgtgtggagagtggatagagaccatgtgggactgtatggaggtcgctggccaaaccatgtgccttactgtcttcatgatggtcatggtgattggaaatctagtggttctgaacctcttcttggccttgcttttgagttccttcagttctgacaatcttgctgccactgatgatgataacgaaatgaataatctccagattgctgtgggaaggatgcagaaaggaatcgattttgttaaaagaaaaatacgtgaatttattcagaaagcctttgttaggaagcagaaagctttagatgaaattaaaccgcttgaagatctaaataataaaaaagacagctgtatttccaaccataccaccatagaaataggcaaagacctcaattatctcaaagacggaaatggaactactagtggcataggcagcagtgtagaaaaatatgtcgtggatgaaagtgattacatgtcatttataaacaaccctagcctcactgtgacagtaccaattgctgttggagaatctgactttgaaaatttaaatactgaagaattcagcagcgagtcagatatggaggaaagcaaagagaagctaaatgcaactagttcatctgaaggcagcacggttgatattggagctcccgccgagggagaacagcctgaggttgaacctgaggaatcccttgaacctgaagcctgttttacagaagactgtgtacggaagttcaagtgttgtcagataagcatagaagaaggcaaagggaaactctggtggaatttgaggaaaacatgctataagatagtggagcacaattggttcgaaaccttcattgtcttcatgattctgctgagcagtggggctctggcctttgaagatatatacattgagcagcgaaaaaccattaagaccatgttagaatatgctgacaaggttttcacttacatattcattctggaaatgctgctaaagtgggttgcatatggttttcaagtgtattttaccaatgcctggtgctggctagacttcctgattgttgatgtctcactggttagcttaactgcaaatgccttgggttactcagaacttggtgccatcaaatccctcagaacactaagagctctgaggccactgagagctttgtcccggtttgaaggaatgagggttgttgtaaatgctcttttaggagccattccatctatcatgaatgtacttctggtttgtctgatcttttggctaatattcagtatcatgggagtgaatctctttgctggcaagttttaccattgtattaattacaccactggagagatgtttgatgtaagcgtggtcaacaactacagtgagtgcaaagctctcattgagagcaatcaaactgccaggtggaaaaatgtgaaagtaaactttgataacgtaggacttggatatctgtctctacttcaagtagccacgtttaagggatggatggatattatgtatgcagctgttgattcacgaaatgtagaattacaacccaagtatgaagacaacctgtacatgtatctttattttgtcatctttattatttttggttcattctttaccttgaatcttttcattggtgtcatcatagataacttcaaccaacagaaaaagaagtttggaggtcaagacatttttatgacagaagaacagaagaaatactacaatgcaatgaaaaaactgggttcaaagaaaccacaaaaacccatacctcgacctgctaacaaattccaaggaatggtctttgattttgtaaccaaacaagtctttgatatcagcatcatgatcctcatctgccttaacatggtcaccatgatggtggaaaccgatgaccagagtcaagaaatgacaaacattctgtactggattaatctggtgtttattgttctgttcactggagaatgtgtgctgaaactgatctctcttcgttactactatttcactattggatggaatatttttgattttgtggtggtcattctctccattgtaggaatgtttctggctgaactgatagaaaagtattttgtgtcccctaccctgttccgagtgatccgtcttgccaggattggccgaatcctacgtctgatcaaaggagcaaaggggatccgcacgctgctctttgctttgatgatgtcccttcctgcgttgtttaacatcggcctccttcttttcctggtcatgttcatctacgccatctttgggatgtccaattttgcctatgttaagagggaagttgggatcgatgacatgttcaactttgagacctttggcaacagcatgatctgcctgttccaaattacaacctctgctggctgggatggattgctagcacctattcttaatagtggacctccagactgtgaccctgacaaagatcaccctggaagctcagttaaaggagactgtgggaacccatctgttgggattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcgacccagtttatagagtttgccaaactttctgattttgcagatgccctggatcctcctcttctcatagcaaaacccaacaaagtccagctcattgccatggatctgcccatggtgagtggtgaccggatccactgtcttgacatcttatttgcttttacaaagcgtgttttgggtgagagtggagagatggatgcccttcgaatacagatggaagagcgattcatggcatcaaacccctccaaagtctcttatgagcccattacgaccacgttgaaacgcaaacaagaggaggtgtctgctattattatccagagggcttacagacgctacctcttgaagcaaaaagttaaaaaggtatcaagtatatacaagaaagacaaaggcaaagaatgtgatggaacacccatcaaagaagatactctcattgataaactgaatgagaattcaactccagagaaaaccgatatgacgccttccaccacgtctccaccctcgtatgatagtgtgaccaaaccagaaaaagaaaaatttgaaaaagacaaatcagaaaaggaagacaaagggaaagatatcagggaaagtaaaaagtaaaaagaaaccaagaattttccattttgtgatcaattgtttacagcccgtgatggtgatgtgtttgtgtcaacaggactcccacaggaggtctatgccaaactgactgtttttacaaatgtatacttaaggtcagtgcctataacaagacagagacctctggtcagcaaactggaactcagtaaactggagaaatagtatcgatgggaggtttctattttcacaaccagctgacactgctgaagagcagaggcgtaatggctactcagacgataggaaccaatttaaaggggggagggaagttaaatttttatgtaaattcaacatgtgacacttgataatagtaattgtcaccagtgtttatgttttaactgccacacctgccatatttttacaaaacgtgtgctgtgaatttatcacttttctttttaattcacaggttgtttactattatatgtgactatttttgtaaatgggtttgtgtttggggagagggattaaagggagggaattctacatttctctattgtattgtataactggatatattttaaatggaggcatgctgcaattctcattcacacataaaaaaatcacatcacaaaagggaagagtttacttcttgtttcaggatgtttttagatttttgaggtgcttaaatagctattcgtatttttaaggtgtctcatccagaaaaaatttaatgtgcctgtaaatgttccatagaatcacaagcattaaagagttgttttatttttacataacccattaaatgtacatgtatatatgtatatatgtatatgtgcgtgtatatacatatatatgtatacacacatgcacacacagagatatacacataccattacattgtcattcacagtcccagcagcatgactatcacatttttgataagtgtcctttggcataaaataaaaatatcctatcagtcctttctaagaagcctgaattgaccaaaaaacatccccaccaccactttataaagttgattctgctttatcctgcagtattgtttagccatcttctgctcttggtaaggttgacatagtatatgtcaatttaaaaaataaaagtctgctttgtaaatagtaattttacccagtggtgcatgtttgagcaaacaaaaatgatgatttaagcacactacttattgcatcaaatatgtaccacagtaagtatagtttgcaagctttcaacaggtaatatgatgtaattggttccattatagtttgaagctgtcactgctgcatgtttatcttgcctatgctgctgtatcttattccttccactgttcagaagtctaatatgggaagccatatatcagtggtaaagtgaagcaaattgttctaccaagacctcattcttcatgtcattaagcaataggttgcagcaaacaaggaagagcttcttgctttttattcttccaaccttaattgaacactcaatgatgaaaagcccgactgtacaaacatgttgcaagctgcttaaatctgtttaaaatatatggttagagttttctaagaaaatataaatactgtaaaaagttcattttattttatttttcagccttttgtacgtaaaatgagaaattaaaagtatcttcaggtggatgtcacagtcactattgttagtttctgttcctagcacttttaaattgaagcacttcacaaaataagaagcaaggactaggatgcagtgtaggtttctgcttttttattagtactgtaaacttgcacacatttcaatgtgaaacaaatctcaaactgagttcaatgtttatttgctttcaatagtaatgccttatcattgaaagaggcttaaagaaaaaaaaaatcagctgatactcttggcattgcttgaatccaatgtttccacctagtctttttattcagtaatcatcagtcttttccaatgtttgtttacacagatagatcttattgacccatatggcactagaactgtatcagatataatatgggatcccagctttttttcctctcccacaaaaccaggtagtgaagttatattaccagttacagcaaaatactttgtgtttcacaagcaacaataaatgtagattctttatactgaagctattgacttgtagtgtgttggtgaaatgcatgcaggaaaatgctgttaccataaagaacggtaaaccacattacaatcaagccaaaagaataaaggtttcgcttttgtttttgtatttaattgttgtctttgtttctatctttgaaatgccatttaaaggtagatttctatcatgtaaaaataatctatctgaaaaacaaatgtaaagaacacacattaattactataattcatctttcaattttttcatggaatggaagttaattaagaagagtgtattggataactactttaatattggccaaaaagctagatatggcatcaggtagactagtggaaagttacaaaaattaataaaaaattgactaaca

SEQ ID NO: 8 - protein, 2005 aa, Homo sapiens; identical to SEQ ID NO: 1

SEQ ID NO: 9 - protein, 2005 aa, Homo sapiens
MAQSVLVPPGPDSFRFFTRESLAAIEQRIAEEKAKRPKQERKDEDDENGPKPNSDLEAGKSLPFIYGDIPPEMVSVPLEDLDPYYINKKTFIVLNKGKAISRFSATPALYILTPFNPIRKLAIKILVHSLFNMLIMCTILTNCVFMTMSNPPDWTKNVEYTFTGIYTFESLIKILARGFCLEDFTFLRDPWNWLDFTVITFAYVTEFVNLGNVSALRTFRVLRALKTISVIPGLKTIVGALIQSVKKLSDVMILTVFCLSVFALIGLQLFMGNLRNKCLQWPPDNSSFEINITSFFNNSLDGNGTTFNRTVSIFNWDEYIEDKSHFYFLEGQNDALLCGNSSDAGQCPEGYICVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMLEQLKKQQEEAQAAAAAASAESRDFSGAGGIGVFSESSSVASKLSSKSEKELKNRRKKKKQKEQSGEEEKNDRVRKSESEDSIRRKGFRFSLEGSRLTYEKRFSSPHQSLLSIRGSLFSPRRNSRASLFSFRGRAKDIGSENDFADDEHSTFEDNDSRRDSLFVPHRHGERRHSNVSQASRASRVLPILPMNGKMHSAVDCNGVVSLVGGPSTLTSAGQLLPEGTTTETEIRKRRSSSYHVSMDLLEDPTSRQRAMSIASILTNTMEELEESRQKCPPCWYKFANMCLIWDCCKPWLKVKHLVNLVVMDPFVDLAITICIVLNTLFMAMEHYPMTEQFSSVLSVGNLVFTGIFTAEMFLKIIAMDPYYYFQEGWNIFDGFIVSLSLMELGLANVEGLSVLRSFRLLRVFKLAKSWPTLNMLIKIIGNSVGALGNLTLVLAIIVFIFAVVGMQLFGKSYKECVCKISNDCELPRWHMHDFFHSFLIVFRVLCGEWIETMWDCMEVAGQTMCLTVFMMVMVIGNLVVLNLFLALLLSSFSSDNLAATDDDNEMNNLQIAVGRMQKGIDFVKRKIREFIQKAFVRKQKALDEIKPLEDLNNKKDSCISNHTTIEIGKDLNYLKDGNGTTSGIGSSVEKYVVDESDYMSFINNPSLTVTVPIAVGESDFENLNTEEFSSESDMEESKEKLNATSSSEGSTVDIGAPAEGEQPEVEPEESLEPEACFTEDCVRKFKCCQISIEEGKGKLWWNLRKTCYKIVEHNWFETFIVFMILLSSGALAFEDIYIEQRKTIKTMLEYADKVFTYIFILEMLLKWVAYGFQVYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINYTTGEMFDVSVVNNYSECKALIESNQTARWKNVKVNFDNVGLGYLSLLQVATFKGWMDIMYAAVDSRNVELQPKYEDNLYMYLYFVIFIIFGSFFTLNLFIGVIIDNFNQQKKKFGGQDIFMTEEQKKYYNAMKKLGSKKPQKPIPRPANKFQGMVFDFVTKQVFDISIMILICLNMVTMMVETDDQSQEMTNILYWINLVFIVLFTGECVLKLISLRYYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSGPPDCDPDKDHPGSSVKGDCGNPSVGIFFFVSYIIISFLVVVNMYIAVILENFSVATEESAEPLSEDDFEMFYEVWEKFDPDATQFIEFAKLSDFADALDPPLLIAKPNKVQLIAMDLPMVSGDRIHCLDILFAFTKRVLGESGEMDALRIQMEERFMASNPSKVSYEPITTTLKRKQEEVSAIIIQRAYRRYLLKQKVKKVSSIYKKDKGKECDGTPIKEDTLIDKLNENSTPEKTDMTPSTTSPPSYDSVTKPEKEKFEKDKSEKEDKGKDIRESKK

(19) United States
(12) Patent Application Publication
(10) Pub. No.: US 2025/0092405 A1
(43) Pub. Date: Mar. 20, 2025

(54) METHODS OF TREATMENT OF SCN2A-RELATED DISORDERS

(71) Applicant: Praxis Precision Medicines, Inc., Boston, MA (US)

(72) Inventors: William Motel, Baltimore, MD (US); Alyssa Wyant, Califon, NJ (US); Marjie Marjie, Lexington, MA (US)

(73) Assignee: Praxis Precision Medicines, Inc., Boston, MA (US)

(21) Appl. No.: 18/885,261

(22) Filed: Sep. 13, 2024

Related U.S. Application Data
(60) Provisional application No. 63/689,297, filed on Aug. 30, 2024.
(60) Provisional application No. 63/686,359, filed on Aug. 23, 2024.
(60) Provisional application No. 63/672,118, filed on Jul. 16, 2024.
(60) Provisional application No. 63/651,681, filed on May 24, 2024.
(60) Provisional application No. 63/604,103, filed on Nov. 29, 2023.
(60) Provisional application No. 63/542,017, filed on Oct. 2, 2023.
(60) Provisional application No. 63/538,713, filed on Sep. 15, 2023.

(51) Int. Cl. C12N 15/113; A61P 25/08

(52) U.S. Cl. CPC C12N 15/1138; A61P 25/08; C12N 2310/315; C12N 2310/321; C12N 2310/3341; C12N 2310/341; C12N 2320/34; C12N 2320/35

(57) ABSTRACT

Provided are methods of treating a subject with a SCN2A-related disorder, e.g., Developmental and Epileptic Encephalopathies (DEE), comprising administering to the subject an oligomeric compound. Also provided are methods of reducing frequency of seizures experienced by a subject with a SCN2A-related disorder, comprising administering to the subject an oligomeric compound.

### RELATED APPLICATIONS

This application claims priority to U.S. Provisional Patent Application No. 63/689,297, filed on Aug. 30, 2024; U.S. Provisional Patent Application No. 63/686,359, filed on Aug. 23, 2024; U.S. Provisional Patent Application No. 63/672,118, filed on Jul. 16, 2024; U.S. Provisional Patent Application No. 63/651,681, filed on May 24, 2024; U.S. Provisional Patent Application No. 63/604,103, filed on Nov. 29, 2023; U.S. Provisional Patent Application No. 63/542,017, filed on Oct. 2, 2023; and U.S. Provisional Patent Application No. 63/538,713, filed on Sep. 15, 2023. The entire contents of each of the foregoing applications are hereby incorporated herein by reference.

### SEQUENCE LISTING

The instant application contains a Sequence Listing which has been submitted electronically in XML file format and is hereby incorporated by reference in its entirety. Said XML copy, created on Sep. 13, 2024, is named 137486-09408_SL.xml and is 69,914 bytes in size.

### BACKGROUND

The human gene SCN2A encodes human SCN2A protein, the alpha-1 subunit of the voltage-gated sodium channel NaV1.2. Mutations in SCN2A are associated with a variety of neurodevelopmental and intellectual diseases and disorders, such as Developmental and Epileptic Encephalopathies (DEE), including Early Seizure Onset Epileptic Encephalopathy (EE), Late Seizure Onset Epileptic Encephalopathy, and Benign Familial Neonatal-Infantile Seizures (BFNIS). Mutations in SCN2A are also associated with intellectual disability (ID) and/or autism spectrum disorder (ASD), with or without seizures (Wolff, M., et al., 2019, Epilepsia 60, S59-S67; Sanders, S., et al., 2018, Trends in Neurosciences 41, 442-456; Wolff, M., et al., 2017, Brain 140, 1316-1336). DEEs include a broad range of diseases that include neonatal and early infantile DEE, for example Ohtahara Syndrome and epilepsy with migrating focal seizures of infancy (EIMFS); infantile and childhood DEE, for example West Syndrome and Lennon-Gastaut Syndrome; Dravet Syndrome; Idiopathic/Generic Generalized Epilepsies (IGE/GGE); Temporal Lobe Epilepsy; Myoclonic Astatic Epilepsy (MAE); Migrating Partial Epilepsy of Infancy (MMPSI); and familial hemiplegic migraines, with or without epilepsy (Wolff, M., et al., 2019; Harkin, L. A., et al., 2007, Brain 130, 843-852; Escayg, A., et al., 2010, Epilepsia 51, 1650-1658; Miller I. O, et al., 2007 Nov. 29 [Updated 2019 Apr. 18]. In: Adam M P, Ardinger H H, Pagon R A, et al., editors. GeneReviews® [Internet]. Seattle (WA): University of Washington, Seattle; 1993-2020. Available from: www.ncbi.nlm.nih.gov/books/NBK1318/).

Symptoms and hallmarks associated with DEEs include seizures, hypotonia, sensory integration disorders, motor development delays and dysfunctions, intellectual and cognitive dysfunctions, movement and balance dysfunctions, visual dysfunctions, delayed language and speech, gastrointestinal disorders, neurodevelopmental delays, sleep problems, and sudden unexpected death in epilepsy. Seizures include focal, clonic, tonic, and generalized tonic and clonic seizures, prolonged seizures (often lasting longer than 10 minutes), and frequent seizures (for example, convulsive, myoclonic, absence, focal, obtundation status, and tonic seizures) (Guzzetta, F., 2011, Epilepsia 52:S2, 35-38; Anwar et al., 2019, Cureus 11, e5006, Wolff et al., 2019). Symptoms and hallmarks associated with ID and ASD include motor development delays, delayed social and language milestones, repetitive actions, uncoordinated oral movements, gastrointestinal disorders, sleep problems, and seizures (Wolff et al., 2019).

Currently there is a lack of acceptable options for treating DEEs such as EEs, Late Onset EEs, and BFNIS; and for treating ID and ASD. It is therefore an object herein to provide methods for the treatment of such diseases and disorders.

### SUMMARY OF THE INVENTION

In one aspect, disclosed herein is a method of reducing frequency of seizures experienced by a subject with a SCN2A-related disorder, said method comprising administering to said subject an effective amount of an oligomeric compound, wherein: the SCN2A-related disorder is caused by a gain-of-function mutation in SCN2A gene; the oligomeric compound comprises a 6-10-4 MOE gapmer having a sequence (from 5′ to 3′) of CCACGACATATTTTTCTACA (SEQ ID NO: 3); wherein each of nucleosides 1-6 and 17-20 (from 5′ to 3′) are 2′-MOE nucleosides and each of nucleosides 7-16 are 2′-β-D-deoxynucleosides; wherein the internucleoside linkages between nucleosides 2 to 3, 3 to 4, 4 to 5, 5 to 6, 6 to 7, and 17 to 18 are phosphodiester internucleoside linkages, the internucleoside linkages between nucleosides 1 to 2, 7 to 8, 8 to 9, 9 to 10, 10 to 11, 11 to 12, 12 to 13, 13 to 14, 14 to 15, 15 to 16, 16 to 17, 18 to 19, and 19 to 20 are phosphorothioate internucleoside linkages; and each cytosine is a 5-methyl cytosine.

In some embodiments, the oligomeric compound is administered at a dose of about 0.1 mg to about 20 mg. In some embodiments, the oligomeric compound is administered at a dose of about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.5 mg, about 2 mg, about 2.5 mg, about 3 mg, about 3.5 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg or about 20 mg. In some embodiments, the oligomeric compound is administered at a dose of about 0.5 mg, about 1 mg, about 1.5 mg, about 2 mg, about 2.5 mg, about 3 mg, about 3.5 mg, about 4 mg, about 5 mg, about 8 mg, about 12 mg or about 15 mg. In some embodiments, the oligomeric compound is administered at a dose of about 1 mg. In some embodiments, the oligomeric compound is administered at a dose of about 8 mg.

In some embodiments, the oligomeric compound is administered at a dose of about 0.5 mg to about 8 mg, e.g., about 0.5 mg, about 1 mg, about 1.5 mg, about 2 mg, about 2.5 mg, about 3 mg, about 3.5 mg, about 4 mg, about 5 mg, about 6 mg, about 7 mg or about 8 mg. Is some embodiments, the oligomeric compound is administered at a dose of about 0.5 mg to about 8 mg, e.g., about 0.5 mg, about 1 mg, about 1.5 mg, about 2 mg, about 2.5 mg, about 3 mg, about 3.5 mg, about 4 mg, about 5 mg, about 6 mg, about 7 mg or about 8 mg, every 2-6 weeks, e.g., every 2 weeks, every 3 weeks, every 4 weeks, every 5 weeks or every 6 weeks. Intervals between administrations may be approximate and may be varied by a clinician based upon medical judgment and/or clinical indications such as for example patient travel, availability of medical personnel and drug, illness, or adverse event in a patient.

In some embodiments, the oligomeric compound is administered at a total dose of about 15 mg to about 30 mg over about 20-25 weeks, e.g., about 15 mg, about 20 mg, about 22.5 mg, about 25 mg or about 30 mg over 20 weeks, 21 weeks, 22 weeks, 23 weeks, 24 weeks or 25 weeks. In some embodiments, the oligomeric compound is administered at a total dose of about 22.5 mg over 23 weeks.

In some embodiments, the oligomeric compound is administered once per month. In other embodiments, the oligomeric compound is administered more than once per month. In further embodiments, the oligomeric compound is administered once every two, three, four, five, or six months, or once per year.

In some embodiments, the seizures are selected from the group consisting of focal motor seizures, tonic seizures, generalized tonic-clonic seizures and myoclonic seizures.

In some embodiments, administration of the oligomeric compound results in a decrease in the average number of daily seizures experienced by the subject in a 28-day period, as compared to the average number of daily seizures experienced by the subject prior to administration of the oligomeric compound.

In some embodiments, administration of the oligomeric compound results in a reduction in the number of seizures experienced by the subject in a 28-day period by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, as compared to the number of seizures experienced by the subject prior to administration of the oligomeric compound.

In one aspect, disclosed herein is a method of treating a subject with a SCN2A-related disorder, said method comprising administering to said subject an oligomeric compound at a dose of about 0.1 mg to about 20 mg, wherein: the SCN2A-related disorder is caused by a gain-of-function mutation in SCN2A gene; the oligomeric compound comprises a 6-10-4 MOE gapmer having a sequence (from 5′ to 3′) of CCACGACATATTTTTCTACA (SEQ ID NO: 3); each of nucleosides 1-6 and 17-20 (from 5′ to 3′) are 2′-MOE nucleosides and each of nucleosides 7-16 are 2′-β-D-deoxynucleosides; the internucleoside linkages between nucleosides 2 to 3, 3 to 4, 4 to 5, 5 to 6, 6 to 7, and 17 to 18 are phosphodiester internucleoside linkages, the internucleoside linkages between nucleosides 1 to 2, 7 to 8, 8 to 9, 9 to 10, 10 to 11, 11 to 12, 12 to 13, 13 to 14, 14 to 15, 15 to 16, 16 to 17, 18 to 19, and 19 to 20 are phosphorothioate internucleoside linkages; and each cytosine is a 5-methyl cytosine.

In some embodiments, the oligomeric compound is administered at a dose of about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.5 mg, about 2 mg, about 2.5 mg, about 3 mg, about 3.5 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg or about 20 mg. In some embodiments, the oligomeric compound is administered at a dose of about 0.5 mg, about 1 mg, about 1.5 mg, about 3 mg, about 3.5 mg, about 4 mg, about 5 mg, about 8 mg, about 12 mg or about 15 mg. In some embodiments, the oligomeric compound is administered at a dose of about 1 mg. In some embodiments, the oligomeric compound is administered at a dose of about 8 mg.

In some embodiments, the oligomeric compound is administered about once per month. In other embodiments, the oligomeric compound is administered more frequently than about once per month. In other embodiments, the oligomeric compound is administered about once every two, three, four, five, or six months, or once per year.

In some embodiments, administration of the oligomeric compound results in a decrease in the frequency of seizures experienced by the subject, as compared to the frequency of seizures experienced by the subject prior to administration of the oligomeric compound.

In some embodiments, administration of the oligomeric compound results in a reduction in the average number of daily seizures experienced by the subject in a 28-day period, as compared to the average number of daily seizures experienced by the subject in a 28-day period prior to administration of the oligomeric compound.

In some embodiments, administration of the oligomeric compound results in a reduction in the number of seizures experienced by the subject in a 28-day period by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80% or at least about 85%, as compared to the number of seizures experienced by the subject prior to administration of the oligomeric compound.

In some embodiments, administration of the oligomeric compound results in an increase in the number of seizure-free days experienced by the subject, as compared to the number of seizure-free days experienced by the subject prior to administration of the oligomeric compound.

In some embodiments, administration of the oligomeric compound results in an increase in the number of seizure-free days in a time period experienced by the subject, as compared to the number of seizure-free days experienced by the subject in the same time period prior to administration of the oligomeric compound

In some embodiments, administration of the oligomeric compound results in an increase in % of seizure-free days per 28-day period experienced by the subject, as compared to % of seizure-free days per 28-day period experienced by the subject prior to administration of the oligomeric compound.

In some embodiments, the seizures are selected from the group consisting of focal motor seizures, tonic seizures, generalized tonic-clonic seizures and myoclonic seizures.

In some embodiments, the subject is a human. In some embodiments, the subject is between 2 and 18 years old. In some embodiments, the subject is between 0 and 24 months old. In some embodiments, the subject is a newborn. In some embodiments, the newborn is a premature newborn. In some embodiments, the newborn has low birth weight with or without prematurity.

In some embodiments, the SCN2A-related disorder is SCN2A developmental and epileptic encephalopathy (SCN2A DEE). In some embodiments, the SCN2A-related disorder is early seizure onset epileptic encephalopathy (EE).

In some embodiments, the gain-of-function mutation in SCN2A gene is selected from the group consisting of L210Q, A263V, E430A, R1882Q, G879R, Q1479H, V423L, G1593R, K1502N, V1601L, G211D, S1780I, D343H and A1329D. In some embodiments, the gain-of-function mutation is A1329D.

In some embodiments, the oligomeric compound comprises a modified oligonucleotide represented by the following chemical notation: mCesmCeoAeomCeoGeoAeomCdsAdsTdsAdsTdsTdsTdsTdsTdsmCdsTeoAesmCesAe (SEQ ID NO: 3), wherein: A=an adenine nucleobase, mC=a 5-methyl cytosine nucleobase, G=a guanine nucleobase, T=a thymine nucleobase, e=a 2′-MOE sugar moiety, d=a 2′-β-D-deoxyribosyl sugar moiety, s=a phosphorothioate internucleoside linkage, and o=a phosphodiester internucleoside linkage.

In some embodiments, the oligomeric compound is a modified oligonucleotide represented by the following chemical structure:

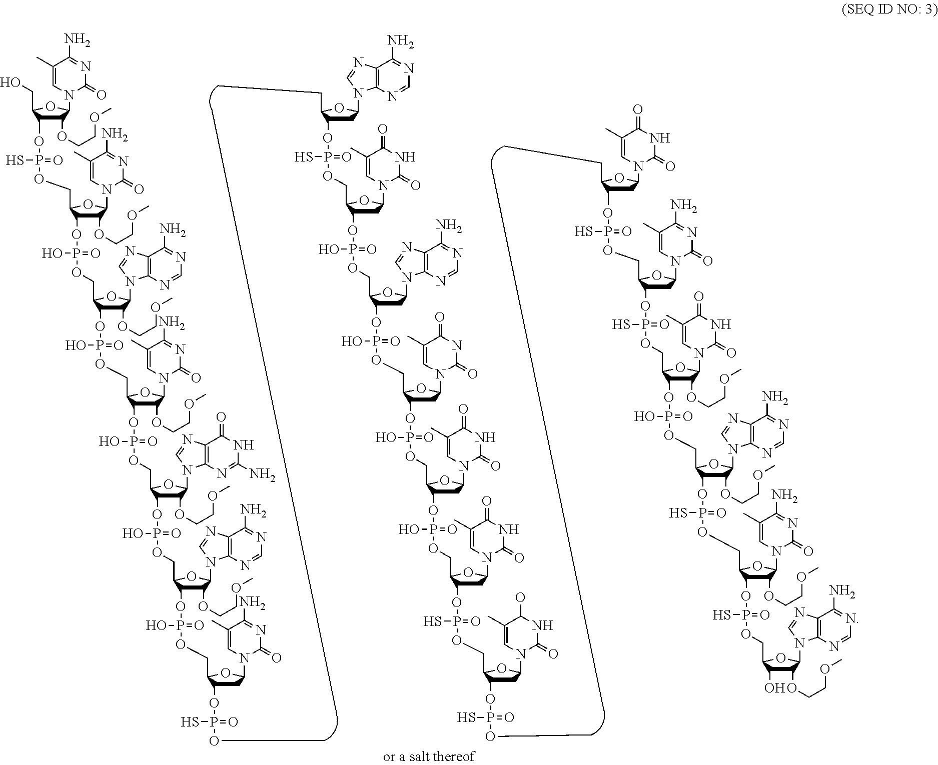

In some embodiments, the modified oligonucleotide is a sodium salt or a potassium salt. In some embodiments, the modified oligonucleotide is a sodium salt.

In some embodiments, the modified oligonucleotide represented by the following chemical structure:

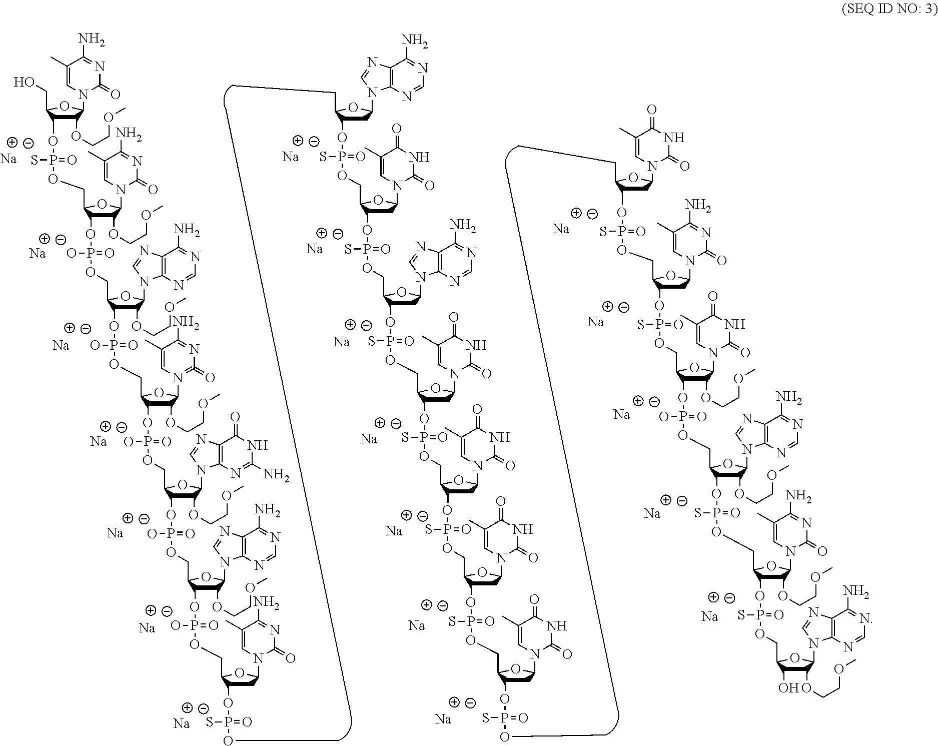

In some embodiments, the oligomeric compound is administered as a part of a pharmaceutical composition comprising the oligomeric compound and a pharmaceutically acceptable diluent or carrier. In some embodiments, the pharmaceutically acceptable diluent is artificial CSF (aCSF) or phosphate-buffered saline (PBS). In some embodiments, the pharmaceutically acceptable diluent is aCSF.

In some embodiments, the oligomeric compound is administered intrathecally.

### BRIEF DESCRIPTION OF THE DRAWINGS

FIG. 1 is a schematic illustrating dosing of the subjects in the clinical trial.

FIG. 2 is a bar graph showing median change in seizures vs. 28 day-baseline after 3 doses.

FIG. 3 is a bar graph showing % of seizure free day per dosing period, calculated as number of seizures over number of days between dose, per patient, after 3 doses.

FIG. 4, Panel A is a boxplot showing daily seizure frequency by dosing period (28 days) for subject 2001 after 3 doses.

FIG. 4, Panel B is a boxplot showing daily seizure frequency by dosing period (28 days) for subject 2002 after 3 doses.

FIG. 4, Panel C is a boxplot showing daily seizure frequency by dosing period (28 days) for subject 2003 after 3 doses.

FIG. 4, Panel D is a boxplot showing daily seizure frequency by dosing period (28 days) for subject 2004 after 3 doses.

FIG. 5, Panel A is a graph showing daily seizure frequency per dose period for subject 2001 after 3 doses.

FIG. 5, Panel B is a graph showing daily seizure frequency per dose period for subject 2002 after 3 doses.

FIG. 5, Panel C is a graph showing daily seizure frequency per dose period for subject 2003 after 3 doses.

FIG. 5, Panel D is a graph showing daily seizure frequency per dose period for subject 2004 after 3 doses.

FIG. 6, Panel A is a bar graph showing mean and median change from baseline in seizure frequency for four subjects. The results represent overall percentage reduction from baseline observed through four 28-day periods for 4 subjects.

FIG. 6, Panel B is a bar graph showing mean and median relative percentage change from baseline in seizure-free days for 4 subjects. The results represent overall relative percentage increase in proportion of seizure-free days for 4 subjects.

FIG. 7, Panel A shows representative aEEG traces in the subject showing a typical sawtooth pattern resembling EEG-status in week one.

FIG. 7, Panel B shows representative aEEG traces in the subject demonstrating seizure reduction after several loading doses of phenytoin (arrow) in week three.

FIG. 7, Panel C shows representative aEEG traces demonstrating that seizure reduction was not sustainable as status pattern reoccurred even when phenytoin levels were >40 g/mL. Seizures were subclinical or motor seizures.

FIG. 8, Panel A shows side and intracellular views of the 3D structure of Nav1.2 highlighting the A1329 residue in the intracellular linker between transmembrane segments S4 and S5 in domain III (S4-5_{DIII}). Also indicated are the four domains (DI-DIV), the inactivation gate and the β₂ subunit.

FIG. 8, Panel B shows zoomed-in views of S4-5_{DIII} region, before and after in silico mutagenesis (top, WT; bottom, A1329D). The D1329-F1489 interaction is likely to affect the binding of the IFM inactivation motif to its receptor pocket, resulting in delayed inactivation and persistent current.

FIG. 9, Panel A shows sodium current (I_{Na}) density-voltage relationships (inset voltage protocol). Representative I_{Na} traces are shown on the left.

FIG. 9, Panel B shows persistent inward I_{Na}-voltage relationships. Representative I_{Na} traces elicited by −10 mV depolarizations are shown on the left.

FIG. 9, Panel C shows voltage dependence of activation.

FIG. 9, Panel D shows voltage dependence of inactivation.

FIG. 9, Panel E shows dependence of the time course of I_{Na} inactivation on the membrane potential. Representative WT and A1329D INa traces elicited by −25 mV and −5 mV voltages are shown on the left.

FIG. 9, Panel F shows input-output relationships for WT and A1329D variant.

FIG. 10, Panel A shows patient clinical course including high-dose SCBs and introduction of Compound 1 dosing regimen. Associated reduction in seizure frequency is shown (bottom).

FIG. 10, Panel B is a bar graph showing the number of seizures per hour per medication level of phenytoin (PHT) and carbamazepine (CBZ). A total of seven Compound 1 intrathecal doses were administered (30.5 mg total dose).

FIG. 10, Panel C shows aEEG traces 1 day before the first administration of Compound 1 showing peak seizure frequency (status epilepticus).

FIG. 10, Panel D shows aEEG traces 1 week after first administration of Compound 1.

FIG. 10, Panel E shows aEEG traces 7 weeks after first administration of Compound 1.

FIG. 10, Panel F shows aEEG traces 10 weeks after first administration of Compound 1.

### DETAILED DESCRIPTION OF THE INVENTION

### Definitions

As used herein, the terms “about” and “approximately” refer to a value that is within 10% above or below the value being described.

As used herein, the term “SCN2A” refers to Naᵥ1.2, the alpha subunit of a voltage-gated sodium channel that is expressed in the brain, having an amino acid sequence from any vertebrate or mammalian source, including, but not limited to, human, bovine, chicken, rodent, mouse, rat, porcine, ovine, primate, monkey, and guinea pig, unless specified otherwise. SCN2A can, in some embodiments, include one or more mutations that lead to neurological diseases such as epileptic encephalitis. SCN2A is encoded by the SCN2A gene. In certain embodiments, SCN2A is a human SCN2A represented by SEQ ID NO: 1 as described herein.

Example accession numbers for human SCN2A mRNA transcript and human SCN2A protein are detailed below:

SCN2A mRNA transcript: NM_001040142.2 (SEQ ID NO: 4;
aacagacattgggtaccatcgaatgactgtcagaacagaaagctaaggcaaaggagggaggatgctgtggtcatcctttcttgtttttttcttct
ttaatgaggatagagcacatgtgagattttactttctactccagtaaaaattctgaagaattgcattggagactgttatattcaacacatacgtgga
ttctgtgttatgatttacatttttctttatttcagcactttcttatgcaaggagctaaacagtgattaaaggagcaggatgaaaagatggcacagtca
gtgctggtaccgccaggacctgacagcttccgcttctttaccagggaatcccttgctgctattgaacaacgcattgcagaagagaaagctaa
gagacccaaacaggaacgcaaggatgaggatgatgaaaatggcccaaagccaaacagtgacttggaagcaggaaaatctcttccatttatt
tatggagacattcctccagagatggtgtcagtgcccctggaggatctggacccctactatatcaataagaaaacgtttatagtattgaataaag
ggaaagcaatctctcgattcagtgccacccctgccctttacattttaactcccttcaaccctattagaaaattagctattaagattttggtacattctt
tattcaatatgctcattatgtgcacgattcttaccaactgtgtatttatgaccatgagtaaccctccagactggacaaagaatgtggagtatacctt
tacaggaatttatacttttgaatcacttattaaaatacttgcaaggggcttttgtttagaagatttcacatttttacgggatccatggaattggttgga
tttcacagtcattacttttgcatatgtgacagagtttgtggacctgggcaatgtctcagcgttgagaacattcagagttctccgagcattgaaaac
aatttcagtcattccaggcctgaagaccattgtgggggccctgatccagtcagtgaagaagctttctgatgtcatgatcttgactgtgttctgtct
aagcgtgtttgcgctaataggattgcagttgttcatgggcaacctacgaaataaatgtttgcaatggcctccagataattcttcctttgaaataaa
tatcacttccttctttaacaattcattggatgggaatggtactactttcaataggacagtgagcatatttaactgggatgaatatattgaggataaa
agtcacttttattttttagaggggcaaaatgatgctctgctttgtggcaacagctcagatgcaggccagtgtcctgaaggatacatctgtgtgaa
ggctggtagaaaccccaactatggctacacgagctttgacacctttagttgggcctttttgtccttatttcgtctcatgactcaagacttctggga
aaacctttatcaactgacactacgtgctgctgggaaaacgtacatgatattttttgtgctggtcattttcttgggctcattctatctaataaatttgat
cttggctgtggtggccatggcctatgaggaacagaatcaggccacattggaagaggctgaacagaaggaagctgaatttcagcagatgct
cgaacagttgaaaaagcaacaagaagaagctcaggcggcagctgcagccgcatctgctgaatcaagagacttcagtggtgctggtggga
taggagttttttcagagagttcttcagtagcatctaagttgagctccaaaagtgaaaaagagctgaaaaacagaagaaagaaaaagaaacag
aaagaacagtctggagaagaagagaaaaatgacagagtccgaaaatcggaatctgaagacagcataagaagaaaaggtttccgtttttcct
tggaaggaagtaggctgacatatgaaaagagattttcttctccacaccagtccttactgagcatccgtggctcccttttctctccaagacgcaa
cagtagggcgagccttttcagcttcagaggtcgagcaaaggacattggctctgagaatgactttgctgatgatgagcacagcacctttgagg
acaatgacagccgaagagactctctgttcgtgccgcacagacatggagaacggcgccacagcaatgtcagccaggccagccgtgcctcc
agggtgctccccatcctgcccatgaatgggaagatgcatagcgctgtggactgcaatggtgtggtctccctggtcgggggcccttctaccct
cacatctgctgggcagctcctaccagagggcacaactactgaaacagaaataagaaagagacggtccagttcttatcatgtttccatggattt
attggaagatcctacatcaaggcaaagagcaatgagtatagccagtattttgaccaacaccatggaagaacttgaagaatccagacagaaat
gcccaccatgctggtataaatttgctaatatgtgtttgatttgggactgttgtaaaccatggttaaaggtgaaacaccttgtcaacctggttgtaat
ggacccatttgttgacctggccatcaccatctgcattgtcttaaatacactcttcatggctatggagcactatcccatgacggagcagttcagca
gtgtactgtctgttggaaacctggtcttcacagggatcttcacagcagaaatgtttctcaagataattgccatggatccatattattactttcaaga
aggctggaatatttttgatggttttattgtgagccttagtttaatggaacttggtttggcaaatgtggaaggattgtcagttctccgatcattccggc
tgctccgagttttcaagttggcaaaatcttggccaactctaaatatgctaattaagatcattggcaattctgtgggggctctaggaaacctcacct
tggtattggccatcatcgtcttcatttttgctgtggtcggcatgcagctctttggtaagagctacaaagaatgtgtctgcaagatttccaatgattg
tgaactcccacgctggcacatgcatgactttttccactccttcctgatcgtgttccgcgtgctgtgtggagagtggatagagaccatgtgggac
tgtatggaggtcgctggccaaaccatgtgccttactgtcttcatgatggtcatggtgattggaaatctagtggttctgaacctcttcttggccttg
cttttgagttccttcagttctgacaatcttgctgccactgatgatgataacgaaatgaataatctccagattgctgtgggaaggatgcagaaagg
aatcgattttgttaaaagaaaaatacgtgaatttattcagaaagcctttgttaggaagcagaaagctttagatgaaattaaaccgcttgaagatct
aaataataaaaaagacagctgtatttccaaccataccaccatagaaataggcaaagacctcaattatctcaaagacggaaatggaactacta
gtggcataggcagcagtgtagaaaaatatgtcgtggatgaaagtgattacatgtcatttataaacaaccctagcctcactgtgacagtaccaat
tgctgttggagaatctgactttgaaaatttaaatactgaagaattcagcagcgagtcagatatggaggaaagcaaagagaagctaaatgcaa
ctagttcatctgaaggcagcacggttgatattggagctcccgccgagggagaacagcctgaggttgaacctgaggaatcccttgaacctga
agcctgttttacagaagactgtgtacggaagttcaagtgttgtcagataagcatagaagaaggcaaagggaaactctggtggaatttgagga
aaacatgctataagatagtggagcacaattggttcgaaaccttcattgtcttcatgattctgctgagcagtggggctctggcctttgaagatatat
acattgagcagcgaaaaaccattaagaccatgttagaatatgctgacaaggttttcacttacatattcattctggaaatgctgctaaagtgggtt
gcatatggttttcaagtgtattttaccaatgcctggtgctggctagacttcctgattgttgatgtctcactggttagcttaactgcaaatgccttggg
ttactcagaacttggtgccatcaaatccctcagaacactaagagctctgaggccactgagagctttgtcccggtttgaaggaatgagggttgtt
gtaaatgctcttttaggagccattccatctatcatgaatgtacttctggtttgtctgatcttttggctaatattcagtatcatgggagtgaatctctttg
ctggcaagttttaccattgtattaattacaccactggagagatgtttgatgtaagcgtggtcaacaactacagtgagtgcaaagctctcattgag
agcaatcaaactgccaggtggaaaaatgtgaaagtaaactttgataacgtaggacttggatatctgtctctacttcaagtagccacgtttaagg
gatggatggatattatgtatgcagctgttgattcacgaaatgtagaattacaacccaagtatgaagacaacctgtacatgtatctttattttgtcat
ctttattatttttggttcattctttaccttgaatcttttcattggtgtcatcatagataacttcaaccaacagaaaaagaagtttggaggtcaagacatt
tttatgacagaagaacagaagaaatactacaatgcaatgaaaaaactgggttcaaagaaaccacaaaaacccatacctcgacctgctaaca
aattccaaggaatggtctttgattttgtaaccaaacaagtctttgatatcagcatcatgatcctcatctgccttaacatggtcaccatgatggtgga
aaccgatgaccagagtcaagaaatgacaaacattctgtactggattaatctggtgtttattgttctgttcactggagaatgtgtgctgaaactgat
ctctcttcgttactactatttcactattggatggaatatttttgattttgtggtggtcattctctccattgtaggaatgtttctggctgaactgatagaa
aagtattttgtgtcccctaccctgttccgagtgatccgtcttgccaggattggccgaatcctacgtctgatcaaaggagcaaaggggatccgca
cgctgctctttgctttgatgatgtcccttcctgcgttgtttaacatcggcctccttcttttcctggtcatgttcatctacgccatctttgggatgtccaa
ttttgcctatgttaagagggaagttgggatcgatgacatgttcaactttgagacctttggcaacagcatgatctgcctgttccaaattacaacctc
tgctggctgggatggattgctagcacctattcttaatagtggacctccagactgtgaccctgacaaagatcaccctggaagctcagttaaagg
agactgtgggaacccatctgttgggattttcttttttgtcagttacatcatcatatccttcctggttgtggtgaacatgtacatcgcggtcatcctgg
agaacttcagtgttgctactgaagaaagtgcagagcctctgagtgaggatgactttgagatgttctatgaggtttgggagaagtttgatcccga
tgcgacccagtttatagagtttgccaaactttctgattttgcagatgccctggatcctcctcttctcatagcaaaacccaacaaagtccagctcat
tgccatggatctgcccatggtgagtggtgaccggatccactgtcttgacatcttatttgcttttacaaagcgtgttttgggtgagagtggagaga
tggatgcccttcgaatacagatggaagagcgattcatggcatcaaacccctccaaagtctcttatgagcccattacgaccacgttgaaacgc
aaacaagaggaggtgtctgctattattatccagagggcttacagacgctacctcttgaagcaaaaagttaaaaaggtatcaagtatatacaag
aaagacaaaggcaaagaatgtgatggaacacccatcaaagaagatactctcattgataaactgaatgagaattcaactccagagaaaaccg
atatgacgccttccaccacgtctccaccctcgtatgatagtgtgaccaaaccagaaaaagaaaaatttgaaaaagacaaatcagaaaagga
agacaaagggaaagatatcagggaaagtaaaaagtaaaaagaaaccaagaattttccattttgtgatcaattgtttacagcccgtgatggtga
tgtgtttgtgtcaacaggactcccacaggaggtctatgccaaactgactgtttttacaaatgtatacttaaggtcagtgcctataacaagacaga
gacctctggtcagcaaactggaactcagtaaactggagaaatagtatcgatgggaggtttctattttcacaaccagctgacactgctgaagag
cagaggcgtaatggctactcagacgataggaaccaatttaaaggggggagggaagttaaatttttatgtaaattcaacatgtgacacttgata
atagtaattgtcaccagtgtttatgttttaactgccacacctgccatatttttacaaaacgtgtgctgtgaatttatcacttttctttttaattcacag
gttgtttactattatatgtgactatttttgtaaatgggtttgtgtttggggagagggattaaagggagggaattctacatttctctattgtattgtata
actggatatattttaaatggaggcatgctgcaattctcattcacacataaaaaaatcacatcacaaaagggaagagtttacttcttgtttcaggatgtt
tttagatttttgaggtgcttaaatagctattcgtatttttaaggtgtctcatccagaaaaaatttaatgtgcctgtaaatgttccatagaatcacaagc
attaaagagttgttttatttttacataacccattaaatgtacatgtatatatgtatatatgtatatgtgcgtgtatatacatatatatgtatacacaca
tgcacacacagagatatacacataccattacattgtcattcacagtcccagcagcatgactatcacatttttgataagtgtcctttggcataaaataaa
aatatcctatcagtcctttctaagaagcctgaattgaccaaaaaacatccccaccaccactttataaagttgattctgctttatcctgcagtattgttta
gccatcttctgctcttggtaaggttgacatagtatatgtcaatttaaaaaataaaagtctgctttgtaaatagtaattttacccagtggtgcatgtttg
agcaaacaaaaatgatgatttaagcacactacttattgcatcaaatatgtaccacagtaagtatagtttgcaagctttcaacaggtaatatgatgt
aattggttccattatagtttgaagctgtcactgctgcatgtttatcttgcctatgctgctgtatcttattccttccactgttcagaagtctaatatggga
agccatatatcagtggtaaagtgaagcaaattgttctaccaagacctcattcttcatgtcattaagcaataggttgcagcaaacaaggaagagc
ttcttgctttttattcttccaaccttaattgaacactcaatgatgaaaagcccgactgtacaaacatgttgcaagctgcttaaatctgtttaaaatata
tggttagagttttctaagaaaatataaatactgtaaaaagttcattttattttatttttcagccttttgtacgtaaaatgagaaattaaaagtatcttca
ggtggatgtcacagtcactattgttagtttctgttcctagcacttttaaattgaagcacttcacaaaataagaagcaaggactaggatgcagtgtag
gtttctgcttttttattagtactgtaaacttgcacacatttcaatgtgaaacaaatctcaaactgagttcaatgtttatttgctttcaatagtaatgcct
tatcattgaaagaggcttaaagaaaaaaaaaatcagctgatactcttggcattgcttgaatccaatgtttccacctagtctttttattcagtaatcatc
agtcttttccaatgtttgtttacacagatagatcttattgacccatatggcactagaactgtatcagatataatatgggatcccagctttttttcctctc
ccacaaaaccaggtagtgaagttatattaccagttacagcaaaatactttgtgtttcacaagcaacaataaatgtagattctttatactgaagcta
ttgacttgtagtgtgttggtgaaatgcatgcaggaaaatgctgttaccataaagaacggtaaaccacattacaatcaagccaaaagaataaag
gtttcgcttttgtttttgtatttaattgttgtctttgtttctatctttgaaatgccatttaaaggtagatttctatcatgtaaaaataatctatctgaa
aaacaaatgtaaagaacacacattaattactataattcatctttcaattttttcatggaatggaagttaattaagaagagtgtattggataactacttt
aatattggccaaaaagctagatatggcatcaggtagactagtggaaagttacaaaaattaataaaaaattgactaaca),
NM_001040143.2 (SEQ ID NO: 5;
aacagacattgggtaccatcgaatgactgtcagaacagaaagctaaggcaaaggagggaggatgctgtggtcatcctttcttgtttttttcttct
ttaatgaggatagagcacatgtgagattttactttctactccagtaaaaattctgaagaattgcattggagactgttatattcaacacatacgtgga
ttctgtgttatgatttacatttttctttatttcaggggtttttctccctttgcttgacacttctctgtcctgacaccttgagaagaaggatgtgtttgc
ttacccttccgccatgattgtaaatttcctgaggccttcccagccatgcagcactcactttcttatgcaaggagctaaacagtgattaaaggagcagg
atgaaaagatggcacagtcagtgctggtaccgccaggacctgacagcttccgcttctttaccagggaatcccttgctgctattgaacaacgc
attgcagaagagaaagctaagagacccaaacaggaacgcaaggatgaggatgatgaaaatggcccaaagccaaacagtgacttggaag
caggaaaatctcttccatttatttatggagacattcctccagagatggtgtcagtgcccctggaggatctggacccctactatatcaataagaaa
acgtttatagtattgaataaagggaaagcaatctctcgattcagtgccacccctgccctttacattttaactcccttcaaccctattagaaaattag
ctattaagattttggtacattctttattcaatatgctcattatgtgcacgattcttaccaactgtgtatttatgaccatgagtaaccctccagactgga
caaagaatgtggagtatacctttacaggaatttatacttttgaatcacttattaaaatacttgcaaggggcttttgtttagaagatttcacatttttac
gggatccatggaattggttggatttcacagtcattacttttgcgtatgtaacagaatttgtaaacctaggcaatgtttcagctcttcgaactttcag
agtcttgagagctttgaaaactatttctgtaattccaggcctgaagaccattgtgggggccctgatccagtcagtgaagaagctttctgatgtca
tgatcttgactgtgttctgtctaagcgtgtttgcgctaataggattgcagttgttcatgggcaacctacgaaataaatgtttgcaatggcctccag
ataattcttcctttgaaataaatatcacttccttctttaacaattcattggatgggaatggtactactttcaataggacagtgagcatatttaactggg
atgaatatattgaggataaaagtcacttttattttttagaggggcaaaatgatgctctgctttgtggcaacagctcagatgcaggccagtgtcctg
aaggatacatctgtgtgaaggctggtagaaaccccaactatggctacacgagctttgacacctttagttgggcctttttgtccttatttcgtctcat
gactcaagacttctgggaaaacctttatcaactgacactacgtgctgctgggaaaacgtacatgatattttttgtgctggtcattttcttgggctca
ttctatctaataaatttgatcttggctgtggtggccatggcctatgaggaacagaatcaggccacattggaagaggctgaacagaaggaagct
gaatttcagcagatgctcgaacagttgaaaaagcaacaagaagaagctcaggcggcagctgcagccgcatctgctgaatcaagagacttc
agtggtgctggtgggataggagttttttcagagagttcttcagtagcatctaagttgagctccaaaagtgaaaaagagctgaaaaacagaaga
aagaaaaagaaacagaaagaacagtctggagaagaagagaaaaatgacagagtccgaaaatcggaatctgaagacagcataagaagaa
aaggtttccgtttttccttggaaggaagtaggctgacatatgaaaagagattttcttctccacaccagtccttactgagcatccgtggctccctttt
ctctccaagacgcaacagtagggcgagccttttcagcttcagaggtcgagcaaaggacattggctctgagaatgactttgctgatgatgagc
acagcacctttgaggacaatgacagccgaagagactctctgttcgtgccgcacagacatggagaacggcgccacagcaatgtcagccag
gccagccgtgcctccagggtgctccccatcctgcccatgaatgggaagatgcatagcgctgtggactgcaatggtgtggtctccctggtcg
ggggcccttctaccctcacatctgctgggcagctcctaccagagggcacaactactgaaacagaaataagaaagagacggtccagttctta
tcatgtttccatggatttattggaagatcctacatcaaggcaaagagcaatgagtatagccagtattttgaccaacaccatggaagaacttgaa
gaatccagacagaaatgcccaccatgctggtataaatttgctaatatgtgtttgatttgggactgttgtaaaccatggttaaaggtgaaacacctt
gtcaacctggttgtaatggacccatttgttgacctggccatcaccatctgcattgtcttaaatacactcttcatggctatggagcactatcccatg
acggagcagttcagcagtgtactgtctgttggaaacctggtcttcacagggatcttcacagcagaaatgtttctcaagataattgccatggatc
catattattactttcaagaaggctggaatatttttgatggttttattgtgagccttagtttaatggaacttggtttggcaaatgtggaaggattgtcag
ttctccgatcattccggctgctccgagttttcaagttggcaaaatcttggccaactctaaatatgctaattaagatcattggcaattctgtggggg
ctctaggaaacctcaccttggtattggccatcatcgtcttcatttttgctgtggtcggcatgcagctctttggtaagagctacaaagaatgtgtct
gcaagatttccaatgattgtgaactcccacgctggcacatgcatgactttttccactccttcctgatcgtgttccgcgtgctgtgtggagagtgg
atagagaccatgtgggactgtatggaggtcgctggccaaaccatgtgccttactgtcttcatgatggtcatggtgattggaaatctagtggttct
gaacctcttcttggccttgcttttgagttccttcagttctgacaatcttgctgccactgatgatgataacgaaatgaataatctccagattgctgtgg
gaaggatgcagaaaggaatcgattttgttaaaagaaaaatacgtgaatttattcagaaagcctttgttaggaagcagaaagctttagatgaaat
taaaccgcttgaagatctaaataataaaaaagacagctgtatttccaaccataccaccatagaaataggcaaagacctcaattatctcaaaga
cggaaatggaactactagtggcataggcagcagtgtagaaaaatatgtcgtggatgaaagtgattacatgtcatttataaacaaccctagcct
cactgtgacagtaccaattgctgttggagaatctgactttgaaaatttaaatactgaagaattcagcagcgagtcagatatggaggaaagcaa
agagaagctaaatgcaactagttcatctgaaggcagcacggttgatattggagctcccgccgagggagaacagcctgaggttgaacctga
ggaatcccttgaacctgaagcctgttttacagaagactgtgtacggaagttcaagtgttgtcagataagcatagaagaaggcaaagggaaac
tctggtggaatttgaggaaaacatgctataagatagtggagcacaattggttcgaaaccttcattgtcttcatgattctgctgagcagtggggct
ctggcctttgaagatatatacattgagcagcgaaaaaccattaagaccatgttagaatatgctgacaaggttttcacttacatattcattctggaa
atgctgctaaagtgggttgcatatggttttcaagtgtattttaccaatgcctggtgctggctagacttcctgattgttgatgtctcactggttagctt
aactgcaaatgccttgggttactcagaacttggtgccatcaaatccctcagaacactaagagctctgaggccactgagagctttgtcccggttt
gaaggaatgagggttgttgtaaatgctcttttaggagccattccatctatcatgaatgtacttctggtttgtctgatcttttggctaatattcagtatc
atgggagtgaatctctttgctggcaagttttaccattgtattaattacaccactggagagatgtttgatgtaagcgtggtcaacaactacagtgag
tgcaaagctctcattgagagcaatcaaactgccaggtggaaaaatgtgaaagtaaactttgataacgtaggacttggatatctgtctctacttca
agtagccacgtttaagggatggatggatattatgtatgcagctgttgattcacgaaatgtagaattacaacccaagtatgaagacaacctgtac
atgtatctttattttgtcatctttattatttttggttcattctttaccttgaatcttttcattggtgtcatcatagataacttcaaccaacagaaaaag
aagtttggaggtcaagacatttttatgacagaagaacagaagaaatactacaatgcaatgaaaaaactgggttcaaagaaaccacaaaaacccata
cctcgacctgctaacaaattccaaggaatggtctttgattttgtaaccaaacaagtctttgatatcagcatcatgatcctcatctgccttaacatgg
tcaccatgatggtggaaaccgatgaccagagtcaagaaatgacaaacattctgtactggattaatctggtgtttattgttctgttcactggagaa
tgtgtgctgaaactgatctctcttcgttactactatttcactattggatggaatatttttgattttgtggtggtcattctctccattgtaggaatgttt
ctggctgaactgatagaaaagtattttgtgtcccctaccctgttccgagtgatccgtcttgccaggattggccgaatcctacgtctgatcaaaggag
caaaggggatccgcacgctgctctttgctttgatgatgtcccttcctgcgttgtttaacatcggcctccttcttttcctggtcatgttcatctacgcc
atctttgggatgtccaattttgcctatgttaagagggaagttgggatcgatgacatgttcaactttgagacctttggcaacagcatgatctgcctg
ttccaaattacaacctctgctggctgggatggattgctagcacctattcttaatagtggacctccagactgtgaccctgacaaagatcaccctg
gaagctcagttaaaggagactgtgggaacccatctgttgggattttcttttttgtcagttacatcatcatatccttcctggttgtggtgaacatgtac
atcgcggtcatcctggagaacttcagtgttgctactgaagaaagtgcagagcctctgagtgaggatgactttgagatgttctatgaggtttggg
agaagtttgatcccgatgcgacccagtttatagagtttgccaaactttctgattttgcagatgccctggatcctcctcttctcatagcaaaaccca
acaaagtccagctcattgccatggatctgcccatggtgagtggtgaccggatccactgtcttgacatcttatttgcttttacaaagcgtgttttgg
gtgagagtggagagatggatgcccttcgaatacagatggaagagcgattcatggcatcaaacccctccaaagtctcttatgagcccattacg
accacgttgaaacgcaaacaagaggaggtgtctgctattattatccagagggcttacagacgctacctcttgaagcaaaaagttaaaaaggt
atcaagtatatacaagaaagacaaaggcaaagaatgtgatggaacacccatcaaagaagatactctcattgataaactgaatgagaattcaa
ctccagagaaaaccgatatgacgccttccaccacgtctccaccctcgtatgatagtgtgaccaaaccagaaaaagaaaaatttgaaaaaga
caaatcagaaaaggaagacaaagggaaagatatcagggaaagtaaaaagtaaaaagaaaccaagaattttccattttgtgatcaattgtttac
agcccgtgatggtgatgtgtttgtgtcaacaggactcccacaggaggtctatgccaaactgactgtttttacaaatgtatacttaaggtcagtgc
ctataacaagacagagacctctggtcagcaaactggaactcagtaaactggagaaatagtatcgatgggaggtttctattttcacaaccagct
gacactgctgaagagcagaggcgtaatggctactcagacgataggaaccaatttaaaggggggagggaagttaaatttttatgtaaattcaa
catgtgacacttgataatagtaattgtcaccagtgtttatgttttaactgccacacctgccatatttttacaaaacgtgtgctgtgaatttatcacttt
tctttttaattcacaggttgtttactattatatgtgactatttttgtaaatgggtttgtgtttggggagagggattaaagggagggaattctacatttc
tctattgtattgtataactggatatattttaaatggaggcatgctgcaattctcattcacacataaaaaaatcacatcacaaaagggaagagtttactt
cttgtttcaggatgtttttagatttttgaggtgcttaaatagctattcgtatttttaaggtgtctcatccagaaaaaatttaatgtgcctgtaaatgtt
ccatagaatcacaagcattaaagagttgttttatttttacataacccattaaatgtacatgtatatatgtatatatgtatatgtgcgtgtatatacata
tatatgtatacacacatgcacacacagagatatacacataccattacattgtcattcacagtcccagcagcatgactatcacatttttgataagtgtcc
tttggcataaaataaaaatatcctatcagtcctttctaagaagcctgaattgaccaaaaaacatccccaccaccactttataaagttgattctgcttt
atcctgcagtattgtttagccatcttctgctcttggtaaggttgacatagtatatgtcaatttaaaaaataaaagtctgctttgtaaatagtaatttta
cccagtggtgcatgtttgagcaaacaaaaatgatgatttaagcacactacttattgcatcaaatatgtaccacagtaagtatagtttgcaagctttc
aacaggtaatatgatgtaattggttccattatagtttgaagctgtcactgctgcatgtttatcttgcctatgctgctgtatcttattccttccactgtt
cagaagtctaatatgggaagccatatatcagtggtaaagtgaagcaaattgttctaccaagacctcattcttcatgtcattaagcaataggttgca
gcaaacaaggaagagcttcttgctttttattcttccaaccttaattgaacactcaatgatgaaaagcccgactgtacaaacatgttgcaagctgc
ttaaatctgtttaaaatatatggttagagttttctaagaaaatataaatactgtaaaaagttcattttattttatttttcagccttttgtacgtaaaa
tgagaaattaaaagtatcttcaggtggatgtcacagtcactattgttagtttctgttcctagcacttttaaattgaagcacttcacaaaataagaagca
aggactaggatgcagtgtaggtttctgcttttttattagtactgtaaacttgcacacatttcaatgtgaaacaaatctcaaactgagttcaatgtttat
ttgctttcaatagtaatgccttatcattgaaagaggcttaaagaaaaaaaaaatcagctgatactcttggcattgcttgaatccaatgtttccaccta
gtctttttattcagtaatcatcagtcttttccaatgtttgtttacacagatagatcttattgacccatatggcactagaactgtatcagatataatatg
ggatcccagctttttttcctctcccacaaaaccaggtagtgaagttatattaccagttacagcaaaatactttgtgtttcacaagcaacaataaatgt
agattctttatactgaagctattgacttgtagtgtgttggtgaaatgcatgcaggaaaatgctgttaccataaagaacggtaaaccacattacaat
caagccaaaagaataaaggtttcgcttttgtttttgtatttaattgttgtctttgtttctatctttgaaatgccatttaaaggtagatttctatcatgt
aaaaataatctatctgaaaaacaaatgtaaagaacacacattaattactataattcatctttcaattttttcatggaatggaagttaattaagaagagt
gtattggataactactttaatattggccaaaaagctagatatggcatcaggtagactagtggaaagttacaaaaattaataaaaaattgactaaca),
NM_001371246.1 (SEQ ID NO: 6;
aacagacattgggtaccatcgaatgactgtcagaacagaaagctaaggcaaaggagggaggatgctgtggtcatcctttcttgtttttttcttct
ttaatgaggatagagcacatgtgagattttactttctactccagtaaaaattctgaagaattgcattggagactgttatattcaacacatacgtgga
ttctgtgttatgatttacatttttctttatttcagcactttcttatgcaaggagctaaacagtgattaaaggagcaggatgaaaagatggcacagtca
gtgctggtaccgccaggacctgacagcttccgcttctttaccagggaatcccttgctgctattgaacaacgcattgcagaagagaaagctaa
gagacccaaacaggaacgcaaggatgaggatgatgaaaatggcccaaagccaaacagtgacttggaagcaggaaaatctcttccatttatt
tatggagacattcctccagagatggtgtcagtgcccctggaggatctggacccctactatatcaataagaaaacgtttatagtattgaataaag
ggaaagcaatctctcgattcagtgccacccctgccctttacattttaactcccttcaaccctattagaaaattagctattaagattttggtacattctt
tattcaatatgctcattatgtgcacgattcttaccaactgtgtatttatgaccatgagtaaccctccagactggacaaagaatgtggagtatacctt
tacaggaatttatacttttgaatcacttattaaaatacttgcaaggggcttttgtttagaagatttcacatttttacgggatccatggaattggttgga
tttcacagtcattacttttgcgtatgtaacagaatttgtaaacctaggcaatgtttcagctcttcgaactttcagagtcttgagagctttgaaaactat
ttctgtaattccaggcctgaagaccattgtgggggccctgatccagtcagtgaagaagctttctgatgtcatgatcttgactgtgttctgtctaag
cgtgtttgcgctaataggattgcagttgttcatgggcaacctacgaaataaatgtttgcaatggcctccagataattcttcctttgaaataaatatc
acttccttctttaacaattcattggatgggaatggtactactttcaataggacagtgagcatatttaactgggatgaatatattgaggataaaagtc
acttttattttttagaggggcaaaatgatgctctgctttgtggcaacagctcagatgcaggccagtgtcctgaaggatacatctgtgtgaaggct
ggtagaaaccccaactatggctacacgagctttgacacctttagttgggcctttttgtccttatttcgtctcatgactcaagacttctgggaaaac
ctttatcaactgacactacgtgctgctgggaaaacgtacatgatattttttgtgctggtcattttcttgggctcattctatctaataaatttgatcttg
gctgtggtggccatggcctatgaggaacagaatcaggccacattggaagaggctgaacagaaggaagctgaatttcagcagatgctcgaac
agttgaaaaagcaacaagaagaagctcaggcggcagctgcagccgcatctgctgaatcaagagacttcagtggtgctggtgggatagga
gttttttcagagagttcttcagtagcatctaagttgagctccaaaagtgaaaaagagctgaaaaacagaagaaagaaaaagaaacagaaaga
acagtctggagaagaagagaaaaatgacagagtccgaaaatcggaatctgaagacagcataagaagaaaaggtttccgtttttccttggaa
ggaagtaggctgacatatgaaaagagattttcttctccacaccagtccttactgagcatccgtggctcccttttctctccaagacgcaacagta
gggcgagccttttcagcttcagaggtcgagcaaaggacattggctctgagaatgactttgctgatgatgagcacagcacctttgaggacaat
gacagccgaagagactctctgttcgtgccgcacagacatggagaacggcgccacagcaatgtcagccaggccagccgtgcctccaggg
tgctccccatcctgcccatgaatgggaagatgcatagcgctgtggactgcaatggtgtggtctccctggtcgggggcccttctaccctcacat
ctgctgggcagctcctaccagagggcacaactactgaaacagaaataagaaagagacggtccagttcttatcatgtttccatggatttattgg
aagatcctacatcaaggcaaagagcaatgagtatagccagtattttgaccaacaccatggaagaacttgaagaatccagacagaaatgccc
accatgctggtataaatttgctaatatgtgtttgatttgggactgttgtaaaccatggttaaaggtgaaacaccttgtcaacctggttgtaatggac
ccatttgttgacctggccatcaccatctgcattgtcttaaatacactcttcatggctatggagcactatcccatgacggagcagttcagcagtgt
actgtctgttggaaacctggtcttcacagggatcttcacagcagaaatgtttctcaagataattgccatggatccatattattactttcaagaagg
ctggaatatttttgatggttttattgtgagccttagtttaatggaacttggtttggcaaatgtggaaggattgtcagttctccgatcattccggctgct
ccgagttttcaagttggcaaaatcttggccaactctaaatatgctaattaagatcattggcaattctgtgggggctctaggaaacctcaccttggt
attggccatcatcgtcttcatttttgctgtggtcggcatgcagctctttggtaagagctacaaagaatgtgtctgcaagatttccaatgattgtgaa
ctcccacgctggcacatgcatgactttttccactccttcctgatcgtgttccgcgtgctgtgtggagagtggatagagaccatgtgggactgtat
ggaggtcgctggccaaaccatgtgccttactgtcttcatgatggtcatggtgattggaaatctagtggttctgaacctcttcttggccttgcttttg
agttccttcagttctgacaatcttgctgccactgatgatgataacgaaatgaataatctccagattgctgtgggaaggatgcagaaaggaatcg
attttgttaaaagaaaaatacgtgaatttattcagaaagcctttgttaggaagcagaaagctttagatgaaattaaaccgcttgaagatctaaata
ataaaaaagacagctgtatttccaaccataccaccatagaaataggcaaagacctcaattatctcaaagacggaaatggaactactagtggc
ataggcagcagtgtagaaaaatatgtcgtggatgaaagtgattacatgtcatttataaacaaccctagcctcactgtgacagtaccaattgctgt
tggagaatctgactttgaaaatttaaatactgaagaattcagcagcgagtcagatatggaggaaagcaaagagaagctaaatgcaactagtt
catctgaaggcagcacggttgatattggagctcccgccgagggagaacagcctgaggttgaacctgaggaatcccttgaacctgaagcct
gttttacagaagactgtgtacggaagttcaagtgttgtcagataagcatagaagaaggcaaagggaaactctggtggaatttgaggaaaaca
tgctataagatagtggagcacaattggttcgaaaccttcattgtcttcatgattctgctgagcagtggggctctggcctttgaagatatatacatt
gagcagcgaaaaaccattaagaccatgttagaatatgctgacaaggttttcacttacatattcattctggaaatgctgctaaagtgggttgcata
tggttttcaagtgtattttaccaatgcctggtgctggctagacttcctgattgttgatgtctcactggttagcttaactgcaaatgccttgggttactc
agaacttggtgccatcaaatccctcagaacactaagagctctgaggccactgagagctttgtcccggtttgaaggaatgagggttgttgtaaa
tgctcttttaggagccattccatctatcatgaatgtacttctggtttgtctgatcttttggctaatattcagtatcatgggagtgaatctctttgctgg
caagttttaccattgtattaattacaccactggagagatgtttgatgtaagcgtggtcaacaactacagtgagtgcaaagctctcattgagagcaa
tcaaactgccaggtggaaaaatgtgaaagtaaactttgataacgtaggacttggatatctgtctctacttcaagtagccacgtttaagggatgg
atggatattatgtatgcagctgttgattcacgaaatgtagaattacaacccaagtatgaagacaacctgtacatgtatctttattttgtcatctttatt
atttttggttcattctttaccttgaatcttttcattggtgtcatcatagataacttcaaccaacagaaaaagaagtttggaggtcaagacatttttatg
acagaagaacagaagaaatactacaatgcaatgaaaaaactgggttcaaagaaaccacaaaaacccatacctcgacctgctaacaaattcca
aggaatggtctttgattttgtaaccaaacaagtctttgatatcagcatcatgatcctcatctgccttaacatggtcaccatgatggtggaaaccga
tgaccagagtcaagaaatgacaaacattctgtactggattaatctggtgtttattgttctgttcactggagaatgtgtgctgaaactgatctctctt
cgttactactatttcactattggatggaatatttttgattttgtggtggtcattctctccattgtaggaatgtttctggctgaactgatagaaaagtat
tttgtgtcccctaccctgttccgagtgatccgtcttgccaggattggccgaatcctacgtctgatcaaaggagcaaaggggatccgcacgctgc
tctttgctttgatgatgtcccttcctgcgttgtttaacatcggcctccttcttttcctggtcatgttcatctacgccatctttgggatgtccaattttg
cctatgttaagagggaagttgggatcgatgacatgttcaactttgagacctttggcaacagcatgatctgcctgttccaaattacaacctctgctggc
tgggatggattgctagcacctattcttaatagtggacctccagactgtgaccctgacaaagatcaccctggaagctcagttaaaggagactgt
gggaacccatctgttgggattttcttttttgtcagttacatcatcatatccttcctggttgtggtgaacatgtacatcgcggtcatcctggagaactt
cagtgttgctactgaagaaagtgcagagcctctgagtgaggatgactttgagatgttctatgaggtttgggagaagtttgatcccgatgcgac
ccagtttatagagtttgccaaactttctgattttgcagatgccctggatcctcctcttctcatagcaaaacccaacaaagtccagctcattgccat
ggatctgcccatggtgagtggtgaccggatccactgtcttgacatcttatttgcttttacaaagcgtgttttgggtgagagtgga gagatggatg
cccttcgaatacagatggaagagcgattcatggcatcaaacccctccaaagtctcttatgagcccattacgaccacgttgaaacgcaaacaa
gaggaggtgtctgctattattatccagagggcttacagacgctacctcttgaagcaaaaagttaaaaaggtatcaagtatatacaagaaagac
aaaggcaaagaatgtgatggaacacccatcaaagaagatactctcattgataaactgaatgagaattcaactccagagaaaaccgatatgac
gccttccaccacgtctccaccctcgtatgatagtgtgaccaaaccagaaaaagaaaaatttgaaaaagacaaatcagaaaaggaagacaaa
gggaaagatatcagggaaagtaaaaagtaaaaagaaaccaagaattttccattttgtgatcaattgtttacagcccgtgatggtgatgtgtttgt
gtcaacaggactcccacaggaggtctatgccaaactgactgtttttacaaatgtatacttaaggtcagtgcctataacaagacagagacctctg
gtcagcaaactggaactcagtaaactggagaaatagtatcgatgggaggtttctattttcacaaccagctgacactgctgaagagcagaggc
gtaatggctactcagacgataggaaccaatttaaaggggggagggaagttaaatttttatgtaaattcaacatgtgacacttgataatagtaatt
gtcaccagtgtttatgttttaactgccacacctgccatatttttacaaaacgtgtgctgtgaatttatcacttttctttttaattcacaggttgttt
actattatatgtgactatttttgtaaatgggtttgtgtttggggagagggattaaagggagggaattctacatttctctattgtattgtataactgga
tatattttaaatggaggcatgctgcaattctcattcacacataaaaaaatcacatcacaaaagggaagagtttacttcttgtttcaggatgtttttaga
tttttgaggtgcttaaatagctattcgtatttttaaggtgtctcatccagaaaaaatttaatgtgcctgtaaatgttccatagaatcacaagcattaa
agagttgttttatttttacataacccattaaatgtacatgtatatatgtatatatgtatatgtgcgtgtatatacatatatatgtatacacacatgca
cacacagagatatacacataccattacattgtcattcacagtcccagcagcatgactatcacatttttgataagtgtcctttggcataaaataaaaata
tcctatcagtcctttctaagaagcctgaattgaccaaaaaacatccccaccaccactttataaagttgattctgctttatcctgcagtattgtttagcc
atcttctgctcttggtaaggttgacatagtatatgtcaatttaaaaaataaaagtctgctttgtaaatagtaattttacccagtggtgcatgtttgagc
aaacaaaaatgatgatttaagcacactacttattgcatcaaatatgtaccacagtaagtatagtttgcaagctttcaacaggtaatatgatgtaattgg
ttccattatagtttgaagctgtcactgctgcatgtttatcttgcctatgctgctgtatcttattccttccactgttcagaagtctaatatgggaagcc
atatatcagtggtaaagtgaagcaaattgttctaccaagacctcattcttcatgtcattaagcaataggttgcagcaaacaaggaagagcttcttgctt
tttattcttccaaccttaattgaacactcaatgatgaaaagcccgactgtacaaacatgttgcaagctgcttaaatctgtttaaaatatatggttaga
gttttctaagaaaatataaatactgtaaaaagttcattttattttatttttcagccttttgtacgtaaaatgagaaattaaaagtatcttcaggtggat
gtcacagtcactattgttagtttctgttcctagcacttttaaattgaagcacttcacaaaataagaagcaaggactaggatgcagtgtaggtttctgct
tttttattagtactgtaaacttgcacacatttcaatgtgaaacaaatctcaaactgagttcaatgtttatttgctttcaatagtaatgccttatcattg
aaagaggcttaaagaaaaaaaaaatcagctgatactcttggcattgcttgaatccaatgtttccacctagtctttttattcagtaatcatcagtctttt
ccaatgtttgtttacacagatagatcttattgacccatatggcactagaactgtatcagatataatatgggatcccagctttttttcctctcccacaaa
accaggtagtgaagttatattaccagttacagcaaaatactttgtgtttcacaagcaacaataaatgtagattctttatactgaagctattgacttgt
agtgtgttggtgaaatgcatgcaggaaaatgctgttaccataaagaacggtaaaccacattacaatcaagccaaaagaataaaggtttcgctt
ttgtttttgtatttaattgttgtctttgtttctatctttgaaatgccatttaaaggtagatttctatcatgtaaaaataatctatctgaaaaacaaat
gtaaagaacacacattaattactataattcatctttcaattttttcatggaatggaagttaattaagaagagtgtattggataactactttaata
ttggccaaaaagctagatatggcatcaggtagactagtggaaagttacaaaaattaataaaaaattgactaaca),
NM_001371247.1 (SEQ ID NO: 7;
atagcagtaacacaattcacctctagtgtgaacatatcaggatggcatagaccagcactttcttatgcaaggagctaaacagtgattaaagga
gcaggatgaaaagatggcacagtcagtgctggtaccgccaggacctgacagcttccgcttctttaccagggaatcccttgctgctattgaac
aacgcattgcagaagagaaagctaagagacccaaacaggaacgcaaggatgaggatgatgaaaatggcccaaagccaaacagtgactt
ggaagcaggaaaatctcttccatttatttatggagacattcctccagagatggtgtcagtgcccctggaggatctggacccctactatatcaat
aagaaaacgtttatagtattgaataaagggaaagcaatctctcgattcagtgccacccctgccctttacattttaactcccttcaaccctattaga
aaattagctattaagattttggtacattctttattcaatatgctcattatgtgcacgattcttaccaactgtgtatttatgaccatgagtaaccctcca
gactggacaaagaatgtggagtatacctttacaggaatttatacttttgaatcacttattaaaatacttgcaaggggcttttgtttagaagatttcaca
tttttacgggatccatggaattggttggatttcacagtcattacttttgcatatgtgacagagtttgtggacctgggcaatgtctcagcgttgagaa
cattcagagttctccgagcattgaaaacaatttcagtcattccaggcctgaagaccattgtgggggccctgatccagtcagtgaagaagctttc
tgatgtcatgatcttgactgtgttctgtctaagcgtgtttgcgctaataggattgcagttgttcatgggcaacctacgaaataaatgtttgcaatgg
cctccagataattcttcctttgaaataaatatcacttccttctttaacaattcattggatgggaatggtactactttcaataggacagtgagcatattt
aactgggatgaatatattgaggataaaagtcacttttattttttagaggggcaaaatgatgctctgctttgtggcaacagctcagatgcaggcca
gtgtcctgaaggatacatctgtgtgaaggctggtagaaaccccaactatggctacacgagctttgacacctttagttgggcctttttgtccttatt
tcgtctcatgactcaagacttctgggaaaacctttatcaactgacactacgtgctgctgggaaaacgtacatgatattttttgtgctggtcattttct
tgggctcattctatctaataaatttgatcttggctgtggtggccatggcctatgaggaacagaatcaggccacattggaagaggctgaacaga
aggaagctgaatttcagcagatgctcgaacagttgaaaaagcaacaagaagaagctcaggcggcagctgcagccgcatctgctgaatcaa
gagacttcagtggtgctggtgggataggagttttttcagagagttcttcagtagcatctaagttgagctccaaaagtgaaaaagagctgaaaa
acagaagaaagaaaaagaaacagaaagaacagtctggagaagaagagaaaaatgacagagtccgaaaatcggaatctgaagacagcat
aagaagaaaaggtttccgtttttccttggaaggaagtaggctgacatatgaaaagagattttcttctccacaccagtccttactgagcatccgtg
gctcccttttctctccaagacgcaacagtagggcgagccttttcagcttcagaggtcgagcaaaggacattggctctgagaatgactttgctg
atgatgagcacagcacctttgaggacaatgacagccgaagagactctctgttcgtgccgcacagacatggagaacggcgccacagcaat
gtcagccaggccagccgtgcctccagggtgctccccatcctgcccatgaatgggaagatgcatagcgctgtggactgcaatggtgtggtct
ccctggtcgggggcccttctaccctcacatctgctgggcagctcctaccagagggcacaactactgaaacagaaataagaaagagacggt
ccagttcttatcatgtttccatggatttattggaagatcctacatcaaggcaaagagcaatgagtatagccagtattttgaccaacaccatggaa
gaacttgaagaatccagacagaaatgcccaccatgctggtataaatttgctaatatgtgtttgatttgggactgttgtaaaccatggttaaaggt
gaaacaccttgtcaacctggttgtaatggacccatttgttgacctggccatcaccatctgcattgtcttaaatacactcttcatggctatggagca
ctatcccatgacggagcagttcagcagtgtactgtctgttggaaacctggtcttcacagggatcttcacagcagaaatgtttctcaagataattg
ccatggatccatattattactttcaagaaggctggaatatttttgatggttttattgtgagccttagtttaatggaacttggtttggcaaatgtggaag
gattgtcagttctccgatcattccggctgctccgagttttcaagttggcaaaatcttggccaactctaaatatgctaattaagatcattggcaattc
tgtgggggctctaggaaacctcaccttggtattggccatcatcgtcttcatttttgctgtggtcggcatgcagctctttggtaagagctacaaag
aatgtgtctgcaagatttccaatgattgtgaactcccacgctggcacatgcatgactttttccactccttcctgatcgtgttccgcgtgctgtgtgg
agagtggatagagaccatgtgggactgtatggaggtcgctggccaaaccatgtgccttactgtcttcatgatggtcatggtgattggaaatct
agtggttctgaacctcttcttggccttgcttttgagttccttcagttctgacaatcttgctgccactgatgatgataacgaaatgaataatctccaga
ttgctgtgggaaggatgcagaaaggaatcgattttgttaaaagaaaaatacgtgaatttattcagaaagcctttgttaggaagcagaaagcttt
agatgaaattaaaccgcttgaagatctaaataataaaaaagacagctgtatttccaaccataccaccatagaaataggcaaagacctcaattat
ctcaaagacggaaatggaactactagtggcataggcagcagtgtagaaaaatatgtcgtggatgaaagtgattacatgtcatttataaacaac
cctagcctcactgtgacagtaccaattgctgttggagaatctgactttgaaaatttaaatactgaagaattcagcagcgagtcagatatggagg
aaagcaaagagaagctaaatgcaactagttcatctgaaggcagcacggttgatattggagctcccgccgagggagaacagcctgaggttg
aacctgaggaatcccttgaacctgaagcctgttttacagaagactgtgtacggaagttcaagtgttgtcagataagcatagaagaaggcaaa
gggaaactctggtggaatttgaggaaaacatgctataagatagtggagcacaattggttcgaaaccttcattgtcttcatgattctgctgagca
gtggggctctggcctttgaagatatatacattgagcagcgaaaaaccattaagaccatgttagaatatgctgacaaggttttcacttacatattc
attctggaaatgctgctaaagtgggttgcatatggttttcaagtgtattttaccaatgcctggtgctggctagacttcctgattgttgatgtctcact
ggttagcttaactgcaaatgccttgggttactcagaacttggtgccatcaaatccctcagaacactaagagctctgaggccactgagagctttg
tcccggtttgaaggaatgagggttgttgtaaatgctcttttaggagccattccatctatcatgaatgtacttctggtttgtctgatcttttggctaata
ttcagtatcatgggagtgaatctctttgctggcaagttttaccattgtattaattacaccactggagagatgtttgatgtaagcgtggtcaacaact
acagtgagtgcaaagctctcattgagagcaatcaaactgccaggtggaaaaatgtgaaagtaaactttgataacgtaggacttggatatctgt
ctctacttcaagtagccacgtttaagggatggatggatattatgtatgcagctgttgattcacgaaatgtagaattacaacccaagtatgaagac
aacctgtacatgtatctttattttgtcatctttattatttttggttcattctttaccttgaatcttttcattggtgtcatcatagataacttcaacca
acagaaaaagaagtttggaggtcaagacatttttatgacagaagaacagaagaaatactacaatgcaatgaaaaaactgggttcaaagaaaccacaa
aaacccatacctcgacctgctaacaaattccaaggaatggtctttgattttgtaaccaaacaagtctttgatatcagcatcatgatcctcatctgc
cttaacatggtcaccatgatggtggaaaccgatgaccagagtcaagaaatgacaaacattctgtactggattaatctggtgtttattgttctgttc
actggagaatgtgtgctgaaactgatctctcttcgttactactatttcactattggatggaatatttttgattttgtggtggtcattctctccattgt
aggaatgtttctggctgaactgatagaaaagtattttgtgtcccctaccctgttccgagtgatccgtcttgccaggattggccgaatcctacgtctga
tcaaaggagcaaaggggatccgcacgctgctctttgctttgatgatgtcccttcctgcgttgtttaacatcggcctccttcttttcctggtcatgtt
catctacgccatctttgggatgtccaattttgcctatgttaagagggaagttgggatcgatgacatgttcaactttgagacctttggcaacagcat
gatctgcctgttccaaattacaacctctgctggctgggatggattgctagcacctattcttaatagtggacctccagactgtgaccctgacaaa
gatcaccctggaagctcagttaaaggagactgtgggaacccatctgttgggattttcttttttgtcagttacatcatcatatccttcctggttgtggt
gaacatgtacatcgcggtcatcctggagaacttcagtgttgctactgaagaaagtgcagagcctctgagtgaggatgactttgagatgttctat
gaggtttgggagaagtttgatcccgatgcgacccagtttatagagtttgccaaactttctgattttgcagatgccctggatcctcctcttctcata
gcaaaacccaacaaagtccagctcattgccatggatctgcccatggtgagtggtgaccggatccactgtcttgacatcttatttgcttttacaaa
gcgtgttttgggtgagagtggagagatggatgcccttcgaatacagatggaagagcgattcatggcatcaaacccctccaaagtctcttatga
gcccattacgaccacgttgaaacgcaaacaagaggaggtgtctgctattattatccagagggcttacagacgctacctcttgaagcaaaaag
ttaaaaaggtatcaagtatatacaagaaagacaaaggcaaagaatgtgatggaacacccatcaaagaagatactctcattgataaactgaat
gagaattcaactccagagaaaaccgatatgacgccttccaccacgtctccaccctcgtatgatagtgtgaccaaaccagaaaaagaaaaatt
tgaaaaagacaaatcagaaaaggaagacaaagggaaagatatcagggaaagtaaaaagtaaaaagaaaccaagaattttccattttgtgat
caattgtttacagcccgtgatggtgatgtgtttgtgtcaacaggactcccacaggaggtctatgccaaactgactgtttttacaaatgtatactta
aggtcagtgcctataacaagacagagacctctggtcagcaaactggaactcagtaaactggagaaatagtatcgatgggaggtttctattttc
acaaccagctgacactgctgaagagcagaggcgtaatggctactcagacgataggaaccaatttaaaggggggagggaagttaaattttta
tgtaaattcaacatgtgacacttgataatagtaattgtcaccagtgtttatgttttaactgccacacctgccatatttttacaaaacgtgtgctgtga
atttatcacttttctttttaattcacaggttgtttactattatatgtgactatttttgtaaatgggtttgtgtttggggagagggattaaagggaggga
attctacatttctctattgtattgtataactggatatattttaaatggaggcatgctgcaattctcattcacacataaaaaaatcacatcacaaaaggg
aagagtttacttcttgtttcaggatgtttttagatttttgaggtgcttaaatagctattcgtatttttaaggtgtctcatccagaaaaaatttaatgtg
cctgtaaatgttccatagaatcacaagcattaaagagttgttttatttttacataacccattaaatgtacatgtatatatgtatatatgtatatgtgcg
tgtatatacatatatatgtatacacacatgcacacacagagatatacacataccattacattgtcattcacagtcccagcagcatgactatcacatttt
tgataagtgtcctttggcataaaataaaaatatcctatcagtcctttctaagaagcctgaattgaccaaaaaacatccccaccaccactttataaagt
tgattctgctttatcctgcagtattgtttagccatcttctgctcttggtaaggttgacatagtatatgtcaatttaaaaaataaaagtctgctttgtaa
atagtaattttacccagtggtgcatgtttgagcaaacaaaaatgatgatttaagcacactacttattgcatcaaatatgtaccacagtaagtatagttt
gcaagctttcaacaggtaatatgatgtaattggttccattatagtttgaagctgtcactgctgcatgtttatcttgcctatgctgctgtatcttattcc
ttccactgttcagaagtctaatatgggaagccatatatcagtggtaaagtgaagcaaattgttctaccaagacctcattcttcatgtcattaagcaa
taggttgcagcaaacaaggaagagcttcttgctttttattcttccaaccttaattgaacactcaatgatgaaaagcccgactgtacaaacatgttg
caagctgcttaaatctgtttaaaatatatggttagagttttctaagaaaatataaatactgtaaaaagttcattttattttatttttcagccttttgt
acgtaaaatgagaaattaaaagtatcttcaggtggatgtcacagtcactattgttagtttctgttcctagcacttttaaattgaagcacttcacaaaat
aagaagcaaggactaggatgcagtgtaggtttctgcttttttattagtactgtaaacttgcacacatttcaatgtgaaacaaatctcaaactgagttca
atgtttatttgctttcaatagtaatgccttatcattgaaagaggcttaaagaaaaaaaaaatcagctgatactcttggcattgcttgaatccaatgtt
tccacctagtctttttattcagtaatcatcagtcttttccaatgtttgtttacacagatagatcttattgacccatatggcactagaactgtatcagat
ataatatgggatcccagctttttttcctctcccacaaaaccaggtagtgaagttatattaccagttacagcaaaatactttgtgtttcacaagcaaca
ataaatgtagattctttatactgaagctattgacttgtagtgtgttggtgaaatgcatgcaggaaaatgctgttaccataaagaacggtaaaccac
attacaatcaagccaaaagaataaaggtttcgcttttgtttttgtatttaattgttgtctttgtttctatctttgaaatgccatttaaaggtagatttc
tatcatgtaaaaataatctatctgaaaaacaaatgtaaagaacacacattaattactataattcatctttcaattttttcatggaatggaagttaatt
aagaagagtgtattggataactactttaatattggccaaaaagctagatatggcatcaggtagactagtggaaagttacaaaaattaataaaaaattga
ctaaca),
and
NM_021007.3 (SEQ ID NO: 2;
aagcatgatggaattttagctgcagtcttcttggtgccagcttatcaatcccaaactctgggtgtaaaagattctacagggcactttcttatgcaa
ggagctaaacagtgattaaaggagcaggatgaaaagatggcacagtcagtgctggtaccgccaggacctgacagcttccgcttctttacca
gggaatcccttgctgctattgaacaacgcattgcagaagagaaagctaagagacccaaacaggaacgcaaggatgaggatgatgaaaat
ggcccaaagccaaacagtgacttggaagcaggaaaatctcttccatttatttatggagacattcctccagagatggtgtcagtgcccctggag
gatctggacccctactatatcaataagaaaacgtttatagtattgaataaagggaaagcaatctctcgattcagtgccacccctgccctttacatt
ttaactcccttcaaccctattagaaaattagctattaagattttggtacattctttattcaatatgctcattatgtgcacgattcttaccaactgtgta
tttatgaccatgagtaaccctccagactggacaaagaatgtggagtatacctttacaggaatttatacttttgaatcacttattaaaatacttgcaagg
ggcttttgtttagaagatttcacatttttacgggatccatggaattggttggatttcacagtcattacttttgcatatgtgacagagtttgtggacctg
ggcaatgtctcagcgttgagaacattcagagttctccgagcattgaaaacaatttcagtcattccaggcctgaagaccattgtgggggccctg
atccagtcagtgaagaagctttctgatgtcatgatcttgactgtgttctgtctaagcgtgtttgcgctaataggattgcagttgttcatgggcaacc
tacgaaataaatgtttgcaatggcctccagataattcttcctttgaaataaatatcacttccttctttaacaattcattggatgggaatggtactactt
tcaataggacagtgagcatatttaactgggatgaatatattgaggataaaagtcacttttattttttagaggggcaaaatgatgctctgctttgtgg
caacagctcagatgcaggccagtgtcctgaaggatacatctgtgtgaaggctggtagaaaccccaactatggctacacgagctttgacacct
ttagttgggcctttttgtccttatttcgtctcatgactcaagacttctgggaaaacctttatcaactgacactacgtgctgctgggaaaacgtacat
gatattttttgtgctggtcattttcttgggctcattctatctaataaatttgatcttggctgtggtggccatggcctatgaggaacagaatcaggcca
cattggaagaggctgaacagaaggaagctgaatttcagcagatgctcgaacagttgaaaaagcaacaagaagaagctcaggcggcagct
gcagccgcatctgctgaatcaagagacttcagtggtgctggtgggataggagttttttcagagagttcttcagtagcatctaagttgagctcca
aaagtgaaaaagagctgaaaaacagaagaaagaaaaagaaacagaaagaacagtctggagaagaagagaaaaatgacagagtccgaa
aatcggaatctgaagacagcataagaagaaaaggtttccgtttttccttggaaggaagtaggctgacatatgaaaagagattttcttctccaca
ccagtccttactgagcatccgtggctcccttttctctccaagacgcaacagtagggcgagccttttcagcttcagaggtcgagcaaaggacat
tggctctgagaatgactttgctgatgatgagcacagcacctttgaggacaatgacagccgaagagactctctgttcgtgccgcacagacatg
gagaacggcgccacagcaatgtcagccaggccagccgtgcctccagggtgctccccatcctgcccatgaatgggaagatgcatagcgct
gtggactgcaatggtgtggtctccctggtcgggggcccttctaccctcacatctgctgggcagctcctaccagagggcacaactactgaaac
agaaataagaaagagacggtccagttcttatcatgtttccatggatttattggaagatcctacatcaaggcaaagagcaatgagtatagccagt
attttgaccaacaccatggaagaacttgaagaatccagacagaaatgcccaccatgctggtataaatttgctaatatgtgtttgatttgggactg
ttgtaaaccatggttaaaggtgaaacaccttgtcaacctggttgtaatggacccatttgttgacctggccatcaccatctgcattgtcttaaatac
actcttcatggctatggagcactatcccatgacggagcagttcagcagtgtactgtctgttggaaacctggtcttcacagggatcttcacagca
gaaatgtttctcaagataattgccatggatccatattattactttcaagaaggctggaatatttttgatggttttattgtgagccttagtttaatggaa
cttggtttggcaaatgtggaaggattgtcagttctccgatcattccggctgctccgagttttcaagttggcaaaatcttggccaactctaaatatg
ctaattaagatcattggcaattctgtgggggctctaggaaacctcaccttggtattggccatcatcgtcttcatttttgctgtggtcggcatgcag
ctctttggtaagagctacaaagaatgtgtctgcaagatttccaatgattgtgaactcccacgctggcacatgcatgactttttccactccttcctg
atcgtgttccgcgtgctgtgtggagagtggatagagaccatgtgggactgtatggaggtcgctggccaaaccatgtgccttactgtcttcatg
atggtcatggtgattggaaatctagtggttctgaacctcttcttggccttgcttttgagttccttcagttctgacaatcttgctgccactgatgatgat
aacgaaatgaataatctccagattgctgtgggaaggatgcagaaaggaatcgattttgttaaaagaaaaatacgtgaatttattcagaaagcct
ttgttaggaagcagaaagctttagatgaaattaaaccgcttgaagatctaaataataaaaaagacagctgtatttccaaccataccaccataga
aataggcaaagacctcaattatctcaaagacggaaatggaactactagtggcataggcagcagtgtagaaaaatatgtcgtggatgaaagt
gattacatgtcatttataaacaaccctagcctcactgtgacagtaccaattgctgttggagaatctgactttgaaaatttaaatactgaagaattca
gcagcgagtcagatatggaggaaagcaaagagaagctaaatgcaactagttcatctgaaggcagcacggttgatattggagctcccgccg
agggagaacagcctgaggttgaacctgaggaatcccttgaacctgaagcctgttttacagaagactgtgtacggaagttcaagtgttgtcag
ataagcatagaagaaggcaaagggaaactctggtggaatttgaggaaaacatgctataagatagtggagcacaattggttcgaaaccttcat
tgtcttcatgattctgctgagcagtggggctctggcctttgaagatatatacattgagcagcgaaaaaccattaagaccatgttagaatatgctg
acaaggttttcacttacatattcattctggaaatgctgctaaagtgggttgcatatggttttcaagtgtattttaccaatgcctggtgctggctagac
ttcctgattgttgatgtctcactggttagcttaactgcaaatgccttgggttactcagaacttggtgccatcaaatccctcagaacactaagagct
ctgaggccactgagagctttgtcccggtttgaaggaatgagggttgttgtaaatgctcttttaggagccattccatctatcatgaatgtacttctg
gtttgtctgatcttttggctaatattcagtatcatgggagtgaatctctttgctggcaagttttaccattgtattaattacaccactggagagatgttt
gatgtaagcgtggtcaacaactacagtgagtgcaaagctctcattgagagcaatcaaactgccaggtggaaaaatgtgaaagtaaactttgat
aacgtaggacttggatatctgtctctacttcaagtagccacgtttaagggatggatggatattatgtatgcagctgttgattcacgaaatgtagaa
ttacaacccaagtatgaagacaacctgtacatgtatctttattttgtcatctttattatttttggttcattctttaccttgaatcttttcattggtgt
catcatagataacttcaaccaacagaaaaagaagtttggaggtcaagacatttttatgacagaagaacagaagaaatactacaatgcaatgaaaaaac
tgggttcaaagaaaccacaaaaacccatacctcgacctgctaacaaattccaaggaatggtctttgattttgtaaccaaacaagtctttgatatc
agcatcatgatcctcatctgccttaacatggtcaccatgatggtggaaaccgatgaccagagtcaagaaatgacaaacattctgtactggatta
atctggtgtttattgttctgttcactggagaatgtgtgctgaaactgatctctcttcgttactactatttcactattggatggaatatttttgatttt
gtggtggtcattctctccattgtaggaatgtttctggctgaactgatagaaaagtattttgtgtcccctaccctgttccgagtgatccgtcttgccag
gattggccgaatcctacgtctgatcaaaggagcaaaggggatccgcacgctgctctttgctttgatgatgtcccttcctgcgttgtttaacatcggcc
tccttcttttcctggtcatgttcatctacgccatctttgggatgtccaattttgcctatgttaagagggaagttgggatcgatgacatgttcaactttg
agacctttggcaacagcatgatctgcctgttccaaattacaacctctgctggctgggatggattgctagcacctattcttaatagtggacctcca
gactgtgaccctgacaaagatcaccctggaagctcagttaaaggagactgtgggaacccatctgttgggattttcttttttgtcagttacatcat
catatccttcctggttgtggtgaacatgtacatcgcggtcatcctggagaacttcagtgttgctactgaagaaagtgcagagcctctgagtgag
gatgactttgagatgttctatgaggtttgggagaagtttgatcccgatgcgacccagtttatagagtttgccaaactttctgattttgcagatgccc
tggatcctcctcttctcatagcaaaacccaacaaagtccagctcattgccatggatctgcccatggtgagtggtgaccggatccactgtcttga
catcttatttgcttttacaaagcgtgttttgggtgagagtggagagatggatgcccttcgaatacagatggaagagcgattcatggcatcaaac
ccctccaaagtctcttatgagcccattacgaccacgttgaaacgcaaacaagaggaggtgtctgctattattatccagagggcttacagacgc
tacctcttgaagcaaaaagttaaaaaggtatcaagtatatacaagaaagacaaaggcaaagaatgtgatggaacacccatcaaagaagata
ctctcattgataaactgaatgagaattcaactccagagaaaaccgatatgacgccttccaccacgtctccaccctcgtatgatagtgtgaccaa
accagaaaaagaaaaatttgaaaaagacaaatcagaaaaggaagacaaagggaaagatatcagggaaagtaaaaagtaaaaagaaacc
aagaattttccattttgtgatcaattgtttacagcccgtgatggtgatgtgtttgtgtcaacaggactcccacaggaggtctatgccaaactgact
gtttttacaaatgtatacttaaggtcagtgcctataacaagacagagacctctggtcagcaaactggaactcagtaaactggagaaatagtatc
gatgggaggtttctattttcacaaccagctgacactgctgaagagcagaggcgtaatggctactcagacgataggaaccaatttaaagggg
ggagggaagttaaatttttatgtaaattcaacatgtgacacttgataatagtaattgtcaccagtgtttatgttttaactgccacacctgccatatttt
tacaaaacgtgtgctgtgaatttatcacttttctttttaattcacaggttgtttactattatatgtgactatttttgtaaatgggtttgtgtttgggga
gagggattaaagggagggaattctacatttctctattgtattgtataactggatatattttaaatggaggcatgctgcaattctcattcacacataaaa
aaatcacatcacaaaagggaagagtttacttcttgtttcaggatgtttttagatttttgaggtgcttaaatagctattcgtatttttaaggtgtctcat
ccagaaaaaatttaatgtgcctgtaaatgttccatagaatcacaagcattaaagagttgttttatttttacataacccattaaatgtacatgtatatat
gtatatatgtatatgtgcgtgtatatacatatatatgtatacacacatgcacacacagagatatacacataccattacattgtcattcacagtcccagc
agcatgactatcacatttttgataagtgtcctttggcataaaataaaaatatcctatcagtcctttctaagaagcctgaattgaccaaaaaacatcc
ccaccaccactttataaagttgattctgctttatcctgcagtattgtttagccatcttctgctcttggtaaggttgacatagtatatgtcaatttaaaa
aataaaagtctgctttgtaaatagtaattttacccagtggtgcatgtttgagcaaacaaaaatgatgatttaagcacactacttattgcatcaaatat
gtaccacagtaagtatagtttgcaagctttcaacaggtaatatgatgtaattggttccattatagtttgaagctgtcactgctgcatgtttatcttgc
ctatgctgctgtatcttattccttccactgttcagaagtctaatatgggaagccatatatcagtggtaaagtgaagcaaattgttctaccaagacct
cattcttcatgtcattaagcaataggttgcagcaaacaaggaagagcttcttgctttttattcttccaaccttaattgaacactcaatgatgaaaag
cccgactgtacaaacatgttgcaagctgcttaaatctgtttaaaatatatggttagagttttctaagaaaatataaatactgtaaaaagttcattttat
tttatttttcagccttttgtacgtaaaatgagaaattaaaagtatcttcaggtggatgtcacagtcactattgttagtttctgttcctagcacttttaa
attgaagcacttcacaaaataagaagcaaggactaggatgcagtgtaggtttctgcttttttattagtactgtaaacttgcacacatttcaatgtgaa
acaaatctcaaactgagttcaatgtttatttgctttcaatagtaatgccttatcattgaaagaggcttaaagaaaaaaaaaatcagctgatactctt
ggcattgcttgaatccaatgtttccacctagtctttttattcagtaatcatcagtcttttccaatgtttgtttacacagatagatcttattgacccata
tggcactagaactgtatcagatataatatgggatcccagctttttttcctctcccacaaaaccaggtagtgaagttatattaccagttacagcaaaat
actttgtgtttcacaagcaacaataaatgtagattctttatactgaagctattgacttgtagtgtgttggtgaaatgcatgcaggaaaatgctgtta
ccataaagaacggtaaaccacattacaatcaagccaaaagaataaaggtttcgcttttgtttttgtatttaattgttgtctttgtttctatctttgaaa
tgccatttaaaggtagatttctatcatgtaaaaataatctatctgaaaaacaaatgtaaagaacacacattaattactataattcatctttcaattttt
tcatggaatggaagttaattaagaagagtgtattggataactactttaatattggccaaaaagctagatatggcatcaggtagactagtggaaagt
tacaaaaattaataaaaaattgactaaca);
and
SCN2A protein: NP_001035232.1 (SEQ ID NO: 8;
MAQSVLVPPGPDSFRFFTRESLAAIEQRIAEEKAKRPKQERKDEDDENGPKPNSDLEAGK
SLPFIYGDIPPEMVSVPLEDLDPYYINKKTFIVLNKGKAISRFSATPALYILTPFNPIRKLAI
KILVHSLFNMLIMCTILTNCVFMTMSNPPDWTKNVEYTFTGIYTFESLIKILARGFCLEDF
TFLRDPWNWLDFTVITFAYVTEFVDLGNVSALRTFRVLRALKTISVIPGLKTIVGALIQSV
KKLSDVMILTVFCLSVFALIGLQLFMGNLRNKCLQWPPDNSSFEINITSFFNNSLDGNGT
TFNRTVSIFNWDEYIEDKSHFYFLEGQNDALLCGNSSDAGQCPEGYICVKAGRNPNYGY
TSFDTFSWAFLSLFRLMTQDFWENLYQLTLRAAGKTYMIFFVLVIFLGSFYLINLILAVV
AMAYEEQNQATLEEAEQKEAEFQQMLEQLKKQQEEAQAAAAAASAESRDFSGAGGIG
VFSESSSVASKLSSKSEKELKNRRKKKKQKEQSGEEEKNDRVRKSESEDSIRRKGFRFSL
EGSRLTYEKRFSSPHQSLLSIRGSLFSPRRNSRASLESFRGRAKDIGSENDFADDEHSTFED
NDSRRDSLFVPHRHGERRHSNVSQASRASRVLPILPMNGKMHSAVDCNGVVSLVGGPS
TLTSAGQLLPEGTTTETEIRKRRSSSYHVSMDLLEDPTSRQRAMSIASILTNTMEELEESR
QKCPPCWYKFANMCLIWDCCKPWLKVKHLVNLVVMDPFVDLAITICIVLNTLFMAME
HYPMTEQFSSVLSVGNLVFTGIFTAEMFLKIIAMDPYYYFQEGWNIFDGFIVSLSLMELG
LANVEGLSVLRSFRLLRVFKLAKSWPTLNMLIKIIGNSVGALGNLTLVLAIIVFIFAVVGM
QLFGKSYKECVCKISNDCELPRWHMHDFFHSFLIVFRVLCGEWIETMWDCMEVAGQTM
CLTVFMMVMVIGNLVVLNLFLALLLSSFSSDNLAATDDDNEMNNLQIAVGRMQKGIDF
VKRKIREFIQKAFVRKQKALDEIKPLEDLNNKKDSCISNHTTIEIGKDLNYLKDGNGTTS
GIGSSVEKYVVDESDYMSFINNPSLTVTVPIAVGESDFENLNTEEFSSESDMEESKEKLNA
TSSSEGSTVDIGAPAEGEQPEVEPEESLEPEACFTEDCVRKFKCCQISIEEGKGKLWWNL
RKTCYKIVEHNWFETFIVFMILLSSGALAFEDIYIEQRKTIKTMLEYADKVFTYIFILEMLL
KWVAYGFQVYFTNAWCWLDFLIVDVSLVSLTANALGYSELGAIKSLRTLRALRPLRAL
SRFEGMRVVVNALLGAIPSIMNVLLVCLIFWLIFSIMGVNLFAGKFYHCINYTTGEMFDV
SVVNNYSECKALIESNQTARWKNVKVNFDNVGLGYLSLLQVATFKGWMDIMYAAVDS
RNVELQPKYEDNLYMYLYFVIFIIFGSFFTLNLFIGVIIDNFNQQKKKFGGQDIFMTEEQK
KYYNAMKKLGSKKPQKPIPRPANKFQGMVFDFVTKQVFDISIMILICLNMVTMMVETD
DQSQEMTNILYWINLVFIVLFTGECVLKLISLRYYYFTIGWNIFDFVVVILSIVGMFLAELI
EKYFVSPTLFRVIRLARIGRILRLIKGAKGIRTLLFALMMSLPALFNIGLLLFLVMFIYAIFG
MSNFAYVKREVGIDDMFNFETFGNSMICLFQITTSAGWDGLLAPILNSGPPDCDPDKDH
PGSSVKGDCGNPSVGIFFFVSYIIISFLVVVNMYIAVILENFSVATEESAEPLSEDDFEMFY
EVWEKFDPDATQFIEFAKLSDFADALDPPLLIAKPNKVQLIAMDLPMVSGDRIHCLDILF
AFTKRVLGESGEMDALRIQMEERFMASNPSKVSYEPITTTLKRKQEEVSAIIIQRAYRRY
LLKQKVKKVSSIYKKDKGKECDGTPIKEDTLIDKLNENSTPEKTDMTPSTTSPPSYDSVT
KPEKEKFEKDKSEKEDKGKDIRESKK),
NP_001035233.1 (SEQ ID NO: 9;
MAQSVLVPPGPDSFRFFTRESLAAIEQRIAEEKAKRPKQERKDEDDENGPKPNSDLEAGK
SLPFIYGDIPPEMVSVPLEDLDPYYINKKTFIVLNKGKAISRFSATPALYILTPFNPIRKLAI
KILVHSLFNMLIMCTILTNCVFMTMSNPPDWTKNVEYTFTGIYTFESLIKILARGFCLEDF
TFLRDPWNWLDFTVITFAYVTEFVNLGNVSALRTFRVLRALKTISVIPGLKTIVGALIQSV
KKLSDVMILTVFCLSVFALIGLQLFMGNLRNKCLQWPPDNSSFEINITSFFNNSLDGNGT
TFNRTVSIFNWDEYIEDKSHFYFLEGQNDALLCGNSSDAGQCPEGYICVKAGRNPNYGY
TSFDTFSWAFLSLFRLMTQDFWENLYQLTLRAAGKTYMIFFVLVIFLGSFYLINLILAVV
AMAYEEQNQATLEEAEQKEAEFQQMLEQLKKQQEEAQAAAAAASAESRDFSGAGGIG
VFSESSSVASKLSSKSEKELKNRRKKKKQKEQSGEEEKNDRVRKSESEDSIRRKGFRFSL
EGSRLTYEKRFSSPHQSLLSIRGSLFSPRRNSRASLFSFRGRAKDIGSENDFADDEHSTFED
NDSRRDSLFVPHRHGERRHSNVSQASRASRVLPILPMNGKMHSAVDCNGVVSLVGGPS
TLTSAGQLLPEGTTTETEIRKRRSSSYHVSMDLLEDPTSRQRAMSIASILTNTMEELEESR
QKCPPCWYKFANMCLIWDCCKPWLKVKHLVNLVVMDPFVDLAITICIVLNTLFMAME
HYPMTEQFSSVLSVGNLVFTGIFTAEMFLKIIAMDPYYYFQEGWNIFDGFIVSLSLMELG
LANVEGLSVLRSFRLLRVFKLAKSWPTLNMLIKIIGNSVGALGNLTLVLAIIVFIFAVVGM
QLFGKSYKECVCKISNDCELPRWHMHDFFHSFLIVFRVLCGEWIETMWDCMEVAGQTM
CLTVFMMVMVIGNLVVLNLFLALLLSSFSSDNLAATDDDNEMNNLQIAVGRMQKGIDF
VKRKIREFIQKAFVRKQKALDEIKPLEDLNNKKDSCISNHTTIEIGKDLNYLKDGNGTTS
GIGSSVEKYVVDESDYMSFINNPSLTVTVPIAVGESDFENLNTEEFSSESDMEESKEKLNA
TSSSEGSTVDIGAPAEGEQPEVEPEESLEPEACFTEDCVRKFKCCQISIEEGKGKLWWNL
RKTCYKIVEHNWFETFIVFMILLSSGALAFEDIYIEQRKTIKTMLEYADKVFTYIFILEMLL
KWVAYGFQVYFTNAWCWLDFLIVDVSLVSLTANALGYSELGAIKSLRTLRALRPLRAL
SRFEGMRVVVNALLGAIPSIMNVLLVCLIFWLIFSIMGVNLFAGKFYHCINYTTGEMFDV
SVVNNYSECKALIESNQTARWKNVKVNFDNVGLGYLSLLQVATFKGWMDIMYAAVDS
RNVELQPKYEDNLYMYLYFVIFIIFGSFFTLNLFIGVIIDNFNQQKKKFGGQDIFMTEEQK
KYYNAMKKLGSKKPQKPIPRPANKFQGMVFDFVTKQVFDISIMILICLNMVTMMVETD
DQSQEMTNILYWINLVFIVLFTGECVLKLISLRYYYFTIGWNIFDFVVVILSIVGMFLAELI
EKYFVSPTLFRVIRLARIGRILRLIKGAKGIRTLLFALMMSLPALFNIGLLLFLVMFIYAIFG
MSNFAYVKREVGIDDMFNFETFGNSMICLFQITTSAGWDGLLAPILNSGPPDCDPDKDH
PGSSVKGDCGNPSVGIFFFVSYIIISFLVVVNMYIAVILENFSVATEESAEPLSEDDFEMFY
EVWEKFDPDATQFIEFAKLSDFADALDPPLLIAKPNKVQLIAMDLPMVSGDRIHCLDILF
AFTKRVLGESGEMDALRIQMEERFMASNPSKVSYEPITTTLKRKQEEVSAIIIQRAYRRY
LLKQKVKKVSSIYKKDKGKECDGTPIKEDTLIDKLNENSTPEKTDMTPSTTSPPSYDSVT
KPEKEKFEKDKSEKEDKGKDIRESKK),
and
NP_066287.2 (SEQ ID NO: 1;
MAQSVLVPPGPDSFRFFTRESLAAIEQRIAEEKAKRPKQERKDEDDENGPKPNSDLEAGK
SLPFIYGDIPPEMVSVPLEDLDPYYINKKTFIVLNKGKAISRFSATPALYILTPFNPIRKLAI
KILVHSLFNMLIMCTILTNCVFMTMSNPPDWTKNVEYTFTGIYTFESLIKILARGFCLEDF
TFLRDPWNWLDFTVITFAYVTEFVDLGNVSALRTFRVLRALKTISVIPGLKTIVGALIQSV
KKLSDVMILTVFCLSVFALIGLQLFMGNLRNKCLQWPPDNSSFEINITSFFNNSLDGNGT
TFNRTVSIFNWDEYIEDKSHFYFLEGQNDALLCGNSSDAGQCPEGYICVKAGRNPNYGY
TSFDTFSWAFLSLFRLMTQDFWENLYQLTLRAAGKTYMIFFVLVIFLGSFYLINLILAVV
AMAYEEQNQATLEEAEQKEAEFQQMLEQLKKQQEEAQAAAAAASAESRDFSGAGGIG
VFSESSSVASKLSSKSEKELKNRRKKKKQKEQSGEEEKNDRVRKSESEDSIRRKGFRFSL
EGSRLTYEKRFSSPHQSLLSIRGSLFSPRRNSRASLFSFRGRAKDIGSENDFADDEHSTFED
NDSRRDSLFVPHRHGERRHSNVSQASRASRVLPILPMNGKMHSAVDCNGVVSLVGGPS
TLTSAGQLLPEGTTTETEIRKRRSSSYHVSMDLLEDPTSRQRAMSIASILTNTMEELEESR
QKCPPCWYKFANMCLIWDCCKPWLKVKHLVNLVVMDPFVDLAITICIVLNTLFMAME
HYPMTEQFSSVLSVGNLVFTGIFTAEMFLKIIAMDPYYYFQEGWNIFDGFIVSLSLMELG
LANVEGLSVLRSFRLLRVFKLAKSWPTLNMLIKIIGNSVGALGNLTLVLAIIVFIFAVVGM
QLFGKSYKECVCKISNDCELPRWHMHDFFHSFLIVFRVLCGEWIETMWDCMEVAGQTM
CLTVFMMVMVIGNLVVLNLFLALLLSSFSSDNLAATDDDNEMNNLQIAVGRMQKGIDF
VKRKIREFIQKAFVRKQKALDEIKPLEDLNNKKDSCISNHTTIEIGKDLNYLKDGNGTTS
GIGSSVEKYVVDESDYMSFINNPSLTVTVPIAVGESDFENLNTEEFSSESDMEESKEKLNA
TSSSEGSTVDIGAPAEGEQPEVEPEESLEPEACFTEDCVRKFKCCQISIEEGKGKLWWNL
RKTCYKIVEHNWFETFIVFMILLSSGALAFEDIYIEQRKTIKTMLEYADKVFTYIFILEMLL
KWVAYGFQVYFTNAWCWLDFLIVDVSLVSLTANALGYSELGAIKSLRTLRALRPLRAL
SRFEGMRVVVNALLGAIPSIMNVLLVCLIFWLIFSIMGVNLFAGKFYHCINYTTGEMFDV
SVVNNYSECKALIESNQTARWKNVKVNFDNVGLGYLSLLQVATFKGWMDIMYAAVDS
RNVELQPKYEDNLYMYLYFVIFIIFGSFFTLNLFIGVIIDNFNQQKKKFGGQDIFMTEEQK
KYYNAMKKLGSKKPQKPIPRPANKFQGMVFDFVTKQVFDISIMILICLNMVTMMVETD
DQSQEMTNILYWINLVFIVLFTGECVLKLISLRYYYFTIGWNIFDFVVVILSIVGMFLAELI
EKYFVSPTLFRVIRLARIGRILRLIKGAKGIRTLLFALMMSLPALFNIGLLLFLVMFIYAIFG
MSNFAYVKREVGIDDMFNFETFGNSMICLFQITTSAGWDGLLAPILNSGPPDCDPDKDH
PGSSVKGDCGNPSVGIFFFVSYIIISFLVVVNMYIAVILENFSVATEESAEPLSEDDFEMFY
EVWEKFDPDATQFIEFAKLSDFADALDPPLLIAKPNKVQLIAMDLPMVSGDRIHCLDILF
AFTKRVLGESGEMDALRIQMEERFMASNPSKVSYEPITTTLKRKQEEVSAIIIQRAYRRY
LLKQKVKKVSSIYKKDKGKECDGTPIKEDTLIDKLNENSTPEKTDMTPSTTSPPSYDSVT
KPEKEKFEKDKSEKEDKGKDIRESKK).

As used herein, the term “2′-deoxynucleoside” refers to a nucleoside comprising a 2′-H(H) deoxyfuranosyl sugar moiety. In certain embodiments, a 2′-deoxynucleoside is a 2′-β-D-deoxynucleoside and comprises a 2′-β-D-deoxyribosyl sugar moiety, which has the β-D ribosyl configuration as found in naturally occurring deoxyribonucleic acids (DNA). In certain embodiments, a 2′-deoxynucleoside may comprise a modified nucleobase or may comprise an RNA nucleobase (uracil).

As used herein, the term “2′-MOE” refers to a 2′-OCH₂CH₂OCH₃ group in place of the 2′-OH group of a furanosyl sugar moiety. A “2′-MOE sugar moiety” means a sugar moiety with a 2′-OCH₂CH₂OCH₃ group in place of the 2′-OH group of a furanosyl sugar moiety. Unless otherwise indicated, a 2′-MOE sugar moiety is in the β-D-ribosyl configuration. “MOE” means O-methoxyethyl.

As used herein, the term “2′-MOE nucleoside” refers to a nucleoside comprising a 2′-MOE sugar moiety.

As used herein, the term “5-methyl cytosine” refers to a cytosine modified with a methyl group attached at the 5 position. A 5-methyl cytosine is a modified nucleobase.

As used herein, the term “ameliorate”, used in reference to a treatment, refers to an improvement in at least one symptom or hallmark relative to the same symptom or hallmark in the absence of the treatment. In certain embodiments, amelioration is the reduction in the severity or frequency of a symptom or hallmark or the delayed onset or slowing of progression in the severity or frequency of a symptom or hallmark. In certain embodiments, the symptom or hallmark is seizures, hypotonia, sensory integration disorders, motor dysfunctions, intellectual and cognitive dysfunctions, movement and balance dysfunctions, visual dysfunctions, delayed language and speech, neurodevelopmental delays, sudden unexpected death in epilepsy, motor development delays, delayed social and language milestones, repetitive actions, uncoordinated oral movements, gastrointestinal disorders (for example, gastroesophageal reflux, diarrhea, constipation, dysmotility, and the like), or sleep problems. In certain embodiments, the seizures are focal, clonic, tonic, and generalized tonic and clonic seizures, prolonged seizures (often lasting longer than 10 minutes), or frequent seizures (for example, convulsive, myoclonic, absence, focal, obtundation status, or tonic seizures). In certain embodiments, the seizures are focal motor seizures, tonic seizures, generalized tonic-clonic seizures or myoclonic seizures.

As used herein, the term “cerebrospinal fluid” or “CSF” refers to the fluid filling the space around the brain and spinal cord. The term“artificial cerebrospinal fluid” or “aCSF” refers to a prepared or manufactured fluid that has certain properties of cerebrospinal fluid.

As used herein, the term “effective amount” of an oligomeric compound described herein refers to a quantity of the oligomeric compound sufficient to, when administered to the subject, including a human, effect beneficial or desired results, including clinical results. For example, in the context of treating a SCN2A-related disorder, e.g., a SCN2A-related disorder caused by a gain-of-function mutation, in a subject, an “effective amount” is an amount of an oligomeric compound that reduces the number of seizures experienced by the subject, as compared to the number of seizures experienced by the subject prior to the administration of the oligomeric compound. In some embodiments, an “effective amount” of an oligomeric compound is, when administered to a subject with a SCN2A-related disorder, e.g., a SCN2A-related disorder caused by a gain-of-function mutation, is sufficient to achieve a treatment response, such as a decrease in one or more symptoms, as compared to the response obtained without administration of the oligomeric compound.

As used herein, the term “gapmer” refers to a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings” or “wing segments.” In certain embodiments, the internal region is a deoxy region. The positions of the internal region or gap refer to the order of the nucleosides of the internal region and are counted starting from the 5′-end of the internal region. Unless otherwise indicated, “gapmer” refers to a sugar motif. In certain embodiments, each nucleoside of the gap is a 2′-β-D-deoxynucleoside. In certain embodiments, the gap comprises one 2′-substituted nucleoside at position 1, 2, 3, 4, or 5 of the gap, and the remainder of the nucleosides of the gap are 2′-β-D-deoxynucleosides. As used herein, the term “MOE gapmer” refers to a gapmer having a gap comprising 2′-β-D-deoxynucleosides and wings comprising 2′-MOE nucleosides. As used herein, the term “mixed wing gapmer” refers to a gapmer having wings comprising modified nucleosides comprising at least two different sugar modifications. Unless otherwise indicated, a gapmer may comprise one or more modified internucleoside linkages and/or modified nucleobases and such modifications do not necessarily follow the gapmer pattern of the sugar modifications.

As used herein, the term “internucleoside linkage” refers to the covalent linkage between contiguous nucleosides in an oligonucleotide. As used herein, the term “modified internucleoside linkage” refers to any internucleoside linkage other than a phosphodiester internucleoside linkage. The term “phosphorothioate internucleoside linkage” or “PS internucleoside linkage” refers to a modified internucleoside linkage in which one of the non-bridging oxygen atoms of a phosphodiester internucleoside linkage is replaced with a sulfur atom.

As used herein, the term “linker-nucleoside” refers to a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, the term “nucleobase” refers to an unmodified nucleobase or a modified nucleobase. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one unmodified nucleobase. A “5-methyl cytosine” is a modified nucleobase. A universal base is a modified nucleobase that can pair with any one of the five unmodified nucleobases. As used herein, “nucleobase sequence” means the order of contiguous nucleobases in a target nucleic acid or oligonucleotide independent of any sugar or internucleoside linkage modification.

As used herein, the term “nucleoside” refers to a compound or a fragment of a compound comprising a nucleobase and a sugar moiety. The nucleobase and sugar moiety are each independently unmodified or modified. As used herein, the term “modified nucleoside” refers a nucleoside comprising a modified nucleobase and/or a modified sugar moiety. Modified nucleosides include abasic nucleosides, which lack a nucleobase. The term “linked nucleosides” refers to nucleosides that are connected in a contiguous sequence (i.e., no additional nucleosides are presented between those that are linked). An oligonucleotide may be described herein as comprising a sequence of linked oligonucleosides.

As used herein, the term “oligomeric compound” refers an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound. The term “oligomeric duplex” means a duplex formed by two oligomeric compounds having complementary nucleobase sequences. Each oligomeric compound of an oligomeric duplex may be referred to as a “duplexed oligomeric compound.”

As used herein, the term “oligonucleotide” refers to a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, the term “modified oligonucleotide” refers to an oligonucleotide, wherein at least one nucleoside or internucleoside linkage is modified. As used herein, the term “unmodified oligonucleotide” refers to an oligonucleotide that does not comprise any nucleoside modifications or internucleoside modifications.

As used herein, the term “pharmaceutically acceptable carrier or diluent” refers to any substance suitable for use in administering to a subject. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water, sterile saline, sterile buffer solution or sterile artificial cerebrospinal fluid.

As used herein, the term “pharmaceutically acceptable salt” refers to any physiologically and pharmaceutically acceptable salt of a compound, e.g., an oligomeric compound. Pharmaceutically acceptable salts retain the desired biological activity of the parent compound and do not impart undesired toxicological effects thereto.

As used herein, the term “pharmaceutical composition” refers to a mixture of substances suitable for administering to a subject. For example, a pharmaceutical composition may comprise an oligomeric compound and a sterile aqueous solution.

As used herein, the term “subject” refers to any organism to which an oligomeric compound disclosed herein may be administered, e.g., for therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

In particular embodiments, a “subject” is a human subject. In further embodiments, a human subject is a pediatric subject. A pediatric subject is a child under the age of 18 years, such as under the age of 17 years, under the age of 16 years, under the age of 15 years, under the age of 14 years, under the age of 13 years, under the age of 12 years, under the age of 11 years, under the age of 10 years, under the age of 9 years, under the age of 8 years, under the age of 7 years, under the age of 6 years, under the age of 5 years, under the age of 4 years, under the age of 3 years, under the age of 2 years, under the age of 1 year, under the age of 6 months, under the age of 3 months or under the age of 1 month. In some embodiments, the pediatric subject may be 2-18 years old, e.g., 2 years old, 3 years old, 4 years old, 5 years old, 6 years old, 7 years old, 8 years old, 9 years old, 10 years old, 11 years old, 12 years old, 13 years old, 14 years old, 15 years old, 16 years old, 17 years old or 18 years old. In some embodiments, the pediatric subject may be 0-24 months old, e.g., 1 month old, 2 months old, 3 months old, 4 months old, 5 months old, 6 months old, 7 months old, 8 months old, 9 months old, 10 months old, 11 months old, 12 months old, 13 months old, 14 months old, 15 months old, 16 months old, 17 months old, 18 months old, 19 months old, 20 months old, 21 months old, 22 months old, 23 months old or 24 months old. In some embodiments, the pediatric subject is a newborn. In further embodiments, the pediatric subject is a premature newborn. In some embodiments, the newborn has low birth weight with or without prematurity.

As used herein, the term “sugar moiety” refers to an unmodified sugar moiety or a modified sugar moiety. As used herein, the term “unmodified sugar moiety” refers to a 2′-OH(H) β-D-ribosyl moiety, as found in RNA (an “unmodified RNA sugar moiety”), or a 2′-H(H) β-D-deoxyribosyl sugar moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, the terms “modified sugar moiety” or “modified sugar” refer to a modified furanosyl sugar moiety or a sugar surrogate.

As used herein, the term “symptom or hallmark” refers to any physical feature or test result that indicates the existence or extent of a disease or disorder. In certain embodiments, a symptom is apparent to a subject or to a medical professional examining or testing said subject. In certain embodiments, a hallmark is apparent upon invasive diagnostic testing, including, but not limited to, post-mortem tests. In certain embodiments, a hallmark is apparent on a brain MRI scan.

As used herein, the term “SCN2A-related disorder” refers to a class of neurological genetic diseases or disorders characterized by aberrant function of SCN2A. SCN2A-related disorders include, for example, epilepsy, pediatric epilepsy, benign familial neonatal/infantile seizures, severe early-onset epilepsy, epileptic encephalopathy, early infantile epileptic encephalopathy (i.e., early onset epileptic encephalopathy), late seizure onset epileptic encephalopathy, Ohtahara syndrome, infantile spasm syndrome (i.e., West syndrome), Lennox-Gastaut syndrome, generalized epilepsy with febrile seizures, migrating partial epilepsy of infancy (i.e., epilepsy of infancy with migrating focal seizures), infantile spasms, autism spectrum disorder, movement disorder, and drug-resistant epilepsies. In some embodiments, a SCN2A-related disorder may be caused by a gain-of-function SCN2A mutation. SCN2A-related disorders that may be caused by a gain-of-function SCN2A mutation include, for example, Ohtahara syndrome, epilepsy of infancy with migrating focal seizures, developmental and epileptic encephalopathy (DEE), including early seizure onset epileptic encephalopathy (EE). In some embodiments, a gain-of-function mutation in SCN2A that is associated with a SCN2A-related disorder includes a mutation selected from the group consisting of L210Q, A263V, E430A, R1882Q, G879R, Q1479H, V423L, G1593R, K1502N, V1601L, G211D, S1780I, D343H, and c.3986C>A p.(A1329D).

### Oligomeric Compound

Methods of the present disclosure comprise administering to a subject with a SCN2A-related disorder an oligomeric compound that comprises a 6-10-4 MOE gapmer having a sequence (from 5′ to 3′) of CCACGACATATTTTTCTACA (SEQ ID NO: 3); wherein each of nucleosides 1-6 and 17-20 (from 5′ to 3′) are 2′-MOE nucleosides and each of nucleosides 7-16 are 2′-β-D-deoxynucleosides; wherein the internucleoside linkages between nucleosides 2 to 3, 3 to 4, 4 to 5, 5 to 6, 6 to 7, and 17 to 18 are phosphodiester internucleoside linkages, the internucleoside linkages between nucleosides 1 to 2, 7 to 8, 8 to 9, 9 to 10, 10 to 11, 11 to 12, 12 to 13, 13 to 14, 14 to 15, 15 to 16, 16 to 17, 18 to 19, and 19 to 20 are phosphorothioate internucleoside linkages; and wherein each cytosine is a 5-methyl cytosine.

In some embodiments, the oligomeric compound comprises a modified oligonucleotide represented by the following chemical notation: ^{m}Cₑₛ^{m}CₑₒAₑₒ^{m}CₑₒGₑₒAₑₒ^{m}C_{ds}A_{ds}T_{ds}A_{ds}T_{ds}T_{ds}T_{ds}T_{ds}T_{ds}^{m}C_{ds}TₑₒAₑₛ^{m}CₑₛAₑ (SEQ ID NO: 3), wherein:
A=an adenine nucleobase,
^{m}C=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

In some embodiments, the oligomeric compound is a modified oligonucleotide represented by the following chemical structure:

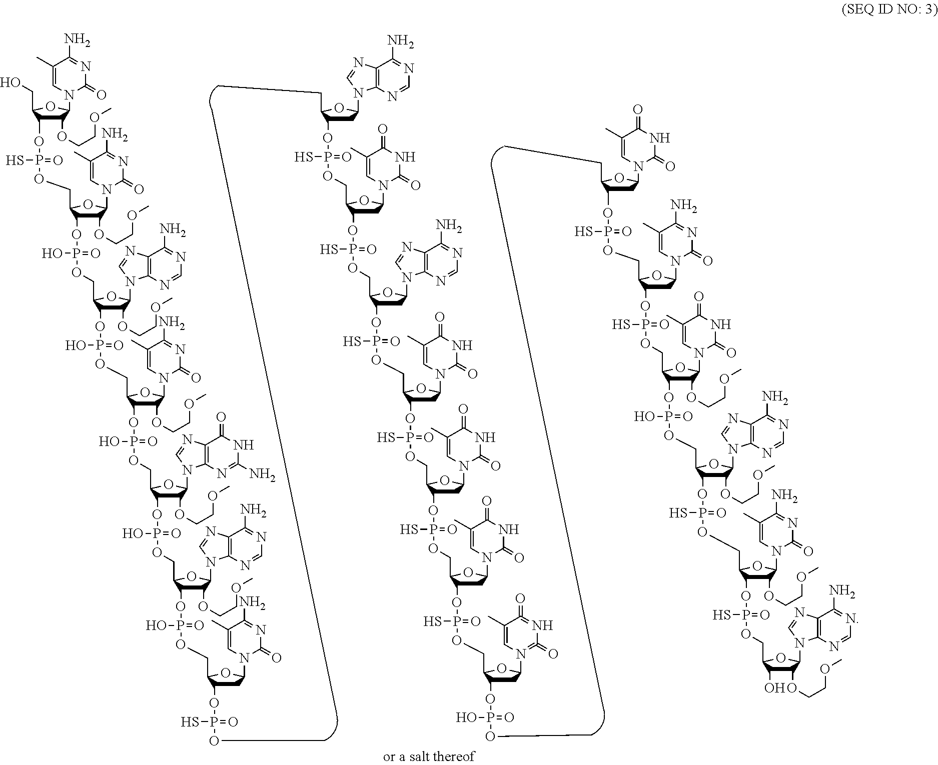

The modified oligonucleotide may be a sodium salt or a potassium salt. In one embodiment, the modified oligonucleotide is a sodium salt.

In some embodiments, the modified oligonucleotide may be represented by the following chemical structure:

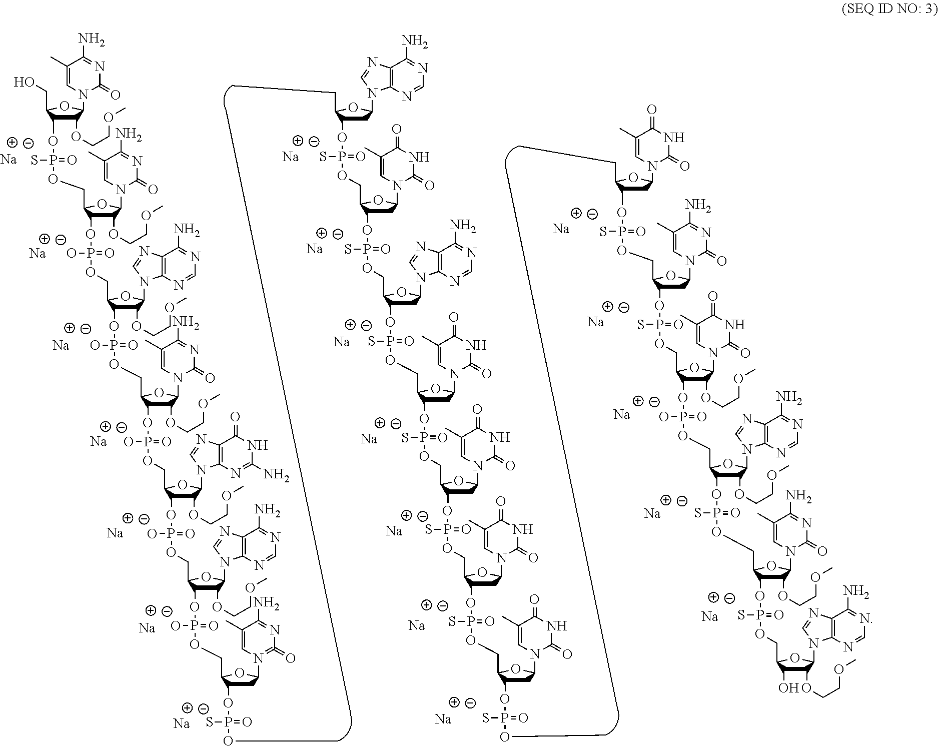

In some embodiments, the oligomeric compound of the present disclosure may also be referred to herein as “Compound 1” or “elsunursen”.

In the context of the present disclosure, the oligomeric compound of the disclosure may be administered as a part of a pharmaceutical composition comprising the oligomeric compound and a pharmaceutically acceptable carrier or diluent. In certain embodiments, a pharmaceutical composition comprises or consists of a sterile saline solution and the oligomeric compound. In certain embodiments, the sterile saline is pharmaceutical grade saline. In certain embodiments, a pharmaceutical composition comprises or consists of the oligomeric compound and sterile water. In certain embodiments, the sterile water is pharmaceutical grade water. In certain embodiments, a pharmaceutical composition comprises or consists of the oligomeric compound and phosphate-buffered saline (PBS). In certain embodiments, the sterile PBS is pharmaceutical grade PBS. In certain embodiments, the pharmaceutical composition comprises or consists of the oligomeric compound and artificial cerebrospinal fluid (“artificial CSF” or “aCSF”). In certain embodiments, the artificial cerebrospinal fluid is pharmaceutical grade.

In certain embodiments, the pharmaceutical composition may comprise the oligomeric compound of the disclosure and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In the context of the present disclosure, the oligomeric compound may be administered to a subject by an injection (e.g., intravenously, subcutaneously, intramuscularly, intrathecally (IT), intracerebroventricularly (ICV), intraneurally, perineurally, etc.). In some embodiments, the oligomeric compound is administered to a subject intrathecally.

### Methods of Treating SCN2A-Related Disorders

Disclosed herein are methods of reducing number of seizures experienced by a subject with a SCN2A-related disorder caused by a gain-of-function mutation in SCN2A gene that comprise administering to the subject an oligomeric compound of the present disclosure.

The present disclosure also provides methods of reducing frequency of seizures experienced by a subject with a SCN2A-related disorder that comprise administering to the subject an effective amount of an oligomeric compound of the present disclosure. In some embodiments, the term “frequency of seizures” refers to the number of seizures experienced by a subject in a given time period, e.g., a time period of a day, a week, 14 days, 28 days, a month, 3 months, 6 months or a year.

The present disclosure also provides methods of reducing the number of average daily seizures experienced by a subject with a SCN2A-related disorder that comprise administering to the subject an effective amount of an oligomeric compound of the present disclosure.

The present disclosure also provides methods of increasing the number of seizure-free days experienced by a subject with a SCN2A-related disorder that comprise administering to the subject an effective amount of an oligomeric compound of the present disclosure.

The present disclosure also provides methods of reducing seizure burden experienced by a subject with a SCN2A-related disorder or experienced by a caregiver of a subject with a SCN2A-related disorder that comprise administering to the subject an effective amount of an oligomeric compound of the present disclosure. In some embodiments, the term “seizure burden”, as used herein, encompasses seizure frequency, seizure severity and burdens associated with seizure unpredictability. Burdens associated with seizures are discussed, e.g., in Berg et al., Epilepsia Open 2019, 4:293-301, the entire contents of which are hereby incorporated herein by reference.

In some embodiments, methods of the present disclosure allow achieving a reduction in seizures from baseline on top of, e.g., additional to the reduction in seizures based upon, best available standard of care. In some embodiments, a subject being administered Compound 1 in the context of the present disclosure in combination with standard of care therapy experiences a greater reduction in the number of seizures, e.g., the number of average daily seizures, than a subject who is administered standard of care therapy but is not administered Compound 1. In some embodiments, a subject being administered Compound 1 in the context of the present disclosure in combination with standard of care therapy experiences a greater reduction in the frequency of seizures than a subject who is administered standard of care therapy but is not administered Compound 1. In some embodiments, a subject being administered Compound 1 in the context of the present disclosure in combination with standard of care therapy experiences a greater increase in the number of seizures-free days than a subject who is administered standard of care therapy but is not administered Compound 1. In some embodiments, a subject being administered Compound 1 in the context of the present disclosure in combination with standard of care therapy experiences a greater decrease in seizure burden than a subject who is administered standard of care therapy but is not administered Compound 1.

The term “standard of care therapy”, as used herein, encompasses any therapy (other than Compound 1) that is useful for treating a SCN2A-related disorder. In some embodiments, the standard of care therapy may comprise one or more sodium channel blocking medications, e.g., carbamazepine, oxcarbazepine, lamotrigine, valproate or topiramate.

Also disclosed herein are methods of treating a subject with a SCN2A-related disorder caused by a gain-of-function mutation in SCN2A gene that comprise administering to the subject an oligomeric compound of the present disclosure at a dose of about 0.1 mg to about 20 mg.

The methods disclosed herein may be used to ameliorate one or more symptoms of early-onset DEE, including, for example, seizures, limitations in communication, such as delayed language and speech; autonomic dysfunction; developmental delay; gastrointestinal abnormalities; movement disorders, such as choreoathetosis, dystonia, ataxia; anxiety; sensory issues; urinary retention problems; irritability; sleep problems (e.g., inability to fall asleep and inability to stay asleep); and behavior issues. Seizures include focal, clonic, tonic, and generalized tonic and clonic seizures, prolonged seizures (often lasting longer than 10 minutes), and frequent seizures.

The methods disclosed herein for treating a subject with a SCN2A-related disorder may have a disease-modifying effect on the subject. The disease-modifying effect may include one or more of, e.g., a reduction in seizure frequency experienced by a subject, an increase in the number of seizure-free days experienced by a subject; and an increase in survival of a subject, as compared to a subject who is administered standard of care therapy but is not administered Compound 1, or as compared to a subject who is not treated. In some embodiments, the disease modifying effect may also include a normalized developmental curve which includes one or more of, e.g., an increased ability of a subject to nurse, to interact socially, to process and/or manage outside stimuli, to grow and/or to develop language and/or speech, as compared to a subject who is administered standard of care therapy but is not administered Compound 1, or as compared to a subject who is not treated.

In some embodiments, methods of the present disclosure reduce the number of seizures experienced by the subject, as compared to the number of seizures experienced by the subject prior to the administration of the oligomeric compound. In some embodiments, seizures may include one or more of focal, clonic, tonic, and generalized tonic and clonic seizures, prolonged seizures (often lasting longer than 10 minutes), and frequent seizures. In some embodiments, seizures may include one or more of focal motor seizures, tonic seizures, generalized tonic-clonic seizures and myoclonic seizures.

In certain aspects, methods of the present disclosure comprise a dose escalation phase (or a titration phase) and a maintenance phase. Typically, the dose escalation phase is used to determine a maximum dose for the subject and the maintenance phase is used to administer the maximum dose to the subject on an ongoing basis. In certain aspects, the method does not include a dose escalation phase and a selected dose (also referred to herein as a maintenance dose) is administered to the subject on an ongoing basis.

In some embodiments, the oligomeric compound is administered to a subject in the context of the present disclosure at a dose of about 0.1 mg to about 20 mg, e.g., about 0.1 mg to about 0.5 mg, about 0.1 mg to about 1 mg, about 1 mg to about 5 mg, about 0.5 mg to about 8 mg, about 0.5 mg to about 10 mg, about 1 mg to about 8 mg, about 1 mg to about 10 mg, about 2 mg to about 10 mg or about 5 mg to about 15 mg. In some embodiments, the oligomeric compound is administered at a dose of about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.5 mg, about 2 mg, about 2.5 mg, about 3 mg, about 3.5 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg or about 20 mg. In some embodiments, the oligomeric compound is administered at a dose of about 0.5 mg, about 1 mg, about 1.5 mg, about 2.0 mg, about 2.5 mg, about 3 mg, about 3.5 mg, about 4 mg, about 5 mg, about 8 mg, about 12 mg or about 15 mg. In one embodiment, the oligomeric compound is administered at a dose of about 1 mg. In some embodiments, the oligomeric compound is administered at a dose of about 8 mg.

In some embodiments, the oligomeric compound is administered at a dose of at least 0.25 mg. In some embodiments, the oligomeric compound is administered at a dose of at least 0.5 mg. In some embodiments, the oligomeric compound is administered at a dose of at least 0.75 mg. In some embodiments, the oligomeric compound is administered at a dose of at least 1 mg. In some embodiments, the oligomeric compound is administered at a dose of at least 0.25 mg, at least 0.5 mg, at least 0.75 mg, at least 1 mg, at least 1.5 mg, at least 2 mg, at least 2.5 mg, at least 3 mg, at least 4 mg, at least 5 mg, at least 6 mg, at least 7 mg, at least 8 mg, at least 9 mg, at least 10 mg, at least 11 mg, at least 12 mg, at least 13 mg, at least 14 mg, at least 15 mg, at least 16 mg, at least 17 mg, at least 18 mg, at least 19 mg, or at least 20 mg.

In one aspect, the dosage of the oligomeric compound may be titrated one or more times. In certain embodiments, the dosage is increased 0.5-fold to 10-fold between each titrated dose, 1-fold to 10-fold or 1.25-fold to 5-fold between each titrated dose. In certain embodiments, the dosage is increased 1.25-fold to 2-fold, 1.5-fold to 3-fold, 1.5-fold to 2.5-fold, or 1.5-fold to 2-fold between each titrated dose. In certain embodiments, the dosage is increased 1.25-fold to 1.75-fold between each titrated dose. In certain embodiments, the dosage is increased 2-fold, up to 8 mg, followed by no more than a 1.5-fold increase for all subsequent doses.

In various embodiments, the dosage may be titrated upwardly or downwardly in the medical judgment of a clinician based upon any clinical factors such as for example, patient response, or lack of response, at a particular dosage, laboratory results (e.g., blood count, ECG results, brain scan results, results of pharmacokinetic assessments, etc.), patient illness or adverse events.

Downward titration may occur any number of times by any factor such as decreasing the dosage 0.5-fold to 10-fold between each downwardly titrated dose, decreasing the dosage 1-fold to 10-fold or 1.25-fold to 5-fold between each downwardly titrated dose. In certain embodiments, the dosage is decreased 1.25-fold to 2-fold, 1.5-fold to 3-fold, 1.5-fold to 2.5-fold, or 1.5-fold to 2-fold between each downwardly titrated dose. In certain embodiments, the dosage is decreased 1.25-fold to 1.75-fold between each titrated dose. In certain embodiments, the dosage is decreased 0.5-fold, 1-fold, or 2-fold. In an embodiment, downward titration may include immediate cessation of dosing or a pause in dosing.

In various embodiments upward titration may be followed by downward titration. In other embodiments, downward titration may be followed by upward titration.

In certain embodiments, during the dose escalation phase, the dosage of the oligomeric compound may be titrated from an initial dose of about 1 mg to a maximum dose of at least about 1.5 mg, about 2 mg, about 3 mg, about 3.5 mg, about 4 mg, about 5 mg, about 8 mg, about 10 mg, about 12 mg, about 15 mg, about 18 mg, about 20 mg, about 25 mg, about 30 mg, about 32 mg, about 35 mg, about 40 mg, about 45 mg, about 50 about mg, about 55 mg, about 60 mg, about 65 mg, about 70 mg, about 75 mg, about 80 mg, about 85 mg, about 90 mg, about 100 mg, or more. In certain embodiments, during the dose escalation phase, the dosage of the oligomeric compound is titrated from an initial dose of about 0.25 mg, about 0.5 mg, about 0.75 mg, about 1 mg, about 1.5 mg, about 2 mg, about 3 mg, about 4 mg, about 5 mg, about 6 mg, about 7 mg, about 8 mg, about 9 mg, about, 10 mg, about 11 mg, about 12 mg, about 13 mg, about 14 mg, or about 15 mg to a maximum dose of at least about 20 mg, about 25 mg, about 30 mg, about 35 mg, about 40 mg, about 45 mg, about 50 mg, about 60 mg, about 65 mg, about 70 mg, about 75 mg, about 80 mg, about 85 mg, about 90 mg, about 95 mg, about 100 mg, or more. In certain embodiments, the maximum dose is about 4-100 mg, about 4-50 mg, about 4-25 mg, about 4-15 mg, about 1-10 mg, about 1.5-10 mg, about 4-8 mg, about 1 to 5 mg, about 1.5 to 5 mg, about 1.5 to 8 mg, about 8-100 mg, about 8-75 mg, about 8-50 mg, about 8-25 mg, or about 8-15 mg. In certain embodiments, the maximum dose is about 15-100 mg, about 15-75 mg, about 15-50 mg, about 15-40 mg, about 15-30 mg, about 15-25 mg. In certain embodiments, during the dose escalation phase, the dosage of the oligomeric compound is titrated from an initial dose of about 1 mg to a maximum dose of at least about 15 mg. In certain embodiments, during the dose escalation phase, the dosage of the oligomeric compound is titrated from an initial dose of about 1 mg to a maximum dose of about 32 mg. In certain embodiments, during the dose escalation phase, the dosage of the oligomeric compound is titrated from an initial dose of about 1 mg to a maximum dose of about 32-64 mg. In certain embodiments, the dosage of the oligomeric compound is titrated from an initial dose of about 1 mg to a maximum dose of at least 64 mg.

In certain embodiments, the dosage of the oligomeric compound may be administered from an initial dose of about 1 mg to a cumulative dose of at least about 5 mg, about 7 mg, about 8 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 500 mg, about 1 g, about 10 g, or about 54 grams or more. In certain embodiments, the dosage of the oligomeric compound is administered from an initial dose of about 1 mg, about 1.5 mg, about 2 mg, about 2.5 mg, about 3 mg, about 3.5 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 25 mg, or about 30 mg to a cumulative dose of at least about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 500 mg, about 1 g, about 10 g, or about 54 grams or more.

In certain embodiments, the cumulative dose is about 40-200 mg, about 50-200 mg, about 50-175 mg, about 50-150 mg, about 50-125 mg, about 50-100 mg, about 50-75 mg, about 40-50 mg, about 75-200 mg, about 75-175 mg, about 75-150 mg, about 75-125 mg, about 75-100 mg, about 100-200 mg, about 100-175 mg, about 100-150 mg, about 100-125 mg, about 125-200 mg, about 125-175 mg, about 125-150 mg, about 150-200 mg, or about 150-175 mg. In certain embodiments, the dosage of the oligomeric compound is administered from an initial dose of about 1 mg to a cumulative dose of at least about 40 mg or 50 mg. In certain embodiments, the dosage of the oligomeric compound is administered from an initial dose of about 1 mg to a cumulative dose of at least about 100 mg. In certain embodiments, the dosage of the oligomeric compound is administered from an initial dose of about 1 mg to a cumulative dose of at least about 40-100 mg, 50-100 mg, 100 mg to 200 mg, 200 mg to 500 mg, about 500 mg to about 1 g, about 1 g to about 10 g, about 10 g to about 540 g.

In some embodiments, the dosing of the oligomeric compound, including dose escalations and reductions during treatment, is determined by a clinician in light of available information and relevant considerations. The relevant considerations may include any considerations deemed relevant by a clinician, such as, by way of non-limiting example, overall health of patient, response to drug treatment, infectious disease state and management, vital signs, seizure counts and/or test results, e.g., blood count, ECG results, brain scan results, results of pharmacokinetic assessments, etc.

In one aspect, the maximum dose is reached after administration of multiple titrated doses of the oligomeric compound. In certain embodiments, the maximum dose of the dose escalation phase is reached after administration of 2-20 titrated doses of the oligomeric compound, including after 2, 3, 4, 5, 6, 7, 8, 9, 10, 11, 12, 13, 14, 15, 16, 17, 18, 19, or 20 titrated doses of the oligomeric compound. In certain embodiments, the maximum dose of the dose escalation phase is reached after administration of 2-4, 2-10, 4-16, 4-12, 4-10, 4-8, 5-15, 5-12, 5-10, 5-8, 6-12, 6-10, 8-12, or 8-10 titrated doses of the oligomeric compound. In certain embodiments, the maximum dose of the dose escalation phase is reached after administration of up to about 4 to about 12 titrated doses of the oligomeric compound. In other embodiments, no titration is needed to reach the maximum dose, e.g., dose 1 is the maximum dose.

In one aspect, each administration of the titrated dose of the oligomeric compound is separated by about 3-20 weeks. In certain embodiments, each administration of the titrated dose of the oligomeric compound is separated by about 1 week, 2, 3, 4, 5, 6, 7, 8, 9, 10, 11, 12, 13, 14, 15, 16, 17, 18, 19, or 20 weeks. In certain embodiments of the dose escalation phase, the titrated dose of the oligomeric compound is administered about every 4-6 weeks. In certain embodiments, each administration of the titrated dose of the oligomeric compound is separated by at least about 6 weeks. In certain embodiments, each administration of the titrated dose of the oligomeric compound is separated by at least about 2-4 weeks. In other embodiments, each administration of the titrated dose of the oligomeric compound is separated by at least about 2 weeks, at least about 3 weeks, or at least about 4 weeks. In certain embodiments, a titrated dose of the oligomeric compound is administered no more frequently than every 6 weeks. In other embodiments, a titrated dose of the oligomeric compound is administered no more frequently than every 4 weeks. In other embodiments, a titrated dose of the oligomeric compound is administered no more frequently than every 3 weeks. In other embodiments, a titrated dose of the oligomeric compound is administered no more frequently than every 2 weeks.

In some embodiments, the oligomeric compound is administered to a subject every week. In some embodiments, the oligomeric compound is administered to a subject every 2 weeks. In some embodiments, the oligomeric compound is administered to a subject every 3 weeks. In some embodiments, the oligomeric compound is administered to a subject every 4 weeks. In some embodiments, the oligomeric compound is administered to a subject every 6 weeks. In some embodiments, the oligomeric compound is administered to a subject every 8 weeks. In some embodiments, the oligomeric compound is administered to a subject every 10 weeks. In some embodiments, the oligomeric compound is administered to a subject every 12 weeks. In some embodiments, the oligomeric compound is administered to a subject every month.

In certain embodiments, during the dose escalation phase, the dosage of the oligomeric compound is titrated from an initial dose of about 1 mg to a maximum dose of about 15 mg. In certain embodiments, during the dose escalation phase, the dosage of the oligomeric compound is titrated from a first dose of about 1 mg to a second dose of about 2 mg, and optionally to a third dose of about 4 mg, and optionally to a fourth dose of up to about 8 mg, and optionally to a fifth dose of up to about 12 mg, and optionally to a sixth dose of up to about 15 mg. In certain embodiments, the fourth dose of about 8 mg is titrated to a fifth dose of about 15 mg. In certain embodiments, 2-6 weeks separate the administration of each titrated dose. In other embodiments, the titration can continue up to a maximum dose of 100 mg.

In one aspect, the method of treatment further comprises administering a maintenance dose during a maintenance phase. In certain embodiments, the maintenance phase follows the dose escalation phase. In certain embodiments, the maintenance dose is the maximum dose following the dose escalation phase. In other embodiments, the maintenance dose is selected and administered to a subject without the subject undergoing a dose escalation phase.

In certain embodiments, the maintenance dose is administered about every 16-20 weeks or every 2-20 weeks. In certain embodiments, the maintenance dose is administered about every 1, 2, 3, 4, 5, 6, 7, 8, 9, 10, 11, 12, 13, 14, 15, 16, 17, 18, 19, or 20 weeks. In certain embodiments, the maintenance dose is administered about every 1-4 weeks, 1-6 weeks, 1-8 weeks, 1-16 weeks, 2-16 weeks, 2-8 weeks, 6-18 weeks, 6-16 weeks, 6-14 weeks, 6-12 weeks, 6-10 weeks, 8-18 weeks, 8-16 weeks, 8-14 weeks, 8-12 weeks, 8-10 weeks, 10-18 weeks, 10-16 weeks, 10-14 weeks, or 10-12 weeks. In certain embodiments, the maintenance dose is administered about every 12 weeks.

In one aspect of the methods of treatment, the maintenance dose is administered after the maximum dose, as described herein, has been reached during the dose escalation phase. In certain embodiments, the maintenance dose is administered after a maximum dose of about 1-15 mg, e.g., a maximum dose of about 1 mg, about 1.5 mg, about 2 mg, about 2.5 mg, about 3 mg, about 4 mg, about 5 mg, about 6 mg, about 7 mg, about 8 mg, about 10 mg, about 12 mg or about 15 mg has been reached. In certain embodiments, the maintenance dose is administered after the maximum dose of about 15 mg has been reached. In certain embodiments, the maintenance dose is administered after a maximum dose of greater than 15 mg has been reached. In certain embodiments, the maintenance dose is administered after a maximum dose of about 15-32 mg has been reached. In certain embodiments, the maintenance does is administered after a maximum dose of about 30-50 mg has been reached. In certain embodiments, the maintenance dose is administered after a maximum dose of greater than 50 mg has been reached.

In one aspect, multiple maintenance doses are administered. In certain embodiments each administration of the maintenance dose is separated by about 2-20 weeks. In certain embodiments, each administration of the maintenance dose is separated by about 2, 3, 4, 5, 6, 7, 8, 9, 10, 11, 12, 13, 14, 15, 16, 17, 18, 19, or 20 weeks. In various embodiments, each administration of the maintenance dose is separated by about a month. In certain embodiments, each administration of the maintenance dose is separated by about 8-16, 8-14, 8-12, 8-10, 10-16, 10-14, or 10-12 weeks. In certain embodiments, the maintenance dose is administered at least about every 12 weeks.

In one aspect, maintenance doses are administered over a period of 6 months or greater. In certain embodiments, maintenance doses are administered for the remainder of a subject's life. In certain embodiments, maintenance doses are administered for at least 1-50 years. In certain embodiments, maintenance doses are administered for at least 1, 2, 3, 4, 5, 6, 7, 8, 9, or 10 years. In certain embodiments, maintenance doses are administered for at least 2-50 years, 2-40 years, 2-35 years, 2-30 years, 2-25 years, 2-20 years, 2-15 years, 2-10 years, or 2-5 years. In certain embodiments, maintenance doses are administered for at least 2 years.

In one aspect, the method of treatment comprises inhibiting the expression of SCN2A in neuronal cells in the subject. In various aspects, the method of treatment comprises inhibiting the expression of SCN2A mRNA in the subject by about 10% to about 90%, about 35% to about 80%, about 20%, about 25%, about 30%, about 35%, about 40%, about 45%, about 50%, about 55%, about 60%, about 65%, about 70%, about 75%, about 80%, about 85%, about 90%, about 95%, or more than about 95% but less than 100%. In other aspects, about 25% to about 50% or about 35% to about 40% knockdown of SCN2A mRNA is achieved.

In an aspect, the subject has been diagnosed with early-onset DEE prior to one year of age. In another aspect, the subject has been diagnosed with early-onset DEE prior to three months of age. In certain embodiments, the subject has been diagnosed with early-onset DEE prior to two months or prior to one month of age. In certain embodiments, the subject has been diagnosed with early-onset DEE in utero. Typically, the diagnosis comprises determining that the subject carries an SCN2A mutation prior to administering the oligomeric compound. Any SCN2A mutation can be used to diagnose early-onset DEE, including any known SCN2A mutation and any SCN2A mutation identified in the future. In certain embodiments, the SCN2A mutation comprises one or more of A263V, E430A, E430G, R1882Q, G879R, G1593R, K1502N, V1601L, G211D, S1780I, D343H, R1626Q, G882E, M1545V, L210Q, Q1479H, N1662D, F1597L, V423L, A215T, I891T, A1329D, or a combination thereof. In some embodiments, the SCN2A mutation comprises one or more of Q1531K, L1563V, E1321K, Y1589C, M252V, R223E, L1330F, V208E, R36G, R1882G, D343G, V261L, F1651C, R1319Q, A263V, Q383E, V1325I, K908E, V261M, S987I, R1629H, R1882Q, M1338T, E999K, R856Q, V423L, S1336Y, R1626Q, G882E, N212D, E1211K, D195G, L1342P, R220Q, R853Q, R1435*, K503fs*, R937C, or a combination thereof. In some embodiments, the SCN2A mutation comprises one or more of L210Q, A263V, E430A, R1882Q, G879R, Q1479H, V423L, G1593R, K1502N, V1601L, G211D, S1780I, D343H and A1329D, or a combination thereof. In some embodiments, the SCN2A mutation comprises A1329D.

In one aspect, the subject is a human or non-human animal. In certain embodiments, the subject is a human. In certain embodiments, the subject is a human from 2 years old to 18 years old. In other embodiments, the subject is a human older than 2 years old or younger than 18 years old. In other embodiments, the subject is a human older than 18 years old.

In certain embodiments, the subject is a human younger than 2 years old. In certain embodiments, the subject is a human infant. In further embodiments, the infant is 6 months or younger, 5 months or younger, 4 months or younger, 3 months or younger, 2 months or younger or 1 month or younger.

In certain embodiments, the subject is a human newborn. In further embodiments, the newborn is a full term newborn, i.e., born after 39 or more weeks of gestation. In other embodiments, the newborn is a premature newborn, i.e., born after less than 39 weeks of gestation. In some embodiments, the subject is a human premature newborn born after less than 38 weeks, less than 37 weeks, less than 36 weeks, less than 35 weeks, less than 34 weeks, less than 33 weeks, less than 32 weeks, or less than 31 weeks, less than 30 weeks, or less than 28 weeks of gestation. In various embodiments, the newborn has a low birth weight regardless of gestational age.

An oligomeric compound described herein may be administered to a subject in the context of the present invention in combination with another agent or therapy, e.g., an antiepilepsy agent. Non-limiting examples of the anti-epilepsy agents include brivaracetam, carbamazepine, clobazam, clonazepam, diazepam, divalproex, eslicarbazepine, ethosuximide, ezogabine, felbamate, gabapentin, lacosamide, lamotrigine, levetiracetam, lorazepam, oxcarbezepine, permpanel, phenobarbital, phenytoin, pregabalin, primidone, rufinamide, tigabine, topiramate, valproic acid, vigabatrin, zonisamide, and cannabidiol. In an embodiment, an oligomeric compound is administered in combination with carbamazepine.

In addition to intrathecal administration, the oligomeric compounds described herein may also be administered, for example, by oral, parenteral, intracerebroventricular, intraparenchymal, buccal, sublingual, nasal, rectal, patch, pump, or transdermal administration. Parenteral administration includes intravenous, intraperitoneal, subcutaneous, intramuscular, transepithelial, nasal, intrapulmonary, intrathecal, intracisternal, intracerebroventricular, intraparenchymal, rectal, and topical modes.

Other SCN2A disorders can be treated according to the methods disclosed in this application include developmental or epileptic encephalopathy (DEE), such as, Ohtahara Syndrome; epilepsy with migrating focal seizures of infancy (EIMFS); infantile and childhood DEE, for example West Syndrome and Lennon-Gastaut Syndrome; Dravet Syndrome; Idiopathic/Generic Generalized Epilepsies (IGE/GGE); Temporal Lobe Epilepsy; Myoclonic Astatic Epilepsy (MAE); Migrating Partial Epilepsy of Infancy (MMPSI); and familial hemiplegic migraines, with or without epilepsy. In certain embodiments, the SCN2A-related disorder is late seizure onset epileptic encephalopathy. In certain embodiments, the SCN2A-related disorder is Benign Familial Neonatal-Infantile Seizures. In certain embodiments, the SCN2A-related disorder is an intellectual disability (ID). In certain embodiments, the SCN2A-related disorder is an autism spectrum disorder (ASD).

The methods of treatment disclosed herein may be used to ameliorate one or more symptoms of SCN2A disorders, including seizures, hypotonia, sensory issues, such as sensory integration disorders, motor development delays and dysfunctions, intellectual and cognitive dysfunctions, movement and balance dysfunctions, visual dysfunctions, delayed language and speech, gastrointestinal disorders, neurodevelopmental delays, and sleep problems. Seizures include focal, clonic, tonic, and generalized tonic and clonic seizures, prolonged seizures (often lasting longer than 10 minutes), and frequent seizures.

In some embodiments, administration of an oligomeric compound to a subject in need thereof in the context of the present disclosure results in a decrease in the frequency of seizures experienced by the subject, as compared to the frequency of seizures experienced by the subject prior to the administration of the oligomeric compound. In some embodiments, administration of an oligomeric compound to a subject in need thereof in the context of the present disclosure results in a decrease of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75% at least about 80%, at least about 85%, at least about 90% or at least about 95% in the frequency of seizures experienced by the subject, as compared to the frequency of seizures experienced by the subject prior to the administration of the oligomeric compound. In some embodiments, a decrease in the frequency of seizures in the subject is observed at least 1 week, at least 2 weeks, at least 4 weeks, at least 6 weeks, at least 8 weeks, at least 10 weeks, at least 12 weeks, at least 14 weeks or at least 16 or more weeks after the start of the administration of an oligomeric compound. In some embodiments, a decrease in frequency of seizures experienced by the subject is observed within 28 days after the start of administration of the oligomeric compound. In some embodiments, the term “frequency of seizures” refers to the number of seizures experienced by a subject in a given time period, e.g., a time period of 28 days.

In some embodiments, administering an oligomeric compound to a subject with a SCN2A-related disorder in the context of the present disclosure results in a decrease in the number of average daily seizures experienced by the subject, as compared to the number of average daily seizures experienced by the subject prior to administering the oligomeric compound. In some embodiments, administering an oligomeric compound to a subject in need thereof in the context of the present disclosure results in a decrease of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% in the number of average daily seizures experienced by the subject, as compared to the number of average daily seizures experienced by the subject prior to administering an oligomeric compound. In some embodiments, a decrease in the number of average daily seizures experienced by the subject is observed at least 1 week, at least 2 weeks, at least 4 weeks, at least 6 weeks, at least 8 weeks, at least 10 weeks, at least 12 weeks, at least 14 weeks or at least 16 or more weeks after the start of the administration of an oligomeric compound. In other embodiments, a decrease in the number of average daily seizures is observed within 24 hours after the start of administration of an oligomeric compound. In some embodiments, a decrease in the number of average daily seizures is observed within 28 days after the start of administration of the oligomeric compound. In some embodiments, a decrease in the number of average daily seizures is observed following administration of a single dose of the oligomeric compound. In some embodiments, a decrease in the number of average daily seizures is observed following administration of multiple doses of the oligomeric compound, i.e., two doses of the oligomeric compound, three doses of the oligomeric compound, or four or more doses of the oligomeric compound.

In some embodiments, administering of an oligomeric compound to a subject with a SCN2A-related disorder in the context of the present disclosure results in a decrease in the frequency of focal motor seizures experienced by the subject, as compared to the frequency of focal motor seizures experienced by the subject prior to the administration of the oligomeric compound. In some embodiments, administration of an oligomeric compound to a subject in need thereof in the context of the present disclosure results in a decrease of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75% at least about 80%, at least about 85%, at least about 90% or at least about 95% in the frequency of focal motor seizures experienced by the subject, as compared to the frequency of focal motor seizures experienced by the subject prior to the administration of the oligomeric compound.

In some embodiments, administering of an oligomeric compound to a subject with a SCN2A-related disorder in the context of the present disclosure results in a decrease in the frequency of tonic seizures experienced by the subject, as compared to the frequency of tonic seizures experienced by the subject prior to the administration of the oligomeric compound. In some embodiments, administration of an oligomeric compound to a subject in need thereof in the context of the present disclosure results in a decrease of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75% at least about 80%, at least about 85%, at least about 90% or at least about 95% in the frequency of tonic seizures experienced by the subject, as compared to the frequency of tonic seizures experienced by the subject prior to the administration of the oligomeric compound.

In some embodiments, administering of an oligomeric compound to a subject with a SCN2A-related disorder in the context of the present disclosure results in a decrease in the frequency of myoclonic seizures experienced by the subject, as compared to the frequency of myoclonic seizures experienced by the subject prior to the administration of the oligomeric compound. In some embodiments, administration of an oligomeric compound to a subject in need thereof in the context of the present disclosure results in a decrease of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75% at least about 80%, at least about 85%, at least about 90% or at least about 95% in the frequency of myoclonic experienced by the subject, as compared to the frequency of myoclonic seizures experienced by the subject prior to the administration of the oligomeric compound.

Interictal epileptiform discharges (IEDs) are abnormal electrical brain activity patterns that occur between seizures in patients with epilepsy (Smith et al., Elife. 2022 Jan. 20; 11:e73541. doi: 10.7554/eLife.73541. PMID: 35050851; PMCID: PMC8813051). It has been reported that IEDs in children with idiopathic epilepsy can affect both cognitive function and academic performance. (Cheng et al., BMC Neurol. 2020 Jun. 6; 20(1):233. doi: 10.1186/s12883-020-01807-z. PMID: 32505173; PMCID: PMC7275426). IEDs can be measured non-invasively using electroencephalography (EEG), a technique that records electrical activity in the brain through electrodes placed on the scalp. This makes it possible to monitor changes in IED frequency in real-time without causing discomfort to the subject.

Without wishing to be bound by a specific theory, it is believed that IEDs may be used clinically, e.g., to test effects of various therapies in subjects with DEEs. For example, IEDs can serve as an objective biomarker for the presence and severity of epileptic activity. By measuring the frequency and distribution in the brain of IEDs before and after administering a drug treatment, the drug's effectiveness in reducing epileptic activity can be monitored. It is also believed that changes in IED frequency and distribution may be detectable before any significant changes in seizure frequency or other clinical signs. Thus, IEDs can provide an early indication of the drug's effectiveness in treating DEE in a clinical trial, and may be used to inform target engagement and dosing strategies. It is also believed that a decrease in IED frequency after drug treatment may be associated with improved seizure control and cognitive outcomes. Thus, monitoring IEDs during a clinical trial can provide valuable information on the potential long-term benefits of the treatment.

In some embodiments, administering an oligomeric compound to a subject in need thereof in the context of the present disclosure results in a decrease in the frequency of IEDs in the subject, as compared to the frequency of IEDs in the subject prior to administering an oligomeric compound. In some embodiments, administering an oligomeric compound to a subject in need thereof in the context of the present disclosure results in a decrease of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75% or at least about 80% in frequency of IEDs in the subject, as compared to the frequency of IEDs in the subject prior to administering an oligomeric compound. In some embodiments, the decrease in the frequency of IEDs in the subject is observed at least 1 week, at least 2 weeks, at least 3 weeks, at least 4 weeks, at least 6 weeks, at least 8 weeks, at least 10 weeks, at least 12 weeks, at least 14 weeks or at least 16 or more weeks after the start of the administration of an oligomeric compound. In some embodiments, administering an oligomeric compound to a subject in need thereof in the context of the present disclosure results in a decrease in the frequency of amplitude-integrated electroencephalography (aEEG) signals, as compared to the frequency of aEEG signals in the subject prior to administering an oligomeric compound. In some embodiments, administering an oligomeric compound to a subject in need thereof in the context of the present disclosure results in a decrease of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75% or at least about 80% in frequency of aEEG signals in the subject, as compared to the frequency of aEEG signals in the subject prior to administering an oligomeric compound. In some embodiments, the decrease in the frequency of aEEG signals in the subject is observed at least 1 week, at least 2 weeks, at least 3 weeks, at least 4 weeks, at least 6 weeks, at least 8 weeks, at least 10 weeks, at least 12 weeks, at least 14 weeks or at least 16 or more weeks after the start of the administration of an oligomeric compound. In other embodiments, the decrease in frequency of aEEG signals in a subject is observed within 24 hours after the start of administration of an oligomeric compound.

### EXEMPLIFICATION OF THE INVENTION

### Example 1: A Seamless, Clinical Trial to Investigate the Safety and Efficacy of Multiple Doses of Compound 1 in Pediatric Participants with Early Onset SCN2A Developmental and Epileptic Encephalopathy

This is a seamless, in part non-randomized, open label, and in part randomized, placebo procedure-controlled, double-blind, clinical trial to explore the safety, tolerability, PK, and efficacy of ascending doses of Compound 1 in pediatric participants with early onset SCN2A DEE, aged 2 to 18 years. The trial is being conducted in 4 parts: the preliminary safety Part 1 (open label), dose-escalation Part A (double blind), confirmatory Part B (double blind), followed by an open-label extension in Part C.

PART 1: PRELIMINARY SAFETY

| | |
|---|---|
| Objective | Endpoint |

Primary

| | |
|---|---|
| To evaluate the safety and tolerability of | Incidence and severity of treatment-emergent |
| Compound 1 administered by intrathecal | adverse events (TEAEs) |
| (IT) injection in participants with early | Changes in findings on physical and |
| onset SCN2A DEE | neurological examinations |
| | Changes in video electroencephalogram |
| | (vEEG) characteristics as determined by a |
| | central reader |
| | Changes in vital sign measurements |
| | Changes in clinical laboratory results |
| | Changes in electrocardiogram (ECG) |
| | parameters |

Secondary

| | |
|---|---|
| To explore the preliminary efficacy of | Seizure frequency per 28 days, as assessed by |
| Compound 1 in participants with early | seizure diary, over the time period after |
| onset SCN2A DEE | the 4^{th} dose administration |
| | Seizure frequency per 28 days over the time |
| | period immediately after each dose |
| | administration |
| | Percent change from baseline in seizure |
| | frequency per 28 days over the time period |
| | immediately after each dose administration |
| | Response, defined as ≥50% reduction in |
| | seizure frequency per 28 days over the time |
| | period immediately after each dose |
| | administration |

PK

| | |
|---|---|
| To characterize the pharmacokinetics (PK) of | Plasma and cerebrospinal fluid (CSF) |
| Compound 1 | concentrations of Compound 1 |
| | Plasma Compound 1 PK parameters |

Exploratory

| | |
|---|---|
| To explore additional preliminary efficacy of | Electroencephalogram (EEG) characteristics, |
| Compound 1 | including but not limited to electrographic |
| | seizures, interictal epileptiform discharges, |
| | background frequency, and features of sleep |
| | at each post dose timepoint |
| To evaluate the impact of Compound 1 on | Levels of inflammatory markers (including |
| inflammatory markers | C-reactive protein [CRP] and complement |
| | [test for total complement activity - CH50, |
| | C3a, Bb and C5a], fibrinogen) |
| To evaluate the potential for immunogenicity | Presence of anti-drug antibodies (ADA) in |
| towards Compound 1 | plasma |

PART A: DOSE ESCALATION

| | |
|---|---|
| Objective | Endpoint |

Primary

| | |
|---|---|
| To evaluate the safety and tolerability of | Incidence and severity of TEAEs |
| ascending doses of Compound 1 | Changes in findings on physical and |
| administered by intrathecal (IT) injection | neurological examinations |
| in participants with early onset SCN2A | Changes in vEEG characteristics as |
| DEE | determined by a central reader |
| | Changes in vital sign measurements |
| | Changes in clinical laboratory results |
| | Changes in ECG parameters |

Secondary

| | |
|---|---|
| To explore the preliminary efficacy of | Seizure frequency per 28 days, as assessed by |
| Compound 1 compared with placebo in | seizure diary, over the time period after |
| participants with early onset SCN2A | the 6^{th} dose administration |
| DEE | Seizure frequency per 28 days over the time |
| | period immediately after each dose |
| | administration |
| | Percent change from baseline in seizure |
| | frequency per 28 days over the time period |
| | immediately after each dose administration |
| | Response, defined as ≥50% reduction in |
| | seizure frequency per 28 days over the time |
| | period immediately after each dose |
| | administration |

PK

| | |
|---|---|
| To characterize the PK of Compound 1 | Plasma and CSF concentrations of Compound |
| | 1 |
| | Plasma Compound 1 PK parameters |

Exploratory

| | |
|---|---|
| To explore additional preliminary efficacy of | EEG characteristics, including but not limited |
| Compound 1 compared with placebo | to electrographic seizures, interictal |
| | epileptiform discharges, background |
| | frequency, and features of sleep at each post |
| | dose timepoint |
| | Clinical Global Impression-Severity (CGI-S) |
| | at baseline and Clinical Global Impression- |
| | Improvement (CGI-I) scores at each post |
| | dose timepoint |
| | Caregiver Global Impression-Severity |
| | (CgGI-S) at baseline and Caregiver Global |
| | Impression-Improvement (CgGI-I) scores at |
| | each post dose timepoint |
| | Quality of life as assessed by Quality of Life |
| | Inventory-Disability (QI-Disability) at the |
| | end of Part A |
| | evelopmental milestones as assessed by |
| | Bayley Scales of Infant Development - |
| | Third Edition (Bayley-3) domain and |
| | subtest scores |
| | Behavior as assessed by Aberrant Behavior |
| | Checklist - 2^{nd} Edition (ABC-2) total and |
| | subscale scores at the end of Part A |
| | Sleep as assessed by Sleep Disturbance Scale |
| | for Children at the end of Part A |
| To evaluate the impact of Compound 1 on | Levels of inflammatory markers (including |
| inflammatory markers | CRP and complement [CH50, C3a, Bb and |
| | C5a], fibrinogen) |
| To evaluate the potential for immunogenicity | Presence of ADA in plasma |
| towards Compound 1 | |

PART B: CONFIRMATORY

| | |
|---|---|
| Objective | Endpoint |

Primary

| | |
|---|---|
| To assess the efficacy of Compound 1 | Seizure frequency per 28 days, as assessed |
| administered by IT injections compared | by seizure diary, over the time period after |
| with placebo in participants with early | the 6^{th} dose administration |
| onset SCN2A DEE | |

Secondary

| | |
|---|---|
| To assess the secondary efficacy of | Seizure frequency per 28 days during the time |
| Compound 1 administered by IT | period immediately after each dose |
| injections compared with placebo in | administration |
| participants with early onset SCN2A DEE | Percent change from baseline in seizure |
| | frequency per 28 days over the time period |
| | immediately after each dose administration |
| | Response, defined as ≥50% reduction in |
| | seizure frequency per 28 days, over the |
| | time period immediately after each dose |
| | administration |
| | EEG characteristics, including but not limited |
| | to electrographic seizures, interictal |
| | epileptiform discharges, background |
| | frequency, and features of sleep at each |
| | post dose timepoint |
| | CGI-S at baseline and CGI-I scores at each |
| | post dose timepoint |
| | CgGI-S at baseline and CgGI-I scores at each |
| | post dose timepoint |
| | Quality of life as assessed by Quality of Life |
| | Inventory-Disability (QI-Disability) at the |
| | end of Part B |
| | Developmental milestones as assessed by |
| | Bayley Scales of Infant Development - |
| | Third Edition (Bayley-3) domain and |
| | subtest scores |
| | Behavior as assessed by ABC-2 total and |
| | subscale scores at the end of Part B |
| | Sleep as assessed by Sleep Disturbance Scale |
| | for Children at the end of Part B |

Safety

| | |
|---|---|
| To evaluate the safety and tolerability of | Incidence and severity of TEAEs |
| Compound 1 administered by IT injection | Changes in findings on physical and |
| in participants with early onset SCN2A | neurological examinations |
| DEE | Changes in vEEG characteristics as |
| | determined by a central reader |
| | Changes in vital sign measurements |
| | Changes in clinical laboratory results |
| | Changes in ECG parameters |

PK

| | |
|---|---|
| To characterize the PK of Compound 1 | Plasma and CSF concentrations of Compound |
| | 1 |
| | Plasma Compound 1 PK parameters |

Exploratory

| | |
|---|---|
| To evaluate the impact of Compound 1 on | Levels of inflammatory markers (including |
| inflammatory markers | CRP and complement [CH50, C3a, Bb and |
| | C5a], fibrinogen) |
| To evaluate the potential for | Presence of ADA in plasma |
| immunogenicity towards Compound 1 | |

PART C: OPEN-LABEL EXTENSION

| | |
|---|---|
| Objective | Endpoint |

Exploratory

| | |
|---|---|
| To evaluate the durability of efficacy of | Seizure frequency per 28 days, as assessed |
| Compound 1 administered by IT | by seizure diary, over the time period |
| injections at a maintenance dosing | immediately after each dose administration |
| schedule | Percent change from baseline in seizure |
| | frequency per 28 days over the time period |
| | immediately after each dose administration |
| | Response, defined as ≥50% reduction in |
| | seizure frequency per 28 days over the time |
| | period after each dose administration |
| | EEG characteristics, including but not |
| | limited to electrographic seizures, interictal |
| | epileptiform discharges, background |
| | frequency, and features of sleep at each |
| | post dose timepoint |
| | CGI-S at baseline and CGI-I scores at each |
| | post dose timepoint |
| | CgGI-S at baseline and CgGI-I scores at each |
| | post dose timepoint |
| | Quality of life as assessed by Quality of Life |
| | Inventory-Disability (QI-Disability) at the |
| | end of Part C |
| | Developmental milestones as assessed by |
| | Bayley Scales of Infant Development - |
| | Third Edition (Bayley-3) domain and |
| | subtest scores |
| | Behavior as assessed by ABC-2 total and |
| | subscale scores at the end of Part C |
| | Sleep as assessed by Sleep Disturbance Scale |
| | for Children at the end of Part C |
| To evaluate the impact of Compound 1 on | Levels of inflammatory markers (including |
| inflammatory markers | CRP and complement [CH50, C3a, Bb and |
| | C5a], fibrinogen) |
| To evaluate the potential for | Presence of ADA in plasma |
| immunogenicity towards Compound 1 | |

Safety

| | |
|---|---|
| To evaluate the safety and tolerability of | Incidence and severity of TEAEs |
| Compound 1 administered by IT injection | Changes in findings on physical and |
| during maintenance dosing in participants | neurological examinations |
| with early onset SCN2A DEE | Changes in vEEG characteristics as |
| | determined by a central reader |
| | Changes in vital sign measurements |
| | Changes in clinical laboratory results |
| | Changes in ECG parameters |

PK

| | |
|---|---|
| To characterize the PK of Compound 1 | Plasma and CSF concentrations of Compound |
| during maintenance dosing | 1 |
| | Plasma Compound 1 PK parameters |

### Part 1: Preliminary Safety

Part 1 is being conducted with the objective of obtaining clinical safety data which will further inform the dose escalation in Part A. In this open label, non-randomized part of the trial, at least 4 participants are enrolled and are receiving Compound 1 at 1 mg doses at ≥4-week intervals for up to 13 weeks.

Safety, along with available PK data, is being assessed by the sponsor on an ongoing basis along with scheduled quarterly Data Monitoring Committee (DMC) reviews after each interim analysis, and ad hoc reviews as needed based on emerging safety data. Preliminary efficacy in seizure reduction and safety was assessed after 4 doses.

Following their final dose, participants from Part 1 will have the potential to transition to long term extension, subject to the FDA's further review and approval of the trial continuation, and pending updated chronic toxicology data.

### Part A: Dose Escalation

Part A will initiate upon completion of Part 1. The goal of Part A is to identify a cumulative dose of Compound 1 with a favorable benefit-risk profile for seizure reduction that can be further assessed in Part B. Part A will consist of up to 16 participants. Initially, 8 treatment-naïve participants will be randomized 3:1 to receive ascending doses of Compound 1 or a placebo procedure. The first 4 participants must reach at least 4 weeks after their first dose before additional participants can be administered study drug. Administrations will occur at ≥4-week intervals for the first 4 doses, and at ≥6-week intervals for the subsequent doses. Dose escalations will be within-participant. The starting dose will be 1 mg with ≤2-fold increase for subsequent doses with specific dose escalation criteria. The target is to reach a cumulative dose of up to 42 mg, which is predicted to correspond to approximately 35% knock down (KD), divided into 6 administrations.

Safety, along with available PK data, will be assessed by the sponsor on an ongoing basis along with scheduled quarterly DMC reviews and ad hoc reviews as needed based on emerging safety data. Preliminary efficacy in seizure reduction and safety will be assessed during the time period after the 6^{th} dose administration after up to 8 participants have reached a cumulative dose of up to 42 mg or a corresponding placebo procedure.

The dosing regimen may be revised based on the analysis of safety and efficacy. Maximum tolerated dose (MTD) in this trial is defined as 1 dose level below a single or cumulative dose associated with 2 or more Dose Limiting Toxicities (DLTs) in Part A or a 25% rate trial-wide. Up to 8 additional participants may be added (Group 2, randomized to active or placebo in a 3:1 ratio) to further assess the safety and efficacy of escalating doses of Compound 1. The starting dose for these additional participants will be up to the highest tolerated single dose previously administered. A second planned analysis assessing safety and efficacy during the time period after the 6^{th} dose will occur after up to 8 participants have reached up to 100 mg cumulatively. After the final dose (or placebo procedure), participants will subsequently have the opportunity to enter Part C/open-label extension.

### Part B: Confirmatory Phase

Part B will further assess and confirm the safety and efficacy of the cumulative dose identified in Part A in a randomized, placebo procedure-controlled design. Up to 40 treatment-naïve participants will be randomized, with no more than 75% to receive Compound 1 over placebo treatment (3:1 randomization maximum). The final sample size and randomization scheme will be further confirmed based on the preliminary data and power calculations from Part A.

The cumulative dose and highest single dose will not exceed the respective MTD in Part A. The cumulative dose from Part A will be divided into ≤6 administrations of Compound 1 or placebo procedure and will be given no more frequently than every 6 weeks. If tolerability issues arise, participants may be titrated to safely reach the cumulative dose identified in Part A.

The primary endpoint will be seizure frequency over the time period after the 6^{th} dose as assessed by the seizure diary. Thus, the duration of participation in Part B is up to 30 weeks. After the final dose (or placebo procedure) participants will subsequently have the opportunity to enter the open-label extension of the trial, Part C.

Parts 1, A, and B will each consist of 3 periods: Screening Period, Intervention Period (open-label in Part 1, double-blind in Parts A and B), and Follow-up Period.

### Screening Period

Prior to any clinical trial procedures, the participant/caregiver will provide written informed consent and satisfy inclusion/exclusion criteria. Key assessments during Screening will include medical and disease history, demographic data, body weight, height and head circumference, physical examination (including detailed neurological examination), clinical laboratory evaluations, magnetic resonance imaging (MRI) of the brain, vital signs measurements, 12-lead ECGs, and a review of concomitant medications/procedures.

Caregivers will complete a daily seizure diary for at minimum 4 weeks during the Baseline Observation period (prior to Baseline Dosing Visit) through EOT, including the days without visits. Other screening assessments may still occur during this 4-week period (and do not necessarily have to be completed prior to the start of the daily seizure diary). Each participant's eligibility will be reviewed and approved by an Eligibility Review Committee (ERC) before they are included into the trial. A baseline prolonged vEEG will be completed within the 4 weeks prior to Baseline Dosing Visit.

### Intervention Period

Part 1 (Open-label Intervention): Participants will be admitted to the trial site on Day −1 for Baseline Dosing Visit. On Day 1, after confirmation of eligibility, they will receive Compound 1 via IT administration. Each participant will remain inpatient for a minimum of 24 hours, and up to 48 hours after dosing for physical examination (including detailed neurological examination), clinical laboratory evaluations, vital signs measurements, 12-lead ECGs, and any other trial assessments in this visit. The corresponding procedures and assessments including Compound 1 administration, will take place again in intervals of approximately (but not more frequent than) every 4 weeks, for up to 4 doses in total. Dosing in Part 1 may be less frequent based on the tolerability of the study drug.

Approximately 2 weeks after each Compound 1 administration, home-health visits, as well as home vEEG, will be conducted. Home-health visits may be conducted at home (i.e., remote to the investigational site) or in the clinic (at the investigational site), at the discretion of the participant together with the investigator. If there are any concerning AEs, or changes in the participant's examination, the participant may return at the discretion of the investigator for an unscheduled visit in the clinic for further evaluation.

Parts A and B (Double-blind Interventions): Participants will be admitted to the trial site on Day −1 for Baseline Dosing Visit. On Day 1, after confirmation of eligibility, they will receive Compound 1 via IT administration, or placebo procedure per the randomization schedule. Each participant will remain inpatient for a minimum of 24 hours, and up to 48 hours after dosing for physical examination (including detailed neurological examination), clinical laboratory evaluations, vital signs measurements, 12-lead ECGs, and any other trial assessments in this visit. The corresponding procedures and assessments including Compound 1 administration, or placebo procedure, will take place again in intervals of approximately (but not more frequent than) every 4 weeks for the first 4 doses and every 6 weeks for the subsequent doses in Part A, and every 6 weeks in Part B.

Approximately 2 weeks after each Compound 1 administration or placebo procedure, home-health visits, as well as home vEEG, will be conducted. Home-health visits may be conducted at home (i.e., remote to the investigational site) or in the clinic (at the investigational site), at the discretion of the participant together with the investigator. If there are any concerning AEs, or changes in the participant's examination, the participant may return at the discretion of the investigator for an unscheduled visit in the clinic for further evaluation.

### Safety Follow-Up Period

During the 6-month follow-up period, home-health visits, as well as home vEEG, will be conducted. Home-health visits may be conducted at home (i.e., remote to the investigational site) or in the clinic (at the investigational site), at the discretion of the participant together with the investigator. If there are any concerning AEs or changes in the participant's examination (per caregiver report or noted at the home-health visit), the participant may return at the discretion of the investigator for an unscheduled visit in the clinic for further evaluation. A final visit to the clinic will occur for the end of trial assessments.

The Follow-up Period may be extended based on the results of the assessments at the end of study (EOS) visit and the investigator's best clinical judgement. Should it be considered necessary, the extended follow-up will include assessments for the presence of potential long-term and developmental effects.

### Part C: Open-Label Extension

Participants Part C is an open-label extension of the trial that will assess the safety and durability of effect of Compound 1 on seizures and other outcomes measures of a maintenance dosing regimen over up to 2 years. The open label extension will be available for any patient with prior exposure to Compound 1 or if they are rolling over from an active Compound 1 trial.

### Following Part 1

Following their final dose in Part 1, participants will have the potential to transition to the long-term extension in Part C, which will start ≥6 weeks after the final dose in Part 1, and after the FDA approval for dosing in Part C to proceed, whichever occurs later. Part C will have an initial titration period to allow for the maintenance dose to be reached safely. The starting dose in the titration period will be no more than the MTD in Part A, and the exact titration regimen will be determined following DMC's review of the interim analysis results from Part A. During the titration period, participants will receive 4 doses of Compound 1 at 4-week intervals, with the subsequent maintenance doses administered at ≥12-week intervals. All the doses in Part C for participants transitioning from Part 1 will be administered in an unblinded fashion.

If the results of the interim analysis from Part A do not become available within 6 weeks of the Part 1 completion and/or the FDA approval to proceed to Part C is not received within the same timeframe, participants may continue to be dosed with 1 mg of Compound 1, as an early Part C, at ≥6-week intervals in the interim, before commencing the titration period in Part C. This interim dosing may start ≥6 weeks after the final dose in Part 1 and continue until both the FDA approval for dosing in Part C is received and the titration regimen for this part is determined based on the results of the interim analysis from Part A.

### Following Part A or B

Participants from Parts A and B will have the potential to enroll in Part C. Part C will have an initial titration period to allow participants which previously received placebo in Parts A or B to reach the Compound 1 maintenance dose safely, and to avoid breaking the study blind. During the titration period, participants who were on placebo in Part A or B will be administered 4 ascending doses of Compound 1 at 4-week intervals, with the fourth titration dose planned to be the MTD from Parts A and B. Participants who were on active in Part A or B will be administered the MTD of Compound 1 at the start (dose 1) and at the end of the titration period (dose 4); doses 2 and 3 of the titration regimen for these participants will comprise the placebo procedure. After the initial titration period, all the participants will receive Compound 1 maintenance doses at 12-week intervals. The first 3 titration doses in Part C for participants transitioning from Part A or B will be administered blinded; all other doses in Part C will be administered in an unblinded fashion.

The highest dose administered in Part C will be up to the maximum tolerated single dose from Part A and Part B. This dose level may change as Part A and Part B progress. It is expected that dosing every 12 weeks will be sufficient to maintain KD levels. Therefore, following the titration period, dosing will be at least 12 weeks apart for up to a total of 2 years. Participants may receive lower doses or less frequent dosing based on their tolerability.

The Follow-up Period may be extended based on the results of the assessments at the EOS visit and the investigator's best clinical judgement. Should it be considered necessary, the extended follow-up will include assessments for the presence of potential long-term and developmental effects.

### Efficacy Assessments

### Seizure Diary

An electronic device (e.g., tablet/phone, watch application, and/or other devices) was used to collect information about participant's seizures and daily concomitant medication use during the clinical trial. The diary was completed daily for at minimum 28 days prior to Baseline dosing visit, during the Baseline Observation Period (after the Screening visit) and daily throughout the remainder of the clinical trial. The diary from the Baseline Observation Period serves as an eligibility check, and to establish a baseline. Prompts were used to ensure adequate data capture in addition to spontaneous reporting. The diary was completed by the same caregiver as often as possible. Daily seizure diary data collected as part of an ongoing observation trial that falls within the Screening window per this protocol may be used without having to be repeated.

### Video EEG

To the extent allowed by local regulations, a vEEG was performed by a trained technician throughout the trial in order to record brainwave activity and to evaluate for changes over time. Prolonged vEEG was completed at home or in a facility, ideally with a minimum of 12 hours of continuous recording and capturing periods of both wakefulness and sleep. vEEGs collected within the Screening window per this protocol, as part of an ongoing observational trial, would not have to be repeated during the Screening period.

### Bayley Scales of Infant Development-Third Edition (Bayley-3)

The Bayley-3 is a standardized neurodevelopmental assessment measure used by clinicians to evaluate key domains in early childhood development for individuals between 16 days and 42 months after birth. These domains include adaptive behavior, cognition, language, motor function (gross and fine), and social-emotional development. The Bayley-3 assessments will be video recorded to the extent allowed by local regulations.

### Sleep Disturbance Scale for Children

The parent-reported Sleep Disturbance Scale for Children (SDSC) is a 27-item scale rated on a 5-point Likert scale and designed to categorize sleep disorders in children. In addition to an overall score, the instrument provides 5 sub-scores for the following: disorders of initiating and maintaining sleep, sleep breathing disorders, disorders of arousal or sleep-wake transition disorders, disorders of excessive somnolence, and sleep hyperhidrosis.

### Quality of Life Inventory-Disability (QI-Disability)

The QI-Disability is a parent-report measure for children with intellectual disabilities. It is a reliable and valid measure of the quality of life across the spectrum of intellectual disability. It has the potential to allow for clearer identification of support needs and measures responsiveness to interventions.

### Aberrant Behaviors Checklist-2^{nd} Edition (ABC-2)

The ABC-2 is a clinician-assessed rating scale that measures the severity of a range of problem behaviors commonly observed in individuals with intellectual disabilities.

### Clinical Global Impression-Severity (CGI-S) and Clinical Global Impression-Improvement (CGI-I)

The CGI was developed for use in NIH-sponsored clinical trials in individuals with mental health disorders. The CGI provides an overall assessment of improvement over a specified period. The CGI includes two, 7-point Likert rating scales: the CGI-S and the CGI-I scale. The CGI will be anchored with reference to domains/symptoms experienced by participants with SCN2A-DEE. Participants will be assessed by the clinician at Baseline (Day 1) for the severity of SCN2A-DEE symptoms using the CGI-S. Change from baseline in SCN2A-DEE symptoms will be assessed by the clinician using the CGI-I.

### Caregiver Global Impression-Severity (CgGI-S) and Caregiver Global Impression-Improvement (CgGI-I)

These scales are similar to the CGI-S and CGI-I. Participants will be assessed by the caregiver at Baseline (Day 1) for the severity of their SCN2A-DEE symptoms using the CgGI-S. Change from baseline in SCN2A-DEE symptoms will be assessed by the caregiver using the CgGI-I.

Results from Part 1: Preliminary Safety

In part 1, participants received Compound 1 intrathecally at ≥4 week intervals for up to 13 weeks. The incidence and severity of treatment-emergent adverse events was assessed, with preliminary efficacy and safety also assessed after 4 doses.

Four subjects participated in Part 1 of the trial as shown in Table 1 below.

TABLE 1
Subject Demographics

| | Age at | | | | Number of |
|---|---|---|---|---|---|
| Subject | consent | | | | dosing visits |
| ID | (years) | Gender | Race | Ethnicity | completed |
| 2001 | 3 | Female | White | Not Hispanic | 4 |
| | | | | or Latino | |
| 2002 | 14 | Male | White | Not Hispanic | 4 |
| | | | | or Latino | |
| 2003 | 2 | Female | White | Not Hispanic | 4 |
| | | | | or Latino | |
| 2004 | 2 | Male | Other | Hispanic or | 4 |
| | | | (Hispanic) | Latino | |

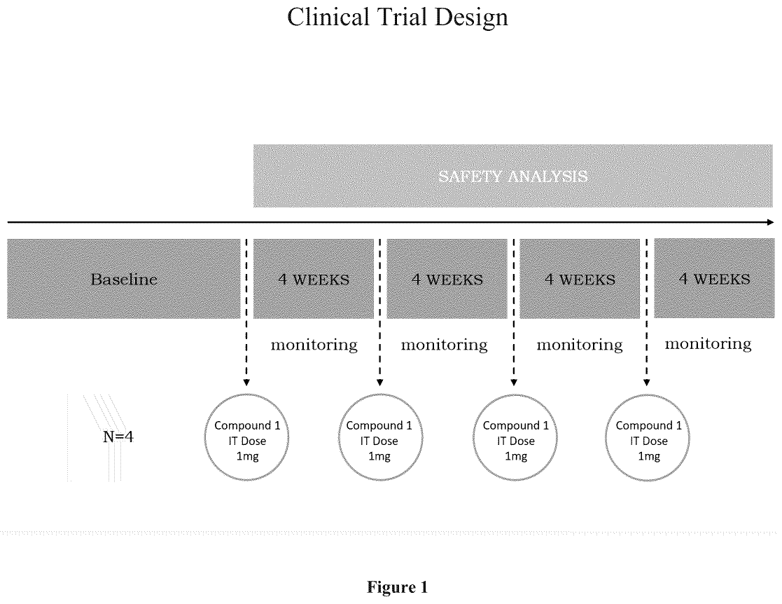

Each subject received 1 mg of Compound 1 every 4 weeks, or monthly. The schematic illustrating dosing of the subjects is shown in FIG. 1.

The subjects exhibited no clinically significant abnormalities in vital signs and physical and neurological signs upon examination.

Table 2 below shows the summary values of the seizure diary data for the subjects.

TABLE 2
Summary values of the seizure diary data

| | | | | % reduction in the | |
|---|---|---|---|---|---|
| | | | | number of seizures | |
| | # of seizures | | | from baseline | |
| Subject | | Dose | Dose | After | After |
| ID | Baseline | Period 1 | Period 2 | 1^{st} Dose | 2^{nd} dose |
| 2001 | 146 | 56 | 84 | 61.64 | 42.46 |
| 2002 | 41 | 21 | 22 | 48.78 | 46.34 |
| 2003 | 10 | 0 | 7 | 100 | 30 |
| 2004 | 345 | 331 | 134 | 4.05 | 61.16 |
| | | | | 1^{st} Dose | 2^{nd} Dose |
| | | | % mean reduction | 53.61 | 44.98 |
| | | | in the number | | |
| | | | of seizures | | |
| | | | % median | 55.21 | 44.00 |
| | | | reduction in | | |
| | | | the number | | |
| | | | of seizures | | |

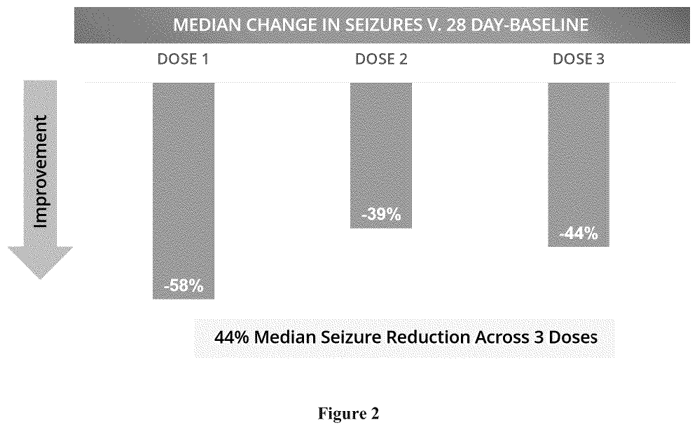

FIG. 2 is a bar graph showing median change in seizures vs. 28 day-baseline after 3 doses. The results shown in FIG. 2 indicate that Compound 1 exhibits efficacy in reducing seizures even after 1 dose.

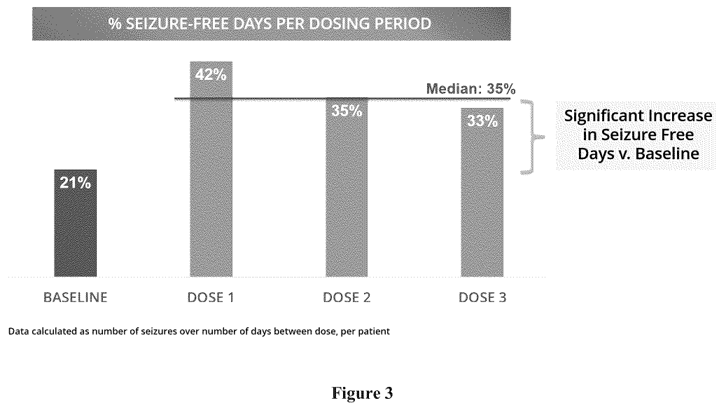

FIG. 3 is a bar graph showing % of seizure free day per dosing period, calculated as number of seizures over number of days between dose, per patient, after 3 doses. The results shown in FIG. 3 indicate that there is a significant increase in seizure-free days vs. baseline, with improvements observed after one dose.

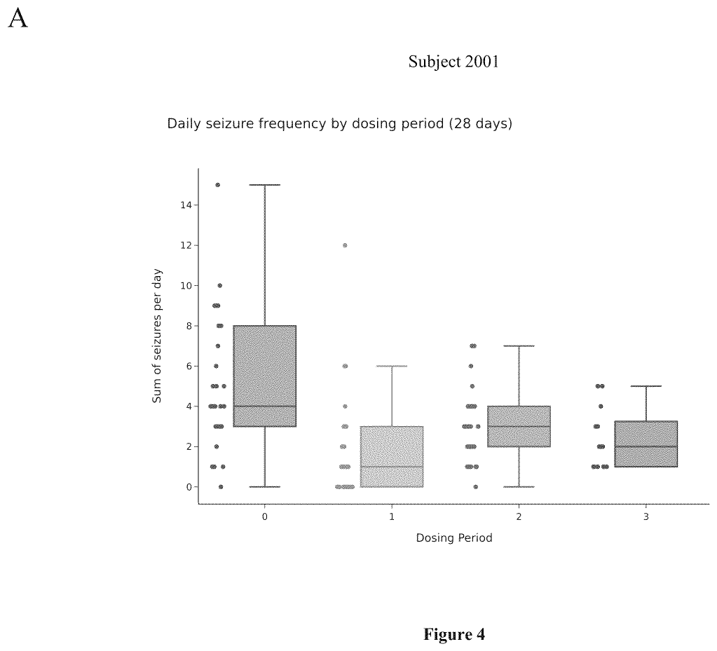

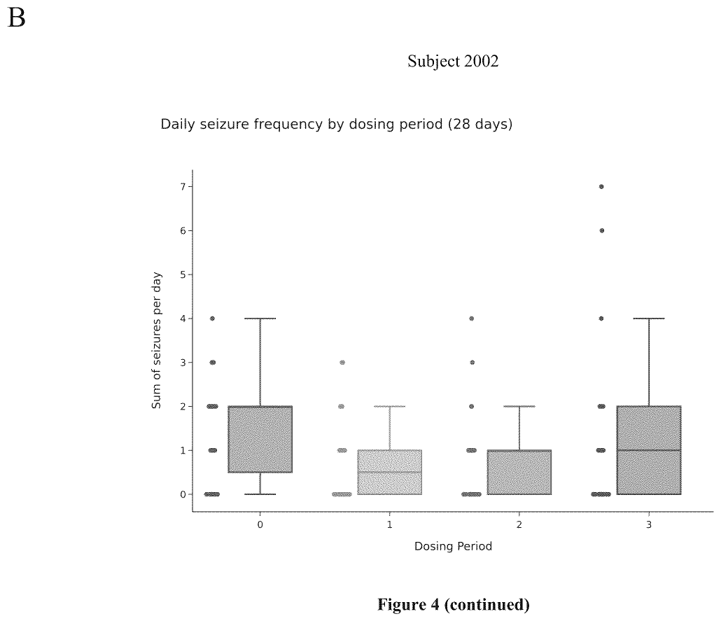

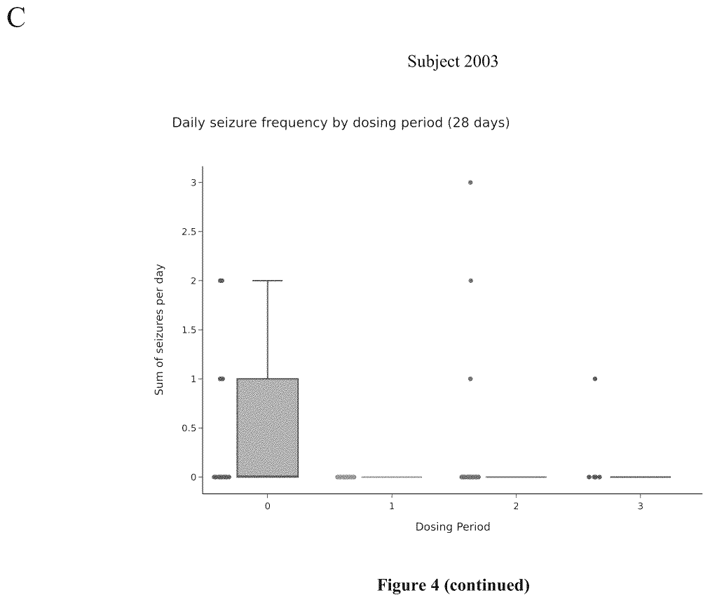

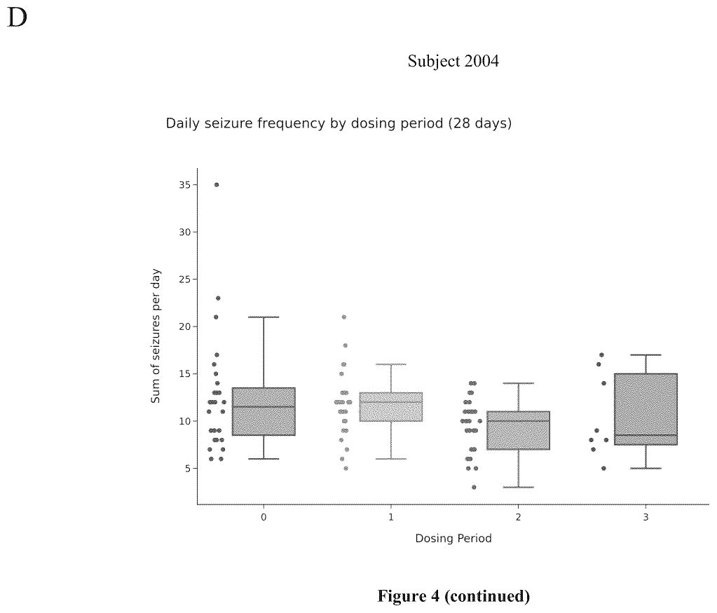

FIG. 4, Panel A is a boxplot showing daily seizure frequency by dosing period (28 days) for subject 2001 after 3 doses.

FIG. 4, Panel B is a boxplot showing daily seizure frequency by dosing period (28 days) for subject 2002 after 3 doses.

FIG. 4, Panel C is a boxplot showing daily seizure frequency by dosing period (28 days) for subject 2003 after 3 doses.

FIG. 4, Panel D is a boxplot showing daily seizure frequency by dosing period (28 days) for subject 2004 after 3 doses.

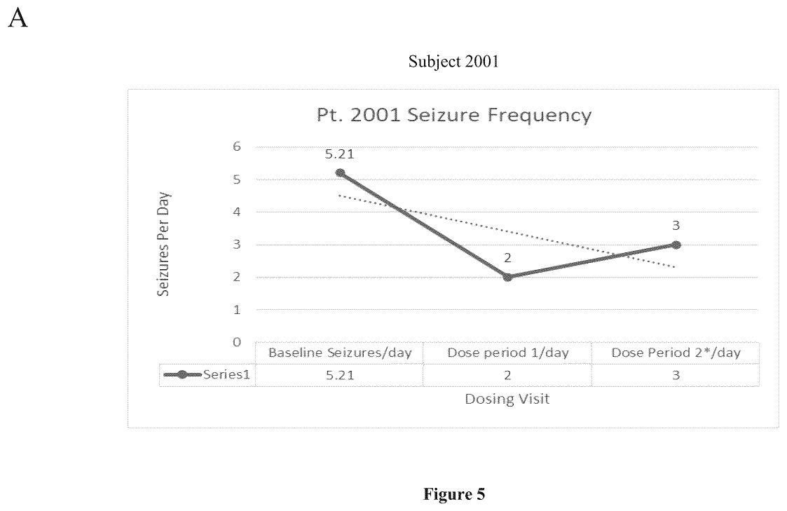

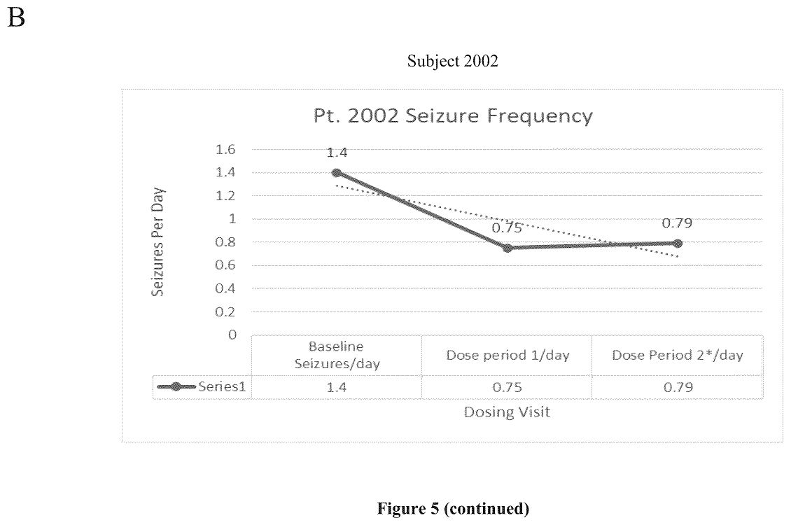

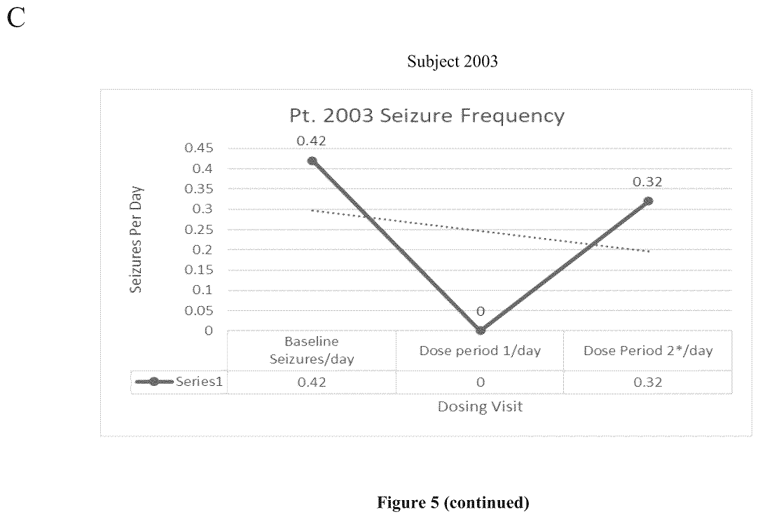

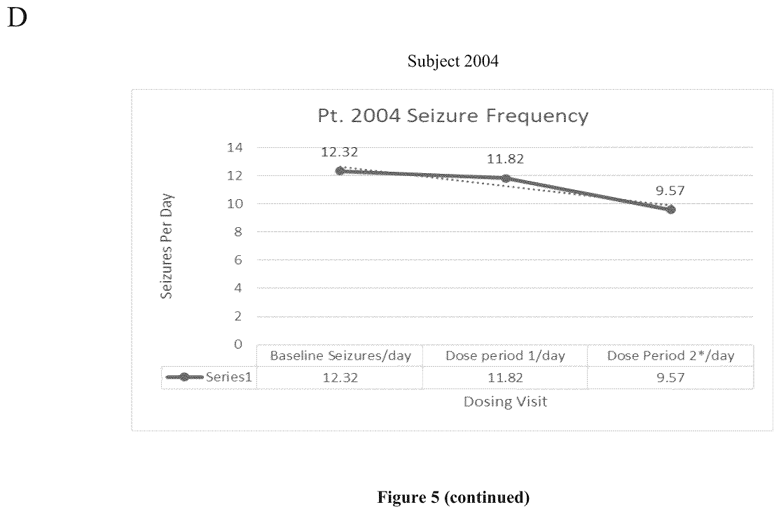

FIG. 5, Panel A is a graph showing daily seizure frequency per dose period for subject 2001 after 3 doses.

FIG. 5, Panel B is a graph showing daily seizure frequency per dose period for subject 2002 after 3 doses.

FIG. 5, Panel C is a graph showing daily seizure frequency per dose period for subject 2003 after 3 doses.

FIG. 5, Panel D is a graph showing daily seizure frequency per dose period for subject 2004 after 3 doses.

Table 3 below shows seizure diary summary data for Subject 2001 after 3 doses.

TABLE 3
Seizure summary data for subject 2001.

| | | Dosing Period | | |
|---|---|---|---|---|
| | Baseline | 1 | 2 | 3 |
| Number of days in the dosing | 28 | 28 | 28 | 13 |
| period | | | | |
| Average daily seizures | 5.21 | 2.0 | 3 | 2.38 |
| Number of seizure free days | 1 | 9 | 1 | 0 |
| Number of focal motor seizures | 43 | 31 | 25 | 11 |
| Number of tonic seizures | 50 | 10 | 34 | 12 |
| Number of generalized tonic- | 0 | 0 | 0 | 0 |
| clonic seizures | | | | |
| Number of myoclonic seizures | 50 | 6 | 19 | 8 |
| Number of times rescue | 5 | 5 | 12 | 4 |
| medication was used | | | | |
| Average percentage rescue | 17.86% | 17.86% | 42.86% | 30.77% |
| medication | | | | |

Table 4 below shows seizure type frequency data for subject 2001.

TABLE 4
Seizure type frequency data for subject 2001 after 2 doses.

| | Baseline | Dosing Period | | % reduction after | |
|---|---|---|---|---|---|
| | Seizures/month | 1 | 2 | 1 dose | 2 doses |
| Focal | 43 | 31 | 25 | 27.90 | 41.86 |
| seizures | | | | | |
| Tonic | 50 | 10 | 34 | 80 | 32 |
| GTC | No generalized | No generalized | No generalized | No generalized | No generalized |
| | T-C seizures | T-C seizures | T-C seizures | T-C seizures | T-C seizures |
| | in the period | in the period | in the period | in the period | in the period |
| Myoclonic | 50 | 6 | 19 | 88 | 62 |

Table 5 below shows seizure diary summary data for Subject 2002 after 3 doses.

TABLE 5
Seizure summary data for subject 2002.

| | | Dosing Period | | |
|---|---|---|---|---|
| | Baseline | 1 | 2 | 3 |
| Number of days in the dosing period | 28 | 28 | 28 | 8 |
| Average daily seizures | 1.46 | 0.75 | 0.79 | 0.25 |
| Number of seizure free days | 7 | 14 | 13 | 7 |
| Number of focal motor seizures | 0 | 0 | 1 | 0 |
| Number of tonic seizures | 40 | 21 | 21 | 2 |
| Number of generalized tonic-clonic | 0 | 0 | 0 | 0 |
| seizures | | | | |
| Number of myoclonic seizures | 0 | 0 | 0 | 0 |
| Number of times rescue medication | 0 | 0 | 0 | 0 |
| was used | | | | |
| Average percentage rescue medication | 0% | 0% | 0% | 0% |

Table 6 below shows seizure type frequency data for subject 2002.

TABLE 6
Seizure type frequency data for subject 2002 after 2 doses.

| | Baseline | Dosing Period | | % reduction after | |
|---|---|---|---|---|---|
| | Seizures/month | 1 | 2 | 1 dose | 2 doses |
| Focal | No focal | No focal | 1 | No focal | Increase |
| seizures | seizures in | seizures | | seizures | of 1% |
| | the baseline | | | | |
| Tonic | 40 | 21 | 21 | 47.5 | 47.5 |
| GTC | No generalized | No generalized | No generalized | No generalized | No generalized |
| | T-C seizures | T-C seizures | T-C seizures | T-C seizures | T-C seizures |
| | in the period | in the period | in the period | in the period | in the period |
| Myoclonic | No myoclonic | No myoclonic | No myoclonic | No myoclonic | No myoclonic |
| | seizures in the | seizures in | seizures in | seizures in | seizures in |
| | baseline period | the period | the period | the period | the period |

Table 7 below shows seizure diary summary data for Subject 2003 after 3 doses.

TABLE 7
Seizure summary data for subject 2003.

| | | Dosing Period | | |
|---|---|---|---|---|
| | Baseline | 1 | 2 | 3 |
| Number of days in the dosing period | 28 | 28 | 28 | |
| Average daily seizures | 0.42 | 0 | 0.25 | |
| Number of seizure free days | 17 | 23 | 24 | |
| Number of focal motor seizures | 0 | 0 | 0 | |
| Number of tonic seizures | 9 | 0 | 4 | |
| Number of generalized tonic-clonic | 0 | 0 | 0 | |
| seizures | | | | |
| Number of myoclonic seizures | 0 | 0 | 0 | |
| Number of times rescue medication | 2 | 0 | 1 | |
| was used | | | | |
| Average percentage rescue medication | 4.17% | 0% | 3.57% | |

Table 8 below shows seizure type frequency data for subject 2003.

TABLE 8
Seizure type frequency data for subject 2003 after 2 doses.

| | Baseline | Dosing Period | | % reduction after | |
|---|---|---|---|---|---|
| | Seizures/month | 1 | 2 | 1 dose | 2 doses |
| Focal | No focal | No focal | No focal | No focal | No focal |
| seizures | seizures in | seizures in | seizures in | seizures in | seizures in |
| | the baseline | the period | the period | the period | the period |
| Tonic | 9 | 0 | 4 | 100 | 55.56 |
| GTC | No generalized | No generalized | No generalized | No generalized | No generalized |
| | T-C seizures | T-C seizures | T-C seizures | T-C seizures | T-C seizures |
| | in the period | in the period | in the period | in the period | in the period |
| Myoclonic | No myoclonic | No myoclonic | No myoclonic | No myoclonic | No myoclonic |
| | seizures in the | seizures in | seizures in | seizures in | seizures in |
| | baseline period | the period | the period | the period | the period |

Table 9 below shows seizure diary summary data for Subject 2004.

TABLE 9
Seizure summary data for subject 2004 after 3 doses.

| | | Dosing Period | | |
|---|---|---|---|---|
| | Baseline | 1 | 2 | 3 |
| Number of days in the dosing | 28 | 28 | 21 | |
| period | | | | |
| Average daily seizures | 12.32 | 11.82 | 9.81 | |
| Number of seizure free days | 0 | 0 | 0 | |
| Number of focal motor seizures | 0 | 0 | 0 | |
| Number of tonic seizures | 325 | 277 | 167 | |
| Number of generalized tonic- | 20 | 54 | 39 | |
| clonic seizures | | | | |
| Number of myoclonic seizures | 0 | 0 | 0 | |
| Number of times rescue | 25 | 3 | 12 | |
| medication was used | | | | |
| Average percentage rescue | 17.86% | 10.71% | 9.52% | |
| medication | | | | |

Table 10 below shows seizure type frequency data for subject 2004.

TABLE 10
Seizure type frequency data for subject 2004 after 2 doses.

| | Baseline | Dosing Period | | % reduction after | |
|---|---|---|---|---|---|
| | Seizures/month | 1 | 2 | 1 dose | 2 doses |
| Focal | No focal | No focal | No focal | No focal | No focal |
| seizures | seizures in | seizures in | seizures in | seizures in | seizures in |
| | the baseline | he period | the period | the period | the period |
| Tonic | 325 | 277 | 109 | 14.76 | 66.46 |
| GTC | 20 | 54 | 25 | −170 | −25 |
| | | | | (increase) | (increase) |
| Myoclonic | No myoclonic | No myoclonic | No myoclonic | No myoclonic | No myoclonic |
| | seizures in the | seizures in | seizures in | seizures in | seizures in |
| | baseline period | the period | the period | the period | the period |

A vEEG scores were determined for each subject at base line and after each dosing period. In the vEEG scoring system, normal vEEG score=0, and a pathological vEEG score is greater than 0, with the worst score being 11. No changes in the vEEG scores throughout the dosing periods were observed for subject 2004, and an increase in the vEEG scores was observed for subjects 2001, 2002 and 2003 as shown in Table 11 below.

TABLE 11
VEEG scores for subjects during each dosing period after 2 doses.

Dosing Period

| Subject ID | Baseline | 1 | 2 |
|---|---|---|---|
| 2001 | 7.8 | 11 | 9 |
| 2002 | 1.8 | 5.4 | 7.6 |
| 2003 | 2.2 | 7.6 | 7.6 |
| 2004 | 11 | 11 | 11 |

It is noted that there are limitations of the vEEG scoring system. Specifically, vEEG scores are highly impacted by numerous variables and represent a snapshot of one day. The vEEG scoring system is not yet validated, so caution is needed when interpreting the results.

During the study, subjects 2001 and 2004 experienced serious adverse events that were unrelated to the study drug. Subjects 2001, 2002 and 2004 also experienced non serious adverse events that were mild or moderate in severity. All TEAEs were resolved.

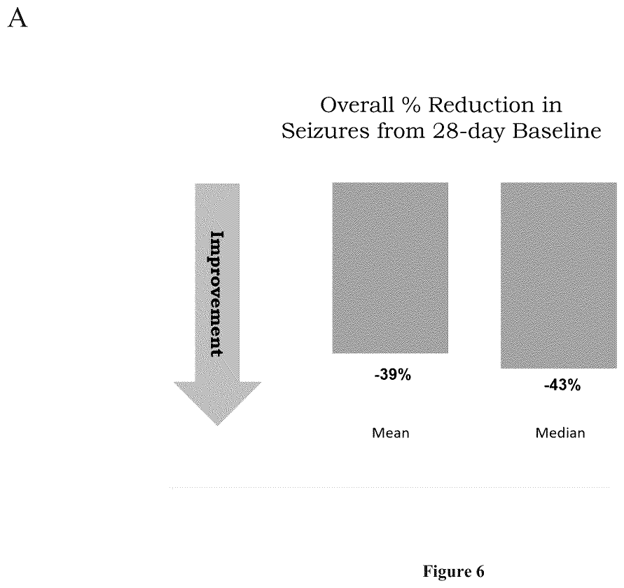

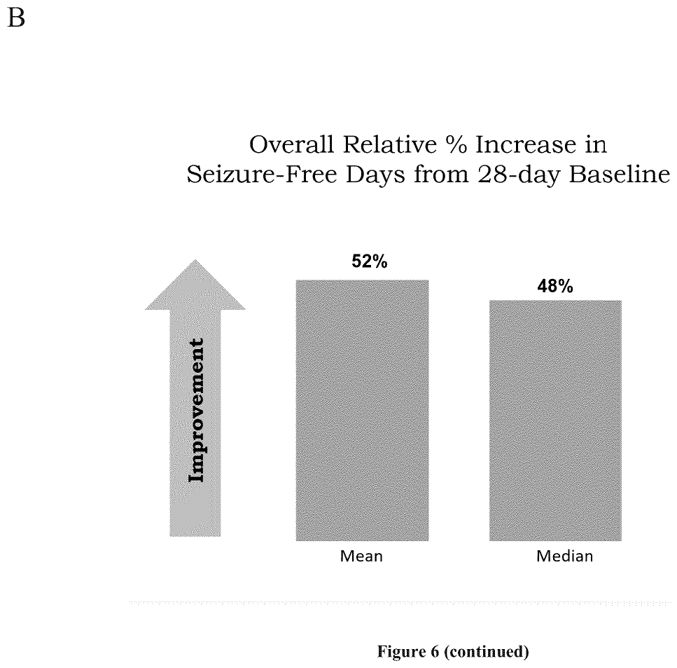

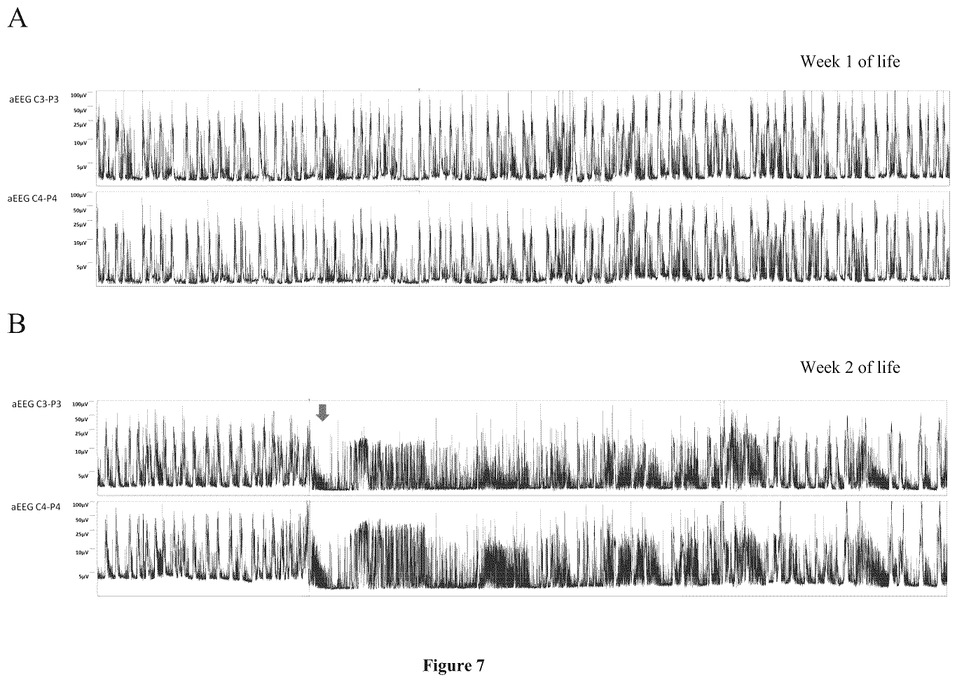

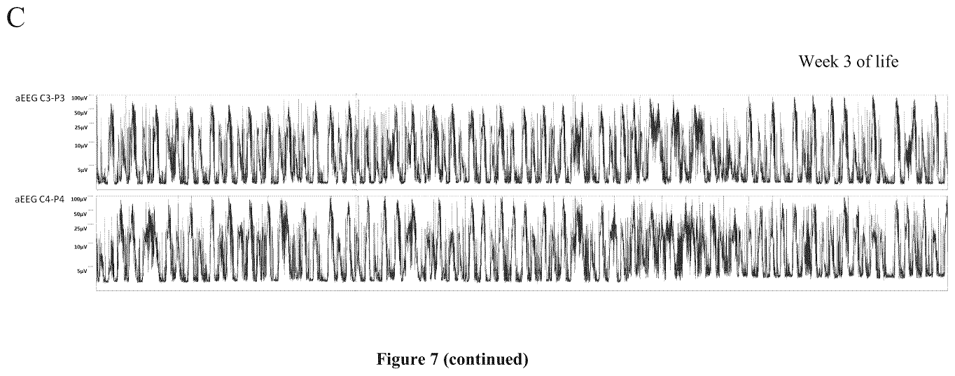

FIG. 6, Panel A is a bar graph showing mean and median change from baseline in seizure frequency for four subjects. The results represent overall percentage reduction from baseline observed through four 28-day periods for 4 subjects.

FIG. 6, Panel B is a bar graph showing mean and median relative percentage change from baseline in seizure-free days for 4 subjects. The results represent overall relative percentage increase in proportion of seizure-free days for 4 subjects.

Safety assessment after 4 doses showed that Compound 1 is well-tolerated with no drug-related AEs. Specifically, no TEAEs or SAEs were related to Compound 1, and all TEAEs were recovered or resolved. Assessments, such as physical and neurological examinations, vital sign measurements and electrocardiogram (ECG) parameters showed no clinically significant changes. Clinical laboratory results also showed no clinically significant changes, except for “elevated WBC” reported for 1 subject. There were 3 subjects with any TEAEs, of which 3 subjects had non-serious TEAEs and 2 subjects had any serious TEAEs. There were 10 individual TEAEs, of which 5 were non-serious TEAEs and 5 were any serious TEAEs. The any serious TEAEs included infection, which is common in this subject population.

Conclusions from Part 1

The preliminary results demonstrate that Compound 1 achieved significant and sustained seizure reduction at 1 mg dose levels, with unexpected benefits across all treated participants. Compound 1 was also well-tolerated with no drug-related AEs. Thus, preliminary results demonstrate tolerability of Compound 1 and its unprecedented efficacy in early onset SCN2A-DEE.

After administration of 4 doses of Compound 1, participants achieved a 43% median reduction in seizures from baseline on top of best available standard of care, and had an increased number of days without seizures, achieving a 48% relative median increase in seizure-free days from baseline. Treated participants were 1.6 times more likely to experience a seizure-free day. There were no TEAEs or SAEs considered related to study drug, and all TEAEs recovered/resolved.

Compound 1 has the potential to be the first disease-modifying treatment for early onset SCN2A GoF (gain-of-function) DEE.

### Example 2: Treatment of Early Onset SCN2A Developmental and Epileptic Encephalopathy Using Compound 1: A First-In-Patient Report in a Preterm Infant with Refractory Status Epilepticus

### Patient 1:

A preterm infant (29+4 weeks gestation; birthweight 1400 grams) was diagnosed prenatally using exome sequencing with a pathogenic SCN2A mutation. The pathogenic SCN2A variant identified was c.3986C>A p.(Ala1329Asp). The infant presented with status epilepticus (SE), as shown in FIG. 7. The infant had ongoing seizures since birth, confirmed by continuous EEG resembling SE, as well as a history of intrauterine seizures and arthrogryposis.

FIG. 7 shows representative aEEG traces demonstrating the subject's clinical course in the first weeks of life. Specifically, FIG. 7, Panel A shows representative aEEG traces in the subject showing a typical sawtooth pattern resembling EEG-status in week one. FIG. 7, Panel B shows representative aEEG traces in the subject demonstrating seizure reduction after several loading doses of phenytoin (arrow) in week three. FIG. 7, Panel C shows representative aEEG traces demonstrating that seizure reduction was not sustainable as status pattern reoccurred even when phenytoin levels were >40 μg/mL. Seizures were subclinical or motor seizures.

As shown in FIG. 7, anti-seizure treatment revealed only partial effect of high-dose sodium channel blockers (SCBs) and insufficient control of SE. Eligibility for Compound 1 treatment was evaluated using in silico protein structural modeling and in vitro electrophysiology studies aiming to ascertain GoF status and inform dosing strategies.

### Results

### Variant Characterization and GoF Confirmation

Voltage clamp experiments confirmed structural modeling predictions that the pathogenic p.Ala1329Asp variant interferes with binding of the inactivation motif that would lead to GoF via impaired inactivation and increased persistent current. Dynamic action potential clamp (DAPC) experiments, performed to assess the impact of the variant on intrinsic neuronal excitability, showed a large increase in action potential firing across the entire input range and significantly reduced rheobase compared to WT.

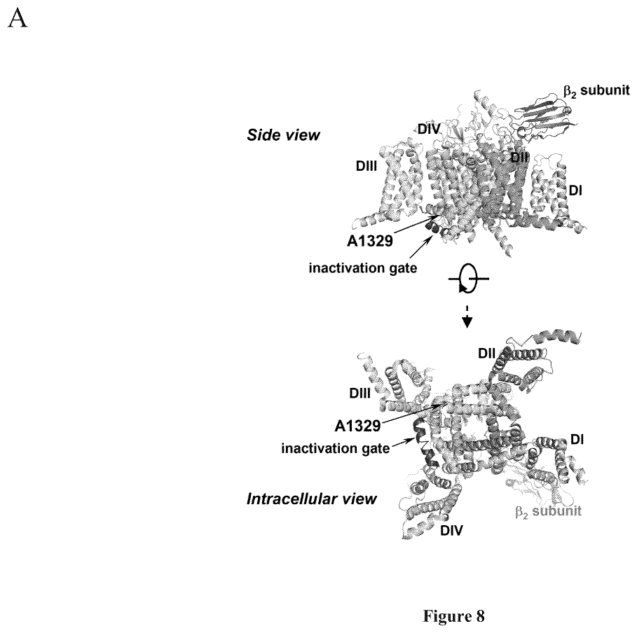

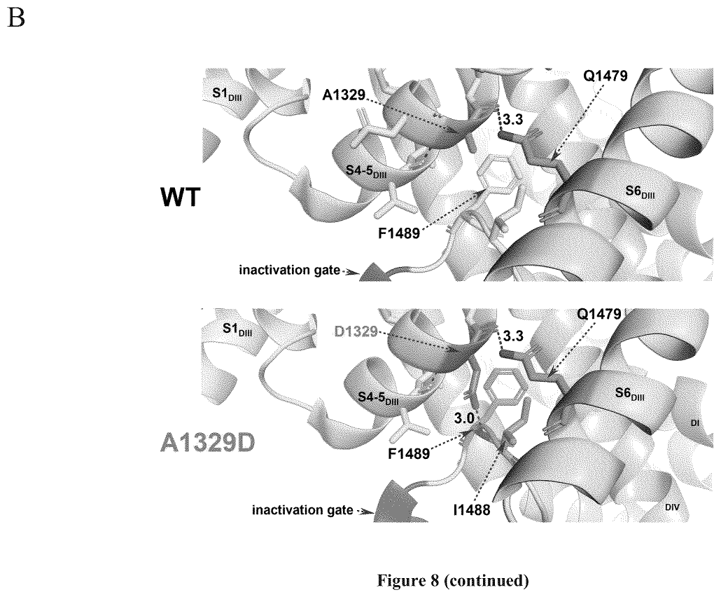

FIG. 8 shows the location of the A1329D Nav1.2 channel mutation. Specifically, FIG. 8, Panel A shows side and intracellular views of the 3D structure of Nav1.2 highlighting the A1329 residue in the intracellular linker between transmembrane segments S4 and S5 in domain III (S4-5_{DIII}). Also indicated are the four domains (DI-DIV), the inactivation gate and the β₂ subunit. FIG. 8, Panel B shows zoomed-in views of S4-5_{DIII} region, before and after in silico mutagenesis (top, WT; bottom, A1329D). The D1329-F1489 interaction is likely to affect the binding of the IFM inactivation motif to its receptor pocket, resulting in delayed inactivation and persistent current.

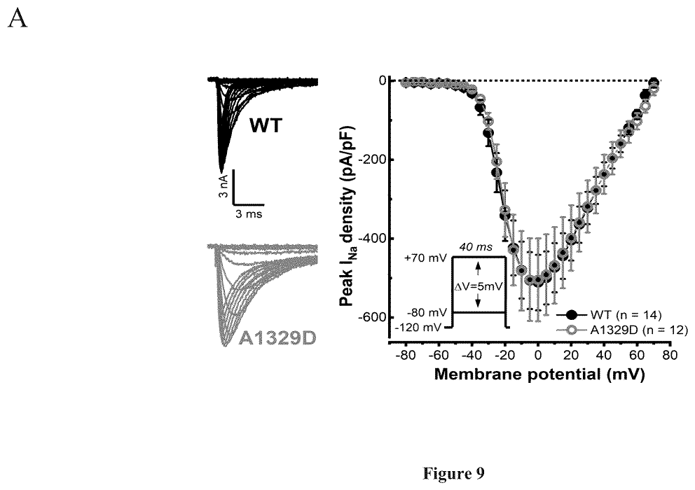

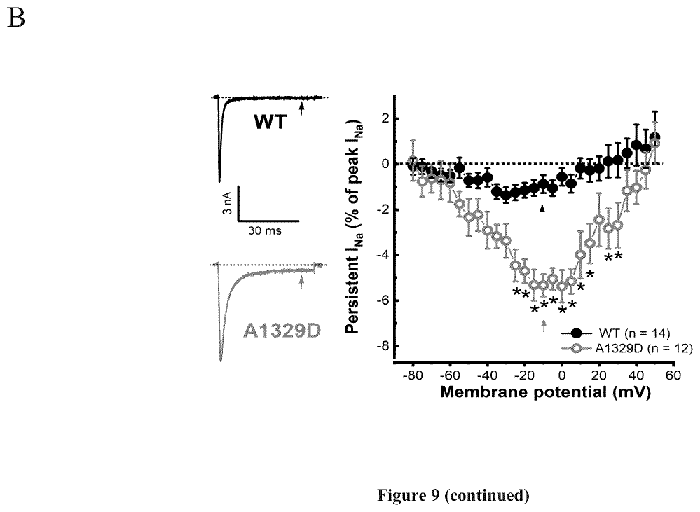

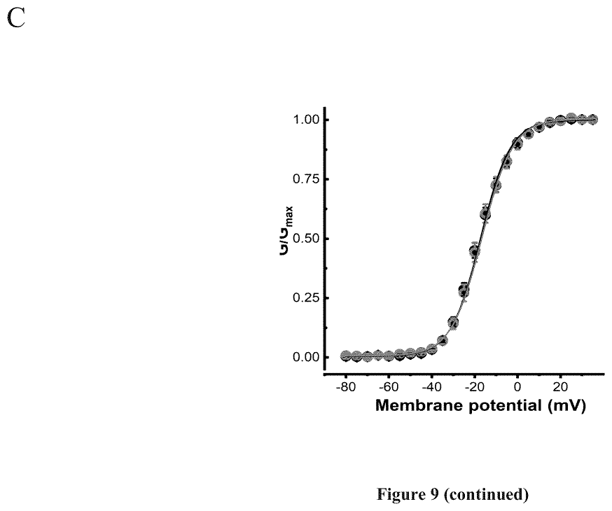

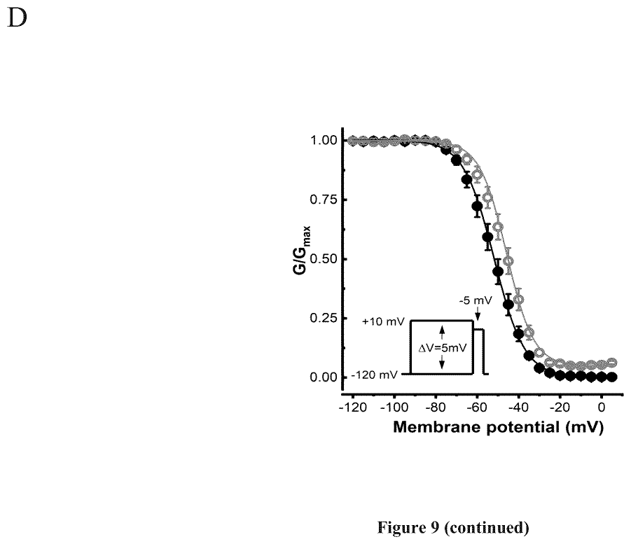

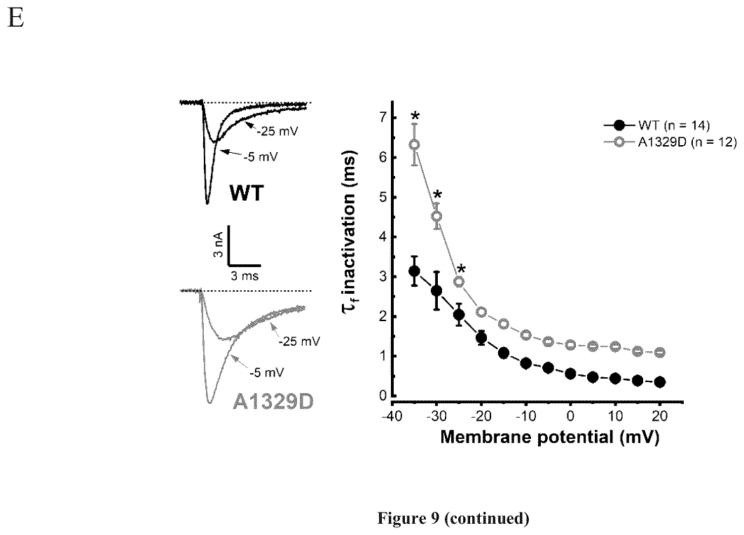

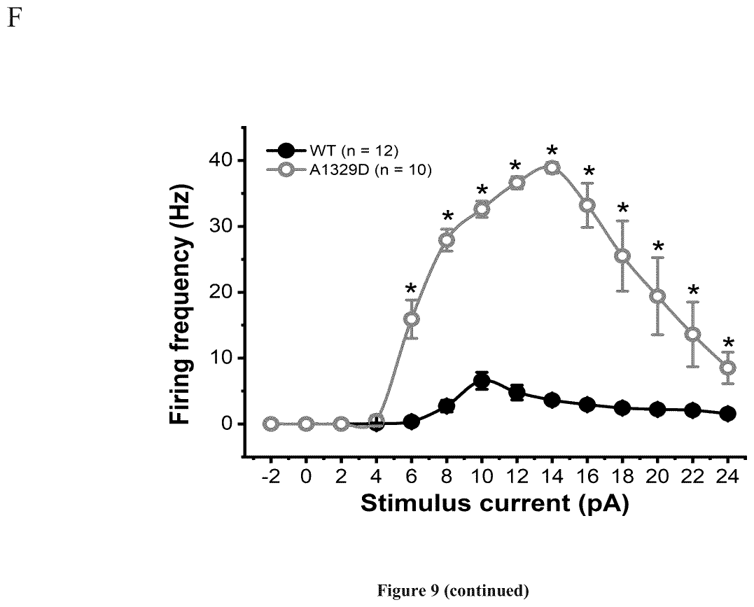

FIG. 9 shows biophysical characteristics of the A1329D variant and its impact on neuronal excitability relative to WT. In FIG. 9, data is presented as mean±SEM; *P<0.05 vs. WT. Specifically, FIG. 9, Panel A shows sodium current (I_{Na}) density-voltage relationships (inset voltage protocol). Representative I_{Na} traces are shown on the left. FIG. 9, Panel B shows persistent inward I_{Na}-voltage relationships. Representative I_{Na} traces elicited by −10 mV depolarizations are shown on the left. FIG. 9, Panel C shows voltage dependence of activation. FIG. 9, Panel D shows voltage dependence of inactivation. FIG. 9, Panel E shows dependence of the time course of I_{Na} inactivation on the membrane potential. Representative WT and A1329D I_{Na} traces elicited by −25 mV and −5 mV voltages are shown on the left. FIG. 9, Panel F shows input-output relationships for WT and A1329D variant.

Results Demonstrate Safety of Compound 1 and its Temporal Association with Seizure Reduction

Following confirmation of GoF status, treatment with Compound 1 was initiated when patient was 1 month and 2 weeks old. Administration of a total of 15 doses have been reported. The first 6 doses, administered over 17 weeks, were (1 mg, 0.5 mg, 1 mg during the first 5 weeks; followed by 4 mg, 8 mg and 8 mg at intervals of about 4 weeks. The total cumulative dose of Compound 1 administered over 23 weeks was 22.5 mg, and the total cumulative dose of Compound 1 administered over 28 weeks and 5 days was 30.5 mg. The total cumulative dose administered after 12 doses was 70.5 mg. The total cumulative dose administered after 15 doses was 94.5 mg of Compound 1. The dosing, including drug escalations and reductions, was determined by clinician. A summary of the dosing for Patient 1 is summarized below in Table 12:

TABLE 12
Dosing Regime for Patient 1

| | | | Dose Date | | | | |
|---|---|---|---|---|---|---|---|
| | Dose | | (days after | | Weight | Height | |
| # | Amount | | initial dose) | | (kg) | (cm) | Cumulative |
| 1 | 1 | mg | 0 | days | 2.485 | | |
| 2 | 0.5 | mg | 15 | days | 2.915 | | |
| 3 | 1 | mg | 31 | days | 3.350 | 51 | |
| 4 | 4 | mg | 80 | days | 4.380 | 52 | |
| 5 | 8 | mg | 122 | days | 5.280 | 58 | |
| | N/A* | | 131 | days* | 5.622 | 61 | |
| 6 | 8 | mg | 164 | days | 5.955 | 61.5 | |
| 7 | 8 | mg | 200 | days | 5.8 | 62 | |
| 8 | 8 | mg | 242 | days | 7 | 64 | |
| 9 | 8 | mg | 296 | days | 8 | 66 | |
| 10 | 8 | mg | 339 | days | 8.7 | 70 | |
| 11 | 8 | mg | 376 | days | 8.9 | 73 | |
| 12 | 8 | mg | 409 | days | 9.08 | 74 | |
| 13 | 8 | mg | 437 | days | 9.3 | 75 | 78.5 mg |
| 14 | 8 | mg | 466 | days | 9.6 | 76 | |
| 15 | 8 | mg | 495 | days | 9.8 | 76 | 94.5 mg |

*No dose on this date. Ht/Wt are for plasma sample collection

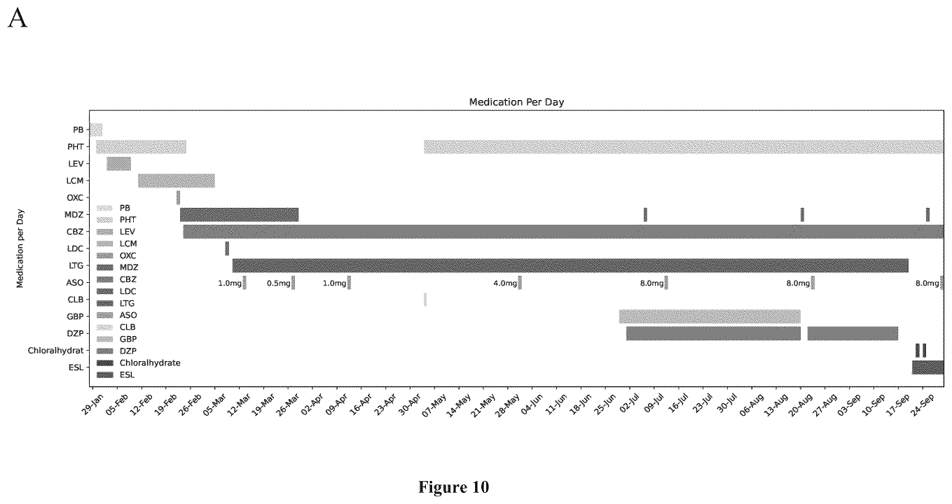

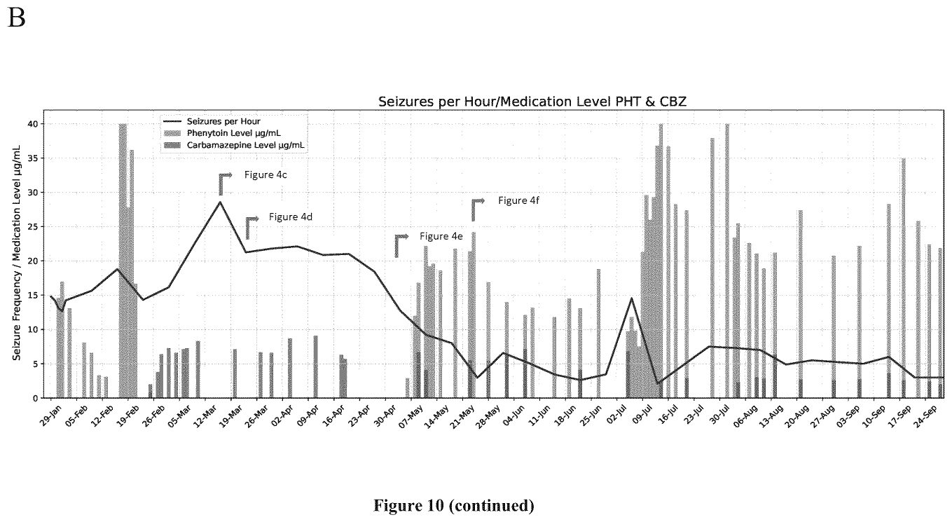

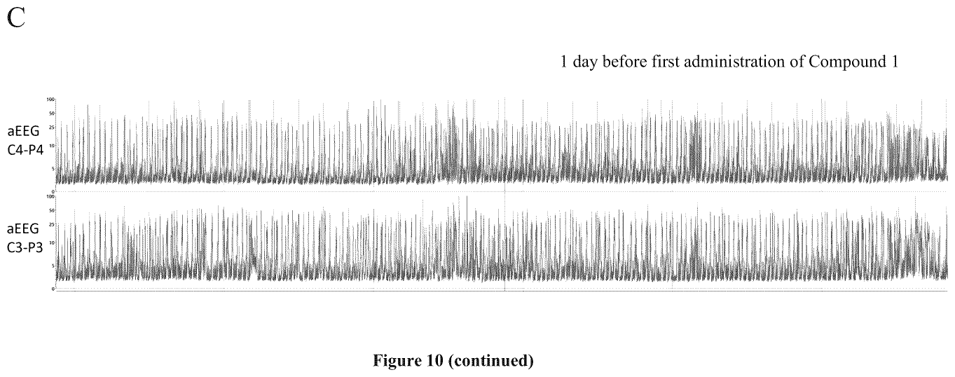

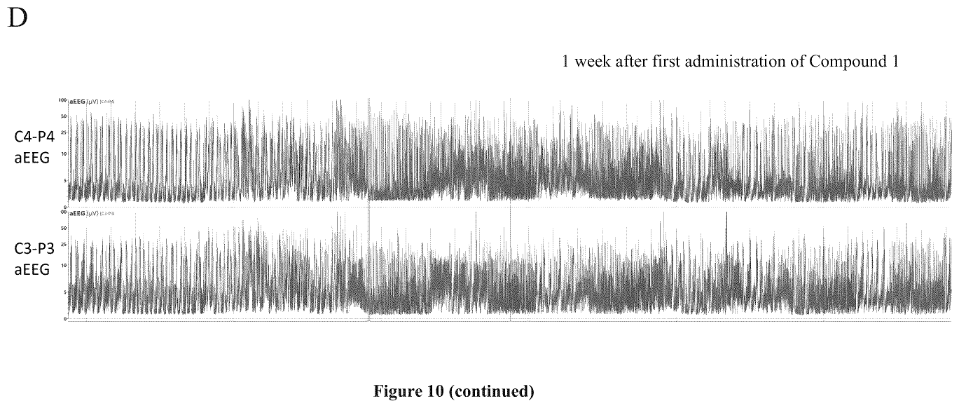

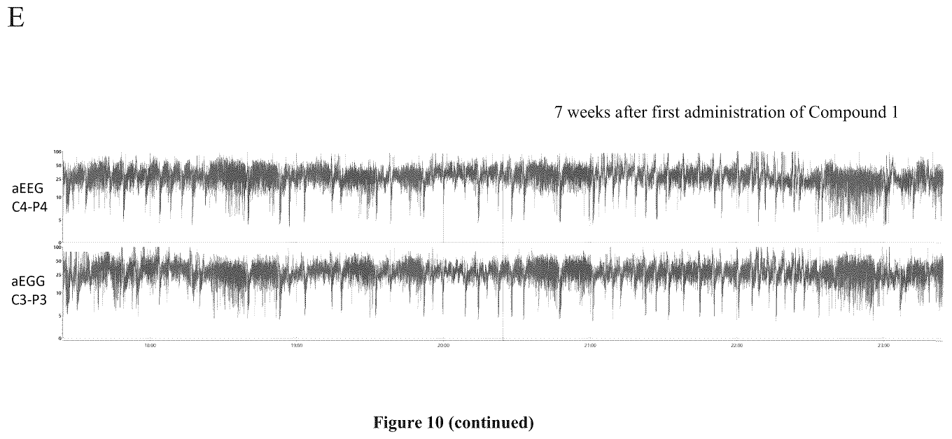

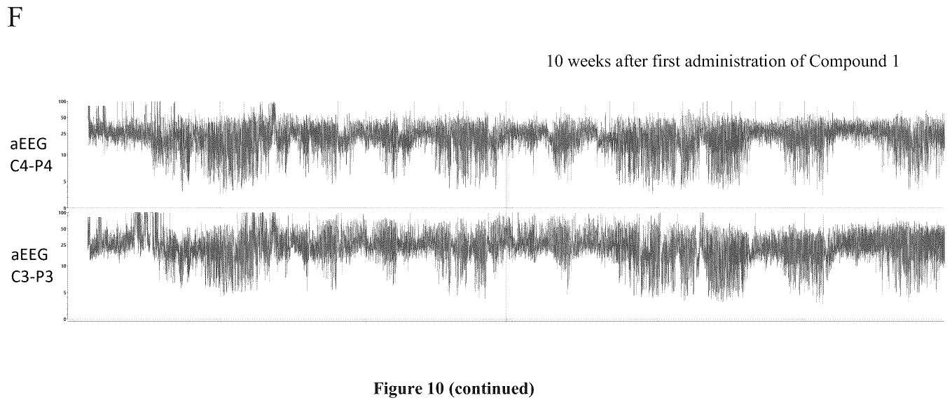

FIG. 10 shows patient clinical course following introduction of Compound 1 treatment regimen and effects on seizures. Specifically, FIG. 10, Panel A shows patient clinical course including high-dose SCBs and introduction of Compound 1 dosing regimen. Associated reduction in seizure frequency is shown (bottom). FIG. 10, Panel B is a bar graph showing the number of seizures per hour per medication level of phenytoin (PHT) and carbamazepine (CBZ). A total of 7 Compound 1 intrathecal doses were administered (30.5 mg total dose). FIG. 10, Panel C shows aEEG traces 1 day before the first administration of Compound 1 showing peak seizure frequency (status epilepticus). FIG. 10, Panel D shows aEEG traces 1 week after first administration of Compound 1. FIG. 10, Panel E shows aEEG traces 7 weeks after first administration of Compound 1. FIG. 10, Panel F shows aEEG traces 10 weeks after first administration of Compound 1.

The data shown in FIG. 10, Panels D-F demonstrate modulation and reduction of seizure activity (often in close timely relationship to SCB administration). It is also noted that seizure exacerbation between July 2 and 9 shown in the aEEG traces was due to urosepsis and concomitant decrease of SCB plasma levels.

A postnatal brain magnetic resonance imaging (MRI) at 5-weeks-old revealed T1 hyperintensities, periventricular thinning, prominent, extended lateral ventricles and periventricular medullary obliteration in addition to germinal matrix and periventricular leukomalacia possibly related to periventricular hemorrhagic infarction, demonstrating previous brain insults. A multitude of ASMs were administered immediately after birth and adjusted throughout the treatment period as deemed necessary by the treating physician, but the patient never adequately responded, sustaining refractory status epilepticus from birth until Compound 1 was administered.

Compound 1 treatment, in combination with best standard of care ASMs, was well tolerated with no severe or serious drug-related AEs up to a cumulative total of 94.5 mg over 16 months or 70 weeks.

To date, the patient has been administered a total of 15 doses of Compound 1 (1, 0.5, 1, 4, 8, 8, 8, 8, 8, 8, 8, 8, 8, 8, 8 mg) initiating at 6 weeks after birth, over a 70-week period. Dose levels were selected by the treating physician based on continuous evaluation of the infant's disease status while resident in the hospital. Compound 1 continues to be well tolerated, with no reported drug-related serious adverse events (SAEs) reported over the entire treatment period.

Importantly, no related AEs have been observed at the highest single doses of 8 mg (equivalent WT NHP dose of 0.8 mg with a total cumulative exposure of 3.05 mg). Following 6 doses with a cumulative exposure of 22.5 mg of dosing over 25 weeks, the CSF concentration of Compound 1 was determined to be 1.51 ng/mL. The exposures achieved demonstrate the safety and tolerability of Compound 1 in an infant, who had a bodyweight of 2 kg when the first dose was administered. Potential benefit of Compound 1 was evident soon after the first administration of Compound 1, where status epilepticus was interrupted intermittently 8 days after the first dose and ultimately ceased following continued dosing. Following the first administration of 4 mg of Compound 1, the patient stabilized, such that discharge from the intensive care unit for the first time was determined to be possible. A reduction in seizure frequency in excess of 50% was observed during follow-up assessments with seizure frequency and severity being markedly attenuated throughout the treatment period. Seizure frequence remains stable with ongoing dosing and was maintained after tapering phenytoin at age 14 months, with no worsening of neurodevelopment at age 18 months. Compound 1 was well-tolerated and no severe or serious adverse events were reported.

### Patient 2:

Patient 2 is an 8-year old patient who was diagnosed with SCN2A-related Gain-of-Function DEE. The patient had a history of refractory seizures, global cerebral atrophy, global developmental delay, frequent oculogyric movement, and severe dystonia while awake.

The patient has received a total of 10 doses over a period of about 9 months at approximately monthly (about 3-4 week in this case) intervals with the highest dose of 5 mg and cumulative exposure of 22 mg to date. There have been no drug-related AEs reported, and the procedure and dosing have been well tolerated. A summary of the dosing for Patient 2 is summarized below in Table 13:

TABLE 13
Dosing Regime for Patient 2

| | | Dose Date | | |
|---|---|---|---|---|
| | Dose | (days after | Weight | |
| # | Amount | initial dose) | (kg) | Cumulative |

| 1 | 1 | mg | 0 | days | | |
|---|---|---|---|---|---|---|
| 2 | 1 | mg | 22 | days | | |
| 3 | 1 | mg | 53 | days | | |
| 4 | 1 | mg | 83 | days | | |
| 5 | 1.5 | mg | 109 | days | 5.5 | mg |
| 6 | 1.5 | mg | 137 | days | | |
| 7 | 3 | mg | 166 | days | | |
| 8 | 3 | mg | 195 | days | | |
| 9 | 4 | mg | 223 | days | 17 | mg |
| 10 | 5 | mg | 250 | days | 22 | mg |

Prior to the administration of Compound 1, the patient experienced urinary calcinosis, likely secondary to ethosuximide, and previous attempts to wean the medication led to worsening of facial myoclonus and inability to feed. Two weeks after the second dose of Compound 1, a 0.5 mL weekly reduction of ethosuximide was achieved and facial myoclonus subsided during waking hours.

Also, after the second dose, nystagmus was reportedly much less, tone was much improved, and administration of Botulinim toxin was ceased. Additionally, after administration of Dose 2, the patient was reported to have a drastic decrease in seizures and previous full tonic-clonic seizures appeared as subtle tongue movement with the jaw (reportedly how the tonic-clonic seizures previously began), but the tongue movement did not progress to tonic-clonic seizure. It was further reported by the care team that after Dose 2, the patient appeared happy and calm as compared to prior to treatment with Compound 1 when the patient was more agitated and exhibited more crying and screaming. After Dose 2, the patient was observed to make subtle hand movements not made before, and it was reported that she moved her arm to greet a visitor. The patient was also observed by teachers at school as being more alert than prior to treatment. Daily seizure counts are not currently being provided; however, a 24-hour EEG, conducted between Dose 2 and Dose 3, showed significantly fewer clinical and electrographic seizures; multifocal and generalized epileptic discharges remained abnormal.

In general, Compound 1 has been well tolerated over the administrations. Additionally, there have been several notable improvements in quality-of-life, as communicated by caregivers and medical personnel who provide support to the patient, including that:
the family reported a reduction in tonic-clonic seizure intensity as well as improvement in attention and vocalization. The family reported that the patient is smiling more often;
the family reported that the patient is more comfortable in a wheelchair and is more tolerant of sensory stimuli in general. As a result, they have begun going to restaurants;
an occupational therapist reported improved range of motion and botulinic toxin administration was withheld;
a speech therapist was able to provide liquids without thickening and reported that gazing communication and alertness improved;
   Digestion has improved significantly, and suppositories are able to be withheld.

### Patient 3:

Patient 3 is a patient from Example 1 (ID No. 2001) and was granted continued access to Compound 1. A summary of the continued dosing for Patient 3 is summarized below in Table 14:

TABLE 14
Dosing Regime for Patient 3

| | | Dose Date | | | |
|---|---|---|---|---|---|
| | Dose | (days after | | Weight | |
| # | Amount | initial dose) | | (kg) | Cumulative |
| 1 | 1 mg | 0 | days | 20 | |
| 2 | 1 mg | 35 | days | 20 | |
| 3 | 1 mg | 63 | days | 20 | 3 mg |
| 4 | 1 mg | 98 | days | 20 | |
| 5 | 1 mg | 132 | days | | 5 mg |

Compound 1 continues to be well tolerated by Patient 3.

### Patient 4:

Patient 4 is a patient from Example 1 (ID No. 2004) and was granted continued access to Compound 1. A summary of the continued dosing for Patient 4 is summarized below in Table 15:

TABLE 15
Dosing Regime for Patient 4

| | | Dose Date | | | |
|---|---|---|---|---|---|
| | Dose | (days after | | Weight | |
| # | Amount | initial dose) | | (kg) | Cumulative |
| 1 | 1 mg | 0 | days | 19 | |
| 2 | 1 mg | 31 | days | 19 | |
| 3 | 1 mg | 79 | days | 19 | 3 mg |
| 4 | 1 mg | 114 | days | | 4 mg |
| 5 | 1 mg | 145 | days | | |

Compound 1 continues to be well tolerated by Patient 4.

### CONCLUSION

First-in-patient findings demonstrate Compound 1 to be the first disease-modifying treatment for early onset GoF SCN2A-DEE, with early clinical experience in combination with SCBs indicating safety and a temporal association with seizure reduction, including cessation of previous refractory SE.

What is claimed is:

1. A method of reducing frequency of seizures experienced by a subject with early onset SCN2A Developmental and Epileptic Encephalopathy (DEE), said method comprising administering to said subject an effective amount of an oligomeric compound, wherein:
the SCN2A DEE is caused by a gain-of-function mutation in SCN2A gene;
the oligomeric compound comprises a 6-10-4 MOE gapmer having a sequence (from 5′ to 3′) of CCACGACATATTTTTCTACA (SEQ ID NO: 3);
wherein each of nucleosides 1-6 and 17-20 (from 5′ to 3′) are 2′-MOE nucleosides and each of nucleosides 7-16 are 2′-β-D-deoxynucleosides;
wherein the internucleoside linkages between nucleosides 2 to 3, 3 to 4, 4 to 5, 5 to 6, 6 to 7, and 17 to 18 are phosphodiester internucleoside linkages, the internucleoside linkages between nucleosides 1 to 2, 7 to 8, 8 to 9, 9 to 10, 10 to 11, 11 to 12, 12 to 13, 13 to 14, 14 to 15, 15 to 16, 16 to 17, 18 to 19, and 19 to 20 are phosphorothioate internucleoside linkages; and
each cytosine is a 5-methyl cytosine; and
wherein the oligomeric compound is administered at a dose of about 0.1 mg to about 20 mg.

2. The method of claim 1, wherein the oligomeric compound is administered at a dose of about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 2.5 mg, about 3 mg, about 3.5 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg or about 20 mg.

3. The method of claim 1, wherein the oligomeric compound is administered at a dose of about 0.5 mg, about 1 mg, about 4 mg, about 8 mg, about 12 mg or about 15 mg.

4. The method of claim 3, wherein the oligomeric compound is administered at a dose of about 1 mg.

5. The method of claim 1, wherein the oligomeric compound is administered about once per month.

6. The method of claim 1, wherein the seizures are selected from the group consisting of focal motor seizures, tonic seizures, generalized tonic-clonic seizures and myoclonic seizures.

7. The method of claim 1, wherein administration of the oligomeric compound results in a decrease in the average number of daily seizures experienced by the subject in a 28-day period, as compared to the average number of daily seizures experienced by the subject prior to administration of the oligomeric compound.

8. The method of claim 1, wherein administration of the oligomeric compound results in a reduction in the number of seizures experienced by the subject in a 28-day period by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, as compared to the number of seizures experienced by the subject prior to administration of the oligomeric compound.

9. The method of claim 1, wherein the subject is a human.

10. The method of claim 9, wherein the subject is between 2 and 18 years old.

11. The method of claim 9, wherein the subject is between 0 and 24 months old.

12. The method of claim 11, wherein the subject is a newborn.

13. The method of claim 12, wherein the newborn is a premature newborn.

14. The method of claim 1, wherein the gain-of-function mutation in SCN2A gene is selected from the group consisting of L210Q, A263V, E430A, R1882Q, G879R, Q1479H, V423L, G1593R, K1502N, V1601L, G211D, S1780I, D343H and A1329D.

15. The method of claim 14, wherein the gain-of-function mutation is A1329D.

16. The method of claim 1, wherein the oligomeric compound comprises a modified oligonucleotide represented by the following chemical notation: ^{m}Cₑₛ^{m}CₑₒAₑₒ^{m}CₑₒGₑₒAₑₒ^{m}C_{ds}A_{ds}T_{ds}A_{ds}T_{ds}T_{ds}T_{ds}T_{ds}T_{ds}^{m}C_{ds}TₑₒAₑₛ^{m}CₑₛAₑ (SEQ ID NO: 3), wherein:
A=an adenine nucleobase,
^{m}C=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

17. The method of claim 16, wherein the oligomeric compound is a modified oligonucleotide represented by the following chemical structure:

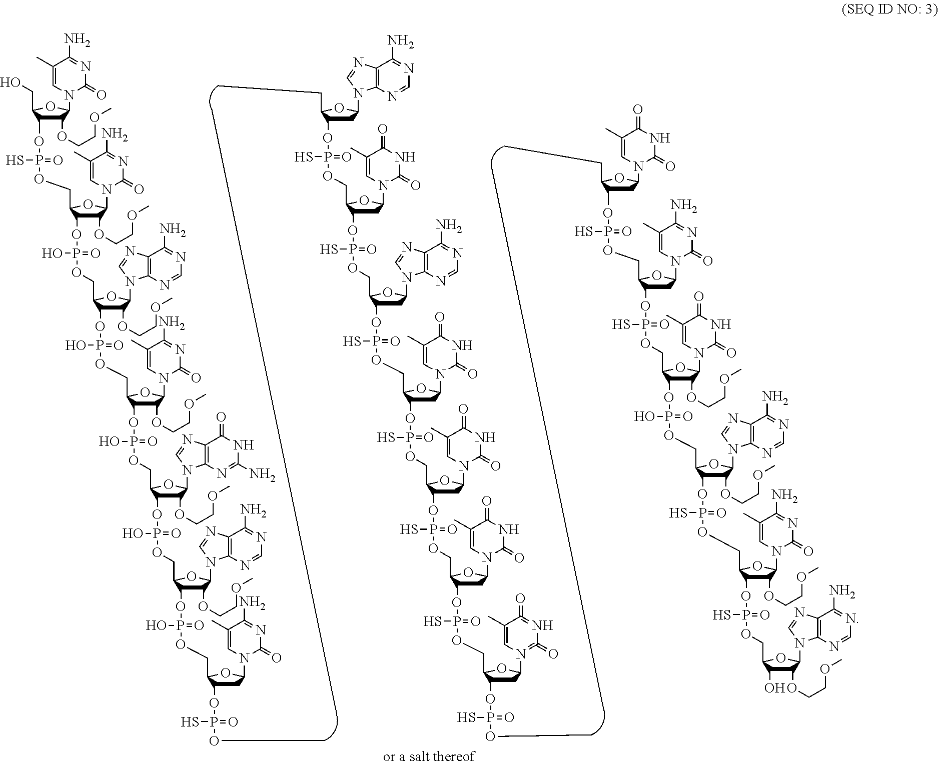

18. The method of claim 17, wherein the modified oligonucleotide is a sodium salt or a potassium salt.

19. The method of claim 18, wherein the modified oligonucleotide is a sodium salt.

20. The method of claim 19, wherein the modified oligonucleotide represented by the following chemical structure:

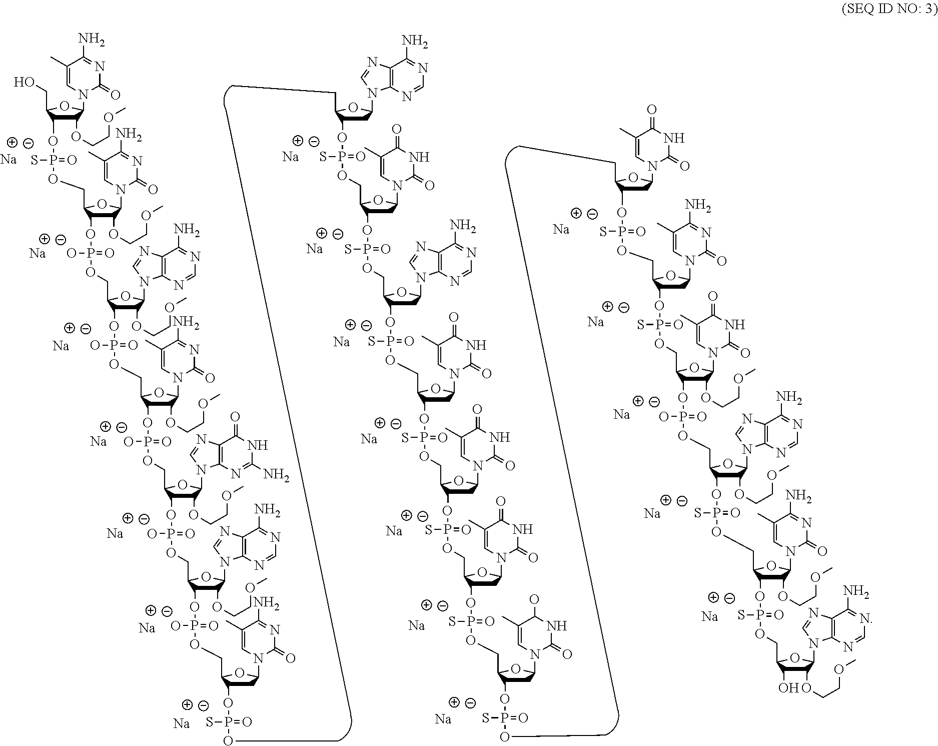

21. The method of claim 1, wherein the oligomeric compound is administered as a part of a pharmaceutical composition comprising the oligomeric compound and a pharmaceutically acceptable diluent or carrier.

22. The method of claim 21, wherein the pharmaceutically acceptable diluent is artificial CSF (aCSF) or phosphate-buffered saline (PBS).

23. The method of claim 22, wherein the pharmaceutically acceptable diluent is aCSF.

24. The method of claim 1, wherein the oligomeric compound is administered intrathecally.
